(19) United States
(12) Patent Application Publication
(10) Pub. No.: US 2026/0184754 A1
(43) Pub. Date: Jul. 2, 2026

(54) POLYNUCLEOTIDE ENCODING MEMBRANE-TYPE CYTOKINE AND INTRACELLULAR DOMAIN OF TNF RECEPTOR SUPERFAMILY MOLECULE

(71) Applicant: Daiichi Sankyo Company, Limited, Chuo-ku, Tokyo (JP)

(72) Inventors: Kazunori YOSHIKIYO, Chuo-ku, Tokyo (JP); Koji SUDA, Chuo-ku, Tokyo (JP); Hiroshi KARASAWA, Chuo-ku, Tokyo (JP); Sayaka YOSHIDA, Chuo-ku, Tokyo (JP); Hiroaki TAGAYA, Chuo-ku, Tokyo (JP)

(73) Assignee: Daiichi Sankyo Company, Limited, Chuo-ku, Tokyo (JP)

(21) Appl. No.: 18/855,298

(22) PCT Filed: Apr. 13, 2023
(86) PCT No.: PCT/JP2023/014944

§ 371 Date: Oct. 8, 2024

(30) Foreign Application Priority Data
Apr. 14, 2022 (JP) 2022-067114

(51) Int. Cl. C07K 14/54; A61K 35/17; A61K 40/11; A61K 40/15; A61K 40/31; C07K 14/55; C07K 14/705; C12N 5/0783; C12N 5/10; C12N 15/11; C12N 15/85

(52) U.S. Cl. CPC C07K 14/5443; A61K 35/17; A61K 40/11; A61K 40/15; A61K 40/31; C07K 14/5418; C07K 14/55; C07K 14/70517; C07K 14/70521; C07K 14/70578; C12N 5/0636; C12N 5/0646; C12N 5/10; C12N 15/11; C12N 15/85; C07K 2319/03; C12N 2510/00; C12N 2830/34

(57) ABSTRACT

An object of the present invention is to provide an immune cell capable of favorably surviving and/or proliferating under a cytokine-free culture condition, or in the living body in which a high concentration of cytokine cannot be expected, and a molecule or the like for preparing the immune cell. Membrane-bound IL2, membrane-bound IL7, or membrane-bound IL15, and a TNFRSF molecule-derived intracellular domain are expressed in the immune cell as a combination of different polypeptides, or are both expressed in an immune cell as the single polypeptide.

### TECHNICAL FIELD

The present invention relates to a polynucleotide capable of expressing, in an immune cell, IL2, IL7, or IL15 tethered to a cell membrane (hereinafter, sometimes referred to as “membrane-bound IL2 or IL2TM”, “membrane-bound IL7 or IL7TM”, and “membrane-bound IL15 or IL15TM”, respectively), and a TNF receptor superfamily (TNFRSF) molecule-derived intracellular domain as a combination of different polypeptides, or as a single polypeptide containing the both; an expression vector containing the polynucleotide; an immune cell into which the expression vector has been introduced; a medicament containing the immune cell; the polypeptide or the combination of the polypeptides; an immune cell in which the single polypeptide or the combination of the polypeptides are expressed on the cell membrane; a method for preparing a cell population containing the immune cell using the expression vector; and the like.

### BACKGROUND ART

Immunity is classified into innate immunity and acquired immunity. The innate immunity is a rapid immune response against a wide range of pathogens mainly of immune cells such as a natural killer cell (NK cell), a macrophage, and a granulocyte. On the other hand, the acquired immunity is a selective and effective immune response mainly of immune cells such as a dendritic cell, a B cell, and a T cell.

In the field of regenerative medicine, adoptive immune cell therapy (also referred to as adoptive immunotherapy) in which an immune cell present in the body of a patient is collected, proliferated outside the body, and returned to the body again has been attempted to be developed. In the adoptive immune cell therapy, a novel function can be imparted to an immune cell by employing genetic modification technology. For example, attempts have been made for developing a T cell receptor (TCR) recognizing a major histocompatibility antigen complex (MHC) presenting an intracellular antigen specific to a cancer cell, and an immune cell in which high affinity CD16 is expressed. Besides, attempts have been made for expressing, in an immune cell, a chimeric polypeptide derived from a plurality of different proteins. As such a chimeric polypeptide, for example, a chimeric antigen receptor (CAR) in which an antibody recognizing a cancer antigen and an activation region of a T cell are linked to each other, and a chimeric autoantibody receptor (CAAR) in which an antigen being recognized by an autoantibody and an activation region of a T cell are linked to each other are known (Non Patent Literature 1). In recent years, attentions are being paid to a T cell expressing a CAR (CAR-T cell) because of a very high medicinal effect thereof against blood cancer.

The T cell is activated when a signal caused by recognition of an antigen via a TCR/CD3 complex, and a signal caused by an antigen-non-specific co-stimulatory molecule (also referred to as a co-stimulatory factor, or a co-stimulatory molecule) are both transmitted. In a tumor cell, however, expression of a co-stimulatory molecule is known to reduce, and therefore, it has been reported that even when a CAR-T cell can recognize a target tumor cell, sufficient signal from a co-stimulatory molecule is not transmitted, and hence the CAR-T cell cannot be sufficiently activated, and an antitumor effect cannot be obtained. Therefore, in these days, a gene of a co-stimulatory molecule is incorporated into a CAR gene, so as to develop a CAR-T cell that transmits a co-stimulatory signal through antigen recognition via a CAR. The type, the number, the combination and the like of genes of co-stimulatory molecules to be incorporated into the CAR gene have been changed, so as to improve the survival rate and proliferation ability of the CAR-T cell, and to impart and enhance various functions such as differentiation induction to a memory T cell (Non Patent Literature 2).

On the other hand, it has been also reported that the CAR-T cell therapy does not exhibit a sufficient medicinal effect on solid cancer. One reason is presumed as follows: Due to a difference in localization between a transplanted CAR-T cell and a target tumor cell, the frequency of contact between the CAR-T cell and an antigen is lower in solid cancer than in blood cancer, and hence a proliferation signal is not caused. As a result, a necessary amount of cytokine is not supplied (Non Patent Literatures 3 and 4). Therefore, there is a demand for development of a CAR-T cell that can survive, proliferate, and be activated even under an environment with low frequency of contact with an antigen, and for development of adoptive immune cell therapy for cancer using the CAR-T cell (namely, cancer immunotherapy).

A cytokine is widely known to play a significant role in differentiation, maturation, activation, and proliferation of various immune cells. For example, as a cytokine involved in survival and proliferation of an NK cell and a T cell, the common γ chain (γc) cytokine family is known. The γc cytokine family binds to receptors containing a common γc subunit, and is composed of interleukin 2 (IL2), interleukin 4 (IL4), interleukin 7 (IL7), interleukin 9 (IL9), interleukin 15 (IL15), and interleukin 21 (IL21). The receptor of the γc cytokine family is composed of 2 or 3 components commonly using γc, and expresses in an immune cell on which the cytokine acts. In the adoptive immune cell therapy, technologies for expressing, in an immune cell, these cytokines, and chimeric polypeptides of these cytokines and cytokine receptors are known.

The co-stimulatory molecule is roughly divided into an immunoglobulin superfamily (IgSF) molecule, and a TNF/TNF (tumor necrosis factor) receptor superfamily (TNFRSF) molecule. As the IgSF molecule, B7/CD28 family molecules (CD28, CTLA4, PD1, and ICOS), the TIM family (TIM1, and TIM3), and the CD2/SLAM family (SLAM, CD2, CD84, CRACC, and BLAME) and the like are known. As the TNFRSF molecule, CD137 (4-1BB), CD134 (OX40), HVEM, CD27, TNFR2, CD30, DR3, GITR, LTβR, and the like are known.

Non Patent Literature 5 describes a T cell enhanced in survival and proliferation obtained by gene introduction of an expression vector of a chimeric polypeptide containing IL15 linked to IL15Rα (hereinafter, sometimes referred to as “IL15-IL15Rα”) to express the chimeric polypeptide therein. This document does not, however, specifically describe co-expression of a cytokine tethered to a cell membrane (hereinafter sometimes referred to as “membrane-bound cytokine”) and a TNFRSF molecule. In this document, IL15-IL15Rα and CD19 CAR (CAR obtained by using CD28 as a co-stimulatory molecule, and CD3ζ as an ITAM (Immunoreceptor Tyrosine-based Activation Motif) intracellular signaling region) are co-expressed.

Patent Literature 6 describes a T cell enhanced in survival and proliferation obtained by gene introduction of an expression vector of membrane-bound IL7 to express the membrane-bound IL7. This document does not, however, specifically describe co-expression of a membrane-bound cytokine and a TNFRSF molecule. In this document, the membrane-bound IL7 and CD19 CAR are co-expressed, but specific structures of a co-stimulatory molecule and an ITAM intracellular signaling region used by the CAR are not described.

Patent Literature 1 describes an NK cell enhanced in survival and proliferation obtained by gene introduction of an expression vector of membrane-bound IL15 to express the membrane-bound IL15. Co-expression of a membrane cytokine and a TNFRSF molecule is, however, not specifically described.

Patent Literature 2 describes a T cell enhanced in survival and proliferation obtained by expressing IL15 or IL15-IL15Rα bound by an inactivation domain. Co-expression of a membrane-bound cytokine and a TNFRSF molecule is, however, not specifically described.

Patent Literature 3 describes a T cell obtained by gene introduction of TeIL21/15. It is also described that such a T cell is enhanced in survival and proliferation, and is improved in antitumor activity. Co-expression of a membrane-bound cytokine and a TNFRSF molecule is, however, not specifically described.

Patent Literature 4 describes an NK cell obtained by co-expressing, in a signaling domain of NKG2D CAR (CAR using CD3 as an ITAM intracellular signaling region), a cytotoxic signal transduction complex containing OX40, 4-1BB, or CD28 domain, and membrane-bound IL15. Co-expression of a co-stimulatory molecule and a membrane-bound cytokine separately from the cytotoxic signal transduction complex is, however, not specifically described.

Patent Literature 5 describes an NK cell and a T cell obtained by co-expressing, in a signaling domain, a CAR containing a co-stimulatory molecule-derived intracellular domain such as OX40 and a CD3ζ intracellular domain, and membrane-bound IL15. Co-expression of a TNFRSF molecule and a membrane-bound cytokine separately from the CAR is, however, not specifically described.

### CITATION LIST

### Patent Literature

Patent Literature 1: International Publication No. WO2015/174928
Patent Literature 2: International Publication No. WO2018/161026
Patent Literature 3: International Publication No. WO2019/157130
Patent Literature 4: International Publication No. WO2020/056045
Patent Literature 5: International Publication No. WO2020/247392

### Non Patent Literature

Non Patent Literature 1: Ellebrecht et al, Science 2016; 353 (6295): 179-84
Non Patent Literature 2: Kershaw et al, Nat Rev Cancer. 2013 August; 13(8):525-41.
Non Patent Literature 3: Knochelmann et al, Front Immunol. 2018 Jul. 27; 9:1740.
Non Patent Literature 4: Junghans et al, Cancer Gene Ther. 2017 March; 24(3):89-99.
Non Patent Literature 5: Hurton et al, PNAS. Nov. 14, 2016
Non Patent Literature 6: Hurton et al, Blood (2009) 114 (22):3035.

### SUMMARY OF INVENTION

### Technical Problem

Adoptive immune cell therapy is autologous cell therapy using a patient-derived immune cell in many cases, and is desired to exhibit a therapeutic effect within the body of any patient. Therefore, the immune cell used for the treatment is demanded to be able to proliferate without influence from the patient. Besides, since a high concentration of cytokine as that used in ex vivo culture cannot be expected in the living body, in order to exhibit a sufficient therapeutic effect, the immune cell is required to at least survive, and preferably proliferate even under a condition of a low concentration cytokine.

As described in Example 1 below, however, when membrane-bound IL15 or “IL15-IL15Rα” was expressed in T cells derived from peripheral blood mononuclear cells (PBMC) of a plurality of donors, and the resultant was cultured in a cytokine-free medium (also referred to as the “culture fluid”), it was found that the cell proliferation ability of the T cell largely varied among donors, and that the cell number of T cells was reduced at an initial stage of the culture in some donors. Besides, in most of the donors, the cell proliferation ability of the T cell was reduced 2 weeks or more after the culture, and it was thus found that the cell proliferation ability of the T cell cannot be retained for a long period of time. In other words, it was found that a sufficient cell survival and proliferation effect cannot be always obtained in the absence of a cytokine merely by expressing membrane-bound IL15 or “IL15-IL15Rα” in a T cell. Therefore, even when such a T cell is used in the adoptive immune cell therapy, a sufficient effect cannot be probably expected for a large number of patients, and hence, novel technology for further enhancing the function (for example, cell proliferation ability) of an immune cell to be used in treatment, and further increasing the effect of the adoptive immune cell therapy was required. Besides, improvement of the function of an immune cell, such as proliferation and survival for a long period of time, was not expected because a bond to a target antigen is necessary for signal transduction of a co-stimulatory molecule in a CAR-T cell having a membrane-bound cytokine expressed therein, and a signal from an ITAM intracellular signaling region such as CD3ζ is transmitted to the T cell simultaneously with a signal of the co-stimulatory molecule, and hence the immune cell expressing the CAR itself exhausts or is damaged.

An object of the present invention is to provide an immune cell capable of favorably surviving and/or proliferating under a cytokine-free culture condition, or in the living body in which a high concentration of cytokine cannot be expected, and a molecule or the like for preparing the immune cell.

### Solution to Problem

The present inventors have been continuously earnestly making studies to solve the above-described problems. In the process, the following has been found: When membrane-bound IL2, membrane-bound IL7, or membrane-bound IL15, and a TNFRSF molecule-derived intracellular domain are expressed in an immune cell as a combination of respective different polypeptides (namely, as a present polypeptide combination), or both expressed in an immune cell as the single polypeptide (namely, a present membrane-bound cytokine-TNFRSF molecule chimeric ligand), the cell proliferation under a cytokine-free culture condition is improved as compared with an immune cell expressing only membrane-bound IL2, membrane-bound IL7, or membrane-bound IL15; an immune cell expressing membrane-bound IL15 and a CD28 family molecule-derived intracellular domain (a co-stimulatory molecule except for the TNFRSF molecule) as the single polypeptide; or an immune cell expressing a membrane-bound cytokine except for membrane-bound IL2, membrane-bound IL7, and membrane-bound IL15, and a TNFRSF molecule-derived intracellular domain as the single polypeptide.

It has been also confirmed that when a ligand in which a ligand molecule tethered to a cell membrane (hereinafter, sometimes referred to as a “membrane-bound ligand molecule”) is linked to a TNFRSF molecule-derived intracellular domain (hereinafter, sometimes referred to as a “present membrane-bound ligand molecule-TNFRSF molecule chimeric ligand”), and membrane-bound IL15 are co-expressed in an immune cell, the cell proliferation is improved under a cytokine-free culture condition as compared with an immune cell co-expressing a TNFRSF molecule-derived intracellular domain not containing a membrane-bound ligand molecule, and membrane-bound IL15.

Furthermore, it has been confirmed, in an in vivo system, that an immune cell expressing a present membrane-bound cytokine-TNFRSF molecule chimeric ligand can favorably survive and/or proliferate in the living body no matter whether it has been pre-cultured for several days before administration to the living body, and without depending on a donor from which the immune cell is derived.

It has been also confirmed, in in vitro and in vivo systems, that a co-stimulatory molecule-free CAR-T cell (first generation CAR-T cell) expressing a present membrane-bound cytokine-TNFRSF molecule chimeric ligand has strong and persistent cytotoxic activity against cancer as compared with an existing second generation CAR-T cell.

It has been also confirmed that when a cell population containing an immune cell expressing a present membrane-bound cytokine-TNFRSF molecule chimeric ligand is cultured in the absence of IL15, IL2, and IL7, a cell population containing the immune cell at a high purity can be prepared.

The present invention has been accomplished based on these finding.

Specifically, the present invention provides the following:
[1] A polynucleotide comprising:
an extracellular domain encoding region encoding an extracellular domain containing an amino acid sequence derived from a ligand protein (which ligand protein hereinafter sometimes referred to as the “present cytokine”, typical example being IL15, IL2, or IL7) that binds to a receptor of IL15, IL2, or IL7, wherein the binding of the ligand protein to the receptor on an immune cell transmits a signal similar to a binding signal of the cytokine into the immune cell via the receptor (which extracellular domain will be hereinafter sometimes referred to as a “present cytokine extracellular domain”) (which extracellular domain encoding region will be hereinafter sometimes referred to as a “present cytokine extracellular domain encoding region”); and
an intracellular domain encoding region encoding an intracellular domain containing a region derived from an intracellular domain of a TNF receptor superfamily (TNFRSF) molecule, but not containing an ITAM (Immunoreceptor Tyrosine-based Activation Motif) intracellular signaling region (which intracellular domain will be hereinafter sometimes referred to as a “present TNFRSF molecule intracellular domain”) (which intracellular domain encoding region will be hereinafter sometimes referred to as a “present TNFRSF molecule intracellular domain encoding region”),
wherein (a) the polynucleotide is designed as an extracellular domain-intracellular domain-containing molecule gene (which gene will be hereinafter sometimes referred to as a “present membrane-bound cytokine-TNFRSF molecule chimeric ligand encoding region”) that enables to express, on the immune cell, a polypeptide containing both of the extracellular domain and the intracellular domain (which polypeptide will be hereinafter sometimes referred to as a “present membrane-bound cytokine-TNFRSF molecule chimeric ligand”) when the extracellular domain and the intracellular domain are linked to each other via a transmembrane domain, or
(b) the polynucleotide is designed as an extracellular domain-containing molecule gene and an intracellular domain-containing molecule gene (which genes will be hereinafter sometimes referred to as the present polypeptide combination encoding regions) that enable to express, on the immune cell, a combination of a first polypeptide containing the extracellular domain and a second polypeptide containing the intracellular domain (which polypeptide combination will be hereinafter sometimes referred to as a “present polypeptide combination”) when the extracellular domain and the intracellular domain are linked to a first cell membrane binding domain and a second cell membrane binding domain, respectively (which the polynucleotide will be hereinafter sometimes referred to as a “present polynucleotide”).
[2] The polynucleotide according to [1], wherein the IL15, IL2, or IL7, and the TNFRSF molecule are derived from a human, a mouse, or a rat.
[3] The polynucleotide according to [1] or [2], wherein the extracellular domain containing an amino acid sequence derived from the ligand protein that binds to an IL15 receptor contains an amino acid sequence having at least 80% sequence identity with an amino acid sequence shown in SEQ ID NO: 22, and retains activity of binding to the IL15 receptor; the extracellular domain containing an amino acid sequence derived from the ligand protein that binds to an IL2 receptor contains an amino acid sequence having at least 80% sequence identity with an amino acid sequence shown in SEQ ID NO: 17, and retains activity of binding to the IL2 receptor; and the extracellular domain containing an amino acid sequence derived from the ligand protein that binds to an IL7 receptor contains an amino acid sequence having at least 80% sequence identity with an amino acid sequence shown in SEQ ID NO: 20, and retains activity of binding to the IL7 receptor.
[4] The polynucleotide according to any one of [1] to [3], wherein the TNFRSF molecule is TNFR2, OX40, HVEM, CD27, or CD137.
[5] The polynucleotide according to any one of [1] to [4], wherein the immune cell is a T cell or a natural killer cell.
[6] The polypeptide according to any one of [1] to [5], wherein the transmembrane domain is a CD8α-derived transmembrane domain, or a CD28-derived transmembrane domain.
[7] The polynucleotide according to any one of [1] to [6], wherein the extracellular domain and the transmembrane domain are linked to each other via a linker peptide.
[8] The polynucleotide according to [7], wherein the linker peptide is a CD8α-derived linker peptide, or a CD28-derived linker peptide.
[9] The polynucleotide according to any one of [1] to [5], wherein the first cell membrane binding domain is a CD8α-derived transmembrane domain, or a CD28-derived transmembrane domain, and the second cell membrane binding domain is a CD8α-derived transmembrane domain, or a CD28-derived transmembrane domain.
[10] The polynucleotide according to any one of [1] to [5] and [9], wherein the extracellular domain and the first cell membrane binding domain are linked to each other via a CD8α-derived linker peptide, or a CD28-derived linker peptide, and the intracellular domain and the second cell membrane binding domain are linked to each other via a CD8α-derived linker peptide, or a CD28-derived linker peptide.
[11] The polynucleotide according to any one of [1] to [5], [9] and [10], wherein the first polypeptide and the second polypeptide are linked to each other via a self-cleaving peptide.
[12] The polynucleotide according to [11], wherein the self-cleaving peptide is a self-cleaving peptide T2A.
[13] The polynucleotide according to any one of [1] to [5] and [9] to [12], wherein the second polypeptide has an extracellular domain containing an amino acid sequence derived from a ligand molecule of a receptor endogenously present in an immune cell into which the polynucleotide is to be genetically introduced.
[14] The polynucleotide according to [13], wherein the ligand molecule is IL7 or IL21.
[15] The polynucleotide according to any one of [1] to [14], further encoding a chimeric antigen receptor (CAR) containing a single chain antibody, a transmembrane domain, and an ITAM intracellular signaling region.
[16] A vector comprising a promoter, and the polynucleotide according to any one of [1] to [15] operably linked downstream of the promoter (which vector will be hereinafter sometimes referred to as a “present vector”).
[17] An immune cell into which the vector according to [16] has been introduced (which immune cell will be hereinafter sometimes referred to as a “present immune cell”).
[18] A medicament comprising the immune cell according to [17](which medicament will be hereinafter sometimes referred to as a “present medicament”).
[19] The medicament according to [18], wherein the immune cell is administered to a treatment or prevention subject within 10 days after gene introduction of the vector according to [16] to the immune cell.
[20] A polypeptide comprising:
an extracellular domain containing an amino acid sequence derived from a ligand protein that binds to a receptor of IL15, IL2, or IL7, wherein the binding of the ligand protein to the receptor on an immune cell transmits a signal similar to a binding signal of the cytokine into the immune cell via the receptor;
a transmembrane domain; and
an intracellular domain containing a region derived from an intracellular domain of a TNF receptor superfamily (TNFRSF) molecule, but not containing an ITAM (Immunoreceptor Tyrosine-based Activation Motif) intracellular signaling region (which polypeptide is, namely, a present membrane-bound cytokine-TNFRSF molecule chimeric ligand).
[21] A polypeptide combination of:
a first polypeptide containing: an extracellular domain containing an amino acid sequence derived from a ligand protein that binds to a receptor of IL15, IL2, or IL7, wherein the binding of the ligand protein to the receptor on an immune cell transmits a signal similar to a binding signal of the cytokine into the immune cell via the receptor; and a first cell membrane binding domain (which polypeptide will be hereinafter sometimes referred to as a “present first polypeptide”); and
a second polypeptide containing: a second cell membrane binding domain; and an intracellular domain containing a region derived from an intracellular domain of a TNF receptor superfamily (TNFRSF) molecule, but not containing an ITAM (Immunoreceptor Tyrosine-based Activation Motif) intracellular signaling region (which polypeptide will be hereinafter sometimes referred to as a “present second polypeptide”) (which combination is, namely, a present polypeptide combination) (hereinafter, a present membrane-bound cytokine-TNFRSF molecule chimeric ligand, and a present polypeptide combination are sometimes collectively referred to as a “present polypeptide”).
[22] The polypeptide according to [20] or [21], wherein the IL15, IL2, or IL7, and the TNFRSF molecule are derived from a human, a mouse, or a rat.
[23] The polypeptide according to any one of [20] to [22], wherein the extracellular domain containing an amino acid sequence derived from the ligand protein that binds to an IL15 receptor contains an amino acid sequence having at least 80% sequence identity with an amino acid sequence shown in SEQ ID NO: 22, and retains activity of binding to the IL15 receptor; the extracellular domain containing an amino acid sequence derived from the ligand protein that binds to an IL2 receptor contains an amino acid sequence having at least 80% sequence identity with an amino acid sequence shown in SEQ ID NO: 17, and retains activity of binding to the IL2 receptor; and the extracellular domain containing an amino acid sequence derived from the ligand protein that binds to an IL7 receptor contains an amino acid sequence having at least 80% sequence identity with an amino acid sequence shown in SEQ ID NO: 20, and retains activity of binding to the IL7 receptor.
[24] The polypeptide according to any one of [20] to [23], wherein the TNFRSF molecule is TNFR2, OX40, HVEM, CD27, or CD137.
[25] The polypeptide according to any one of [20] and [22] to [24], wherein the transmembrane domain is a CD8α-derived transmembrane domain, or a CD28-derived transmembrane domain.
[26] The polypeptide according to any one of [20] and [22] to [25], wherein the extracellular domain and the transmembrane domain are linked to each other via a linker peptide.
[27] The polypeptide according to [26], wherein the linker peptide is a CD8α-derived linker peptide, or a CD28-derived linker peptide.
[28] The polypeptide according to any one of [21] to [24], wherein the first cell membrane binding domain is a CD8α-derived transmembrane domain, or a CD28-derived transmembrane domain, and the second cell membrane binding domain is a CD8α-derived transmembrane domain, or a CD28-derived transmembrane domain.
[29] The polypeptide according to any one of [21] to [24] and [28], wherein the extracellular domain and the first cell membrane binding domain are linked to each other via a CD8α-derived linker peptide, or a CD28-derived linker peptide, and the intracellular domain and the second cell membrane binding domain are linked to each other via a CD8α-derived linker peptide, or a CD28-derived linker peptide.
[30] The polypeptide according to any one of [21] to [24], [28] and [29], wherein the first polypeptide and the second polypeptide are linked to each other via a self-cleaving peptide.
[31] The polypeptide according to [30], wherein the self-cleaving peptide is a self-cleaving peptide T2A.
[32] The polypeptide according to any one of [21] to [24] and [28] to [31], wherein the second polypeptide has an extracellular domain containing an amino acid sequence derived from a ligand molecule of a receptor endogenously present in an immune cell in which the polypeptide is to be expressed.
[33] The polypeptide according to [32], wherein the ligand molecule is IL7 or IL21.
[34] An immune cell wherein the polypeptide according to any one of [20] and [22] to [27] is expressed on its cell membrane (which immune cell will be hereinafter sometimes referred to as a “present immune cell (2-1)”).
[35] An immune cell wherein the first polypeptide and the second polypeptide of the polypeptide according to any one of [21] to [24] and [28] to [33] are expressed on its cell membrane (which immune cell will be hereinafter sometimes referred to as the “present immune cell (2-2)”).
[36] The immune cell according to [35], wherein
a vector carrying the first polypeptide and the second polypeptide, or
a vector carrying the first polypeptide and a vector carrying the second polypeptide are introduced.
[37] A method for preparing a cell population containing the immune cell according to any one of [17], and [34] to [36], comprising the following steps (A) and (B) of:
(A) conducting gene introduction of the vector according to [16] to the immune cell; and
(B) culturing the immune cell for 5 days or more after the gene introduction.
[38] The method according to [37],
wherein the immune cell resulting from the gene introduction is cultured in the step (B) in the absence of a ligand protein that binds to a receptor of IL15, IL2, or IL7, wherein the binding of the ligand protein to the receptor on an immune cell transmits a signal similar to a binding signal of the cytokine into the immune cell via the receptor.

Furthermore, examples of other embodiments of the present invention include a method for treating or preventing a disease including a step of administering a present immune cell (1), a present immune cell (2-1), or a present immune cell (2-2) (which cells will be hereinafter sometimes collectively referred to as a “present immune cell”) to a subject requiring treatment or prevention of the disease (which method will be hereinafter sometimes referred to as a “present treatment or prevention method”); a present immune cell for use for treating or preventing a disease; use of a present immune cell in production of a medicament; and a kit for improving cell proliferation of an immune cell including a present polynucleotide, a present vector, a present membrane-bound cytokine-TNFRSF molecule chimeric ligand, or a present polypeptide combination (which kit will be hereinafter sometimes referred to as a “present kit”).

### Advantageous Effects of Invention

According to the present invention, when a present cytokine extracellular domain and the present TNFRSF molecule intracellular domain are separately expressed on the cell membrane of an immune cell, or expressed on the cell membrane of an immune cell as an single polypeptide with these domains linked to each other, no matter whether it has been pre-cultured for several days before administration to the living body, and without depending on a donor from which the immune cell is derived, the immune cell can favorably survive and/or proliferate also in the living body in which a high concentration of cytokine cannot be expected, and in addition, the therapeutic effect of the immune cell for treatment such as a CAR-T cell can be improved.

### BRIEF DESCRIPTION OF DRAWINGS

FIG. 1A illustrates the results of flow cytometry analysis using an anti-CD3 antibody of samples sampled 3 days and 7 days after PBMC is stimulated with an anti-CD3 antibody, and infected with BFP expressing lentivirus. The left graph is a contour plot (X-axis: BFP, Y-axis: CD3), and the right graph illustrates a CD3 positive rate in a BFP positive cell group (rate of CD3 positive cell [T cell] expressed by BFP). FIG. 1B is a schematic diagram of an expression pattern of IL15TM and “IL15-IL15Rα”. FIG. 1C illustrates diagrams of results of flow cytometry analysis of the cell number obtained by culturing a BFP-expressing control T cell in 3 types of media (basal medium [−], IL2 medium [+IL2], and IL15 medium [+IL15]). FIG. 1D illustrates diagrams of results of flow cytometry analysis of the cell number obtained by culturing IL15TM expressing T cell and “IL15-IL15Rα” expressing T cell in a basal medium (−). In scatter plots (X-axis: day, Y-axis: change rate of cell number) of FIGS. 1C and 1D, respective symbols (□, ⋄, Δ, x, ◯, and +) indicate respective donors.

FIG. 2 illustrates diagrams of results of flow cytometry analysis of the cell number obtained by culturing, in a basal medium, a T cell expressing a molecule in which an intracellular domain derived from each of seven TNFRSF molecules (TNFR2, OX40, HVEM, CD27, CD137, CD30, or DR3), or an intracellular domain derived from each of two CD28 family molecules (CD28 or ICOS) is linked to membrane-bound IL15 (molecule of type (III) described below). In scatter plots (X-axis: day, Y-axis: change rate of cell number), respective symbols (□, ⋄, Δ, x, ◯, and +) indicate respective donors.

FIG. 3A is a schematic diagram illustrating types (I), (II), and (III) of the expression pattern of a cytokine-derived extracellular domain (shown as “CYTOKINE” in the drawing), and a TNFRSF molecule-derived intracellular domain (shown as “TNFRSF MOLECULE” in the drawing). Specifically, in the expression pattern of the type (I), a general cytokine not tethered to a cell membrane (hereinafter sometimes referred to as a “secreted cytokine”), and a TNFRSF molecule-derived intracellular domain are independently expressed, in the expression pattern of the type (II), a membrane-bound cytokine, and a TNFRSF molecule-derived intracellular domain are independently expressed, and in the expression pattern of the type (III), a membrane-bound cytokine and a TNFRSF molecule-derived intracellular domain are linked to each other to be expressed as a fusion protein (hereinafter sometimes referred to as a “membrane-bound cytokine-TNFRSF molecule chimeric ligand). FIG. 3B is a schematic diagram of constructs for expressing the molecules of the types (I) to (III). FIG. 3C illustrates diagrams of results of flow cytometry analysis of the cell number obtained by culturing, in a basal medium, T cells expressing IL15 as the cytokine of the types (I) to (III), and expressing five TNFRSF molecules (TNFR2, OX40, HVEM, CD27, and CD137) as the TNFRSF molecule. In scatter plots (X-axis: day, Y-axis: change rate of cell number), respective symbols (□, ⋄, Δ, X, and ◯) indicate respective donors. That secreted IL15 (soluble IL15: sIL15) or membrane-bound IL15 (transmembrane IL15: IL15TM), and a co-stimulatory molecule-derived intracellular domain are independently expressed is shown with “/”, and that these are linked to be expressed is shown with “−”.

FIG. 4 illustrates diagrams of results of flow cytometry analysis of the cell number obtained by culturing, in a basal medium, T cells expressing six cytokines except for IL15 (IL2, IL4, IL6, IL7, IL9, and IL21) as the cytokine of the expression pattern of the type (III). In scatter plots (X axis: day, Y axis: change rate of cell number), respective symbols (□, ⋄, Δ, x, ◯, and +) indicate respective donors.

FIG. 5A illustrates diagrams of results of flow cytometry analysis of the cell number obtained by culturing, in a basal medium, T cells expressing two types of IL15TM (IL15TM containing a CD8α-derived linker peptide and a transmembrane domain [“IL15TM CD8α LINKER/TM” in the drawing]; and IL15TM containing a CD28-derived linker peptide and a transmembrane domain [“IL15TM CD28 LINKER/TM” in the drawing], or T cells expressing two types of IL15TM-TNFR2 (IL15TM-TNFR2 containing a CD8α-derived linker peptide and a transmembrane domain [“IL15TM-TNFR2 CD8α LINKER/TM” in the drawing]; and IL15TM-TNFR2 containing a CD28-derived linker peptide and a transmembrane domain [“IL15TM-TNFR2 CD28 LINKER/TM” in the drawing]. FIG. 5B illustrates diagrams of results of flow cytometry analysis of the cell number obtained by culturing, in a basal medium, T cells expressing IL15TM-TNFR2 containing three types of CD8α-derived linker peptides (SEQ ID NO: 15 [“62aa” in the drawing], SEQ ID NO: 31 [“30aa” in the drawing”, and SEQ ID NO: 32 [“15aa” in the drawing]. In scatter plots (X axis: day, Y axis: change rate of cell number), respective symbols (□, ⋄, Δ, x, and ◯) indicate respective donors.

FIG. 6A is a schematic diagram of the expression pattern of type (IV) of a cytokine-derived extracellular domain (“CYTOKINE” in the drawing), and a TNFRSF molecule-derived intracellular domain (“TNFRSF MOLECULE” in the drawing). Specifically, a membrane-bound cytokine and a present membrane-bound ligand molecule-TNFRSF molecule chimeric ligand are co-expressed. FIG. 6B is a schematic diagram of a construct for expressing the molecule of the type (IV).

FIG. 6C illustrates, as scatter plots (X-axis: day, Y-axis: change rate of cell number), the results of flow cytometry analysis of the cell number obtained by culturing, in a basal medium, T cells expressing IL15 as the cytokine of the type (IV), expressing two cytokines (IL7 and IL21) as a ligand molecule, and expressing five TNFRSF molecules (TNFR2, OX40, HVEM, CD27, and CD137) as a TNFRSF molecule. As a comparison control, results obtained by similarly analyzing the expression pattern of the types (II) and (III) of these cytokine-derived extracellular domain and TNFRSF molecule-derived intracellular domain are shown together. In scatter plots, respective symbols (□, ⋄, Δ, *, x, ◯, and +) indicate respective donors. A membrane-bound cytokine and a TNFRSF molecule-derived intracellular domain are independently expressed is shown with “/”, and that these are linked to be expressed is shown with “−”.

FIG. 7A illustrates diagrams of results of flow cytometry analysis of the cell number of BFP-positive viable cells (the cell number of T cells expressing various molecules) contained in the spleen of each of two mice (NOG mouse and NOG-ΔMHC mouse) transplanted with a BFP-expressing control T cell (“BFP” in the drawing), an IL15TM expressing T cell (“IL15TM” in the drawing), and two present membrane-bound cytokine-TNFRSF molecule chimeric ligands (“IL15TM-CD137” and “IL15TM-HVEM”), and counted 10 days after the transplantation. FIG. 7B illustrates a diagram of results of flow cytometry analysis of the cell number of BFP-positive viable cells (the cell number of T cells expressing various molecules) contained in the spleen of each NOG mouse transplanted with a BFP-expressing control T cell (“BFP” in the drawing”), a conventional membrane-bound IL15-expressing T cell (“IL15TM” in the drawing), and a T cell expressing a present membrane-bound cytokine-TNFRSF molecule chimeric ligand (“IL15TM-HVEM”), and counted 10 days after the transplantation. Respective symbols (□, ⋄, and ◯) used in the drawings indicate types of donors of the T cell (PBMC).

FIG. 8A is a schematic diagram of “CAR (CD137-CD3ζ)” (CAR using CD137 as a co-stimulatory molecule), and “CAR (CD3ζ)/IL15TM-CD137” (co-expression of CAR, and a chimeric ligand containing IL15-TM and CD137-derived intracellular domain). FIG. 8B is a schematic diagram of constructs for expressing “CAR (CD137-CD3ζ)” and “CAR (CD3ζ)/IL15TM-CD137”. FIG. 8C illustrates the outline of an evaluation system for cytotoxic activity of a CAR-T cell against two cancer cell lines (RAJI cell line, and CD19+HeLa cell line). Specifically, the two cancer cell lines are added to CAR-T cells (“FIRST ADDITION OF CANCER CELL” in the drawing), flow cytometry analysis is performed 3 days after (“FCM” in the drawing), and then the cancer cells are added again (“SECOND ADDITION OF CANCER CELL” in the drawing), and flow cytometry analysis is performed after 7, 12, and 14 days. FIG. 8D illustrates diagrams of analysis results of cytotoxic activity, against the two cancer cell lines, of six CARs (“CAR (CD137-CD3ζ)”, “CAR (CD3ζ)/IL15TM-CD137”, “CAR [CD3ζ]/IL15TM-TNFR2”, “CAR [CD3ζ]/IL15TM-OX40”, “CAR [CD3ζ]/IL15TM-CD27”, and “CAR [CD3ζ]/IL15TM-HVEM”), and a BFP-expressing control T cell (“BFP” in the drawing). Respective symbols (□, Δ, and ◯) used in the drawings indicate respective donors. FIG. 8E illustrates diagrams, shown as scatter plots (X-axis: day, Y-axis: change rate of cell number), of the analysis results of a change rate of the cell number of six CAR-T cells and BFP-expressing control T cell (“BFP” in the drawing) in an evaluation system for cytotoxic activity.

FIG. 9 is a diagram illustrating the results of analysis in which a luciferase-expressing CD19+HeLa cell is transplanted to cancer-bearing model mice, two CAR-T cells (a “CAR (CD137-CD3ζ)”-expressing T cell and a “Car (CD3ζ)/IL15TM-OX40”-expressing T cell) are administered to the mice, and presence level of luciferase-expressing CD19+HeLa cell in each mouse is analyzed by using the level of luciferin-derived luminescence as an index.

FIG. 10 illustrates diagrams of the results of flow cytometry analysis of the cell number obtained in a “CAR (CD137-CD3ζ)” expressing T cell (“CAR (CD137-CD3ζ)”) cultured in IL2 medium (+IL2), and CAR-T cells expressing three present membrane-bound cytokine-TNFRSF molecule chimeric ligands (“IL15TM-CD137”, “IL15TM-OX40”, and “IL15TM-CD27”) cultured in basal medium (−) (“CAR (CD3ζ)/IL15TM-CD137”, “CAR (CD3ζ)/IL15TM-OX40”, and “CAR (CD3ζ)/IL15TM-CD27”). Upper scatter plots indicate the change rate of the cell number, and lower scatter plots indicate the rate of the cell number of BFP-positive viable cells (number of T cells expressing various molecules). Respective symbols (□, ⋄, Δ, x, and ◯) indicate respective donors.

FIG. 11 illustrates diagrams of the results of flow cytometry analysis obtained by transplanting, to a NOG mouse, a BFP-expressing control T cell (“BFP” in the drawing), and T cells expressing two present membrane-bound cytokine-TNFRSF molecule chimeric ligands (“IL15TM-OX40”) without pre-culture, and analyzing the cell number of BFP-positive viable cells (“IL15TM-OX40” expressing T cell number) contained in the spleen of the mouse counted after 9 days.

FIG. 12 illustrates diagrams of results of flow cytometry analysis of the cell number obtained by culturing, in a basal medium, a GFP-expressing control NK cell (“GFP” in the drawing), an IL15TM expressing NK cell, and NK cells expressing five present membrane-bound cytokine-TNFRSF molecule chimeric ligands (“IL15TM-OX40”, “IL15TM-TNFR2”, “IL15TM-HVEM”, “IL15TM-CD137”, and “IL15TM-CD27”). In scatter plots (X axis: day, Y axis: change rate of cell number), respective symbols (□, ⋄, Δ, x, −, ◯, and +) indicate respective donors.

### DESCRIPTION OF EMBODIMENTS

The present invention provides, as a present polynucleotide, a polynucleotide comprising: a polynucleotide encoding an extracellular domain containing an amino acid sequence derived from a ligand protein that binds to a receptor of IL15, IL2, or IL7, wherein the binding of the ligand protein to the receptor on an immune cell transmits a signal similar to a binding signal of the cytokine into the immune cell via the receptor (which ligand protein is, namely, a present cytokine) (which extracellular domain is, namely, a present cytokine extracellular domain) (which polynucleotide is, namely, a present cytokine extracellular domain encoding region); and a polynucleotide encoding an intracellular domain containing a region derived from an intracellular domain of a TNFRSF molecule, but not containing an ITAM intracellular signaling region (which intracellular domain is, namely, a present TNFRSF molecule intracellular domain) (which polynucleotide is, namely, a present TNFRSF intracellular domain encoding region).

One aspect of the present polynucleotide is (a) a polynucleotide designed to be an extracellular domain-intracellular domain-containing molecule gene (which gene is, namely, a “present membrane-bound cytokine-TNFRSF molecule chimeric ligand encoding region”) that expresses, on an immune cell, a polypeptide containing both of a present cytokine extracellular domain and a present TNFRSF molecule intracellular domain (which polypeptide is, namely, a “present membrane-bound cytokine-TNFRSF molecule chimeric ligand”) wherein the extracellular domain and the intracellular domain are linked to each other via a transmembrane domain.

Another aspect of the present polynucleotide is (b) a polynucleotide designed to be an extracellular domain-containing molecule gene and an intracellular domain-containing molecule gene (which genes are, namely, present polypeptide combination encoding regions) that express separate polypeptides as a polypeptide combination on an immune cell, wherein the first polypeptide containing a present cytokine extracellular domain and the second polypeptide containing a present TNFRSF molecule intracellular domain (which polypeptide combination is, namely, a “present polypeptide combination”) wherein the extracellular domain and the intracellular domain are linked to a first cell membrane binding domain and a second cell membrane binding domain, respectively.

The present polynucleotide having the characteristic (a) described above (namely, the present membrane-bound cytokine-TNFRSF molecule chimeric ligand encoding region [in other words, a polynucleotide encoding the present membrane-bound cytokine-TNFRSF molecule chimeric ligand]) is a polynucleotide containing a present cytokine extracellular domain encoding region, a transmembrane domain encoding region, and a present TNFRSF molecule intracellular domain encoding region, and designed to be expressed in an immune cell as a present membrane-bound cytokine-TNFRSF molecule chimeric ligand when the present cytokine extracellular domain, and the present TNFRSF molecule intracellular domain are linked to each other via the transmembrane domain.

Besides, the present polynucleotide having the characteristic (b) described above (namely, the present polypeptide combination encoding region [in other words, the polynucleotide encoding the present polypeptide combination]) is a polynucleotide containing following two polynucleotides: a polynucleotide containing a present cytokine extracellular domain encoding region, and a first cell membrane binding domain encoding region (which polynucleotide will be hereinafter sometimes referred to as a “present first polypeptide encoding region”); and a polynucleotide containing a second cell membrane binding domain encoding region and a present TNFRSF molecule intracellular domain encoding region (which polynucleotide will be hereinafter sometimes referred to as a “present second polypeptide encoding region”), and designed to enable expression of a polypeptide combination in an immune cell of the present first polypeptide and the present second polypeptide.

### 1. Description of Terms

Herein, the term “cytokine” means a polypeptide having a low molecular weight (usually in the range of 10,000 to 30,000, and preferably from 15,000 to 30,000) that is secreted from an immune cell, and is involved in one or more functions selected from differentiation, maturation, activation, and proliferation of an immune cell. Examples of the cytokine include IL2, IL4, IL6, IL7, IL9, IL15, IL21, IL1α, IL1β, IL18, IL33, IL36, IL37, and IL38.

Herein, the term “present cytokine” means a ligand protein that binds to a receptor of any one cytokine selected from the group consisting of IL2, IL7, and IL15, a signal similar to a binding signal of the cytokine being transmitted, through a bond between the ligand protein and the receptor on an immune cell, into an immune cell via the receptor. The present cytokine is typically naturally occurring IL2, IL7, or IL15. Besides, the present cytokine is not limited to a naturally occurring cytokine, but a mutant retaining the function (for example, mutant IL2/IL15 [SEQ ID NO: 56] described in the literature “Nature. 2019 January; 565 (7738): 186-191 doi: 10.1038/s41586-018-0830-7”) can be used. In another aspect of the present cytokine, a protein containing a monoclonal antibody having agonist activity for an IL2 receptor, an IL7 receptor, or an IL15 receptor, or containing an antigen binding fragment, such as a scFv (single chain Fv), or Fab, designed by using the complementarity determining region (CDR) sequence thereof can be used. An anti-IL15 receptor scFv simulating the function of IL15 is described in, for example, the literature “Cell. 2022 Apr. 14; 185 (8): 1414-1430.e19 doi: 10.1016/j.cell.2022.02.025.”.

Herein, the term “membrane-bound cytokine” means a chimeric peptide artificially designed by linking, to a transmembrane domain, or a cell membrane binding domain, an amino acid sequence derived from a natural secreted cytokine, or a ligand protein simulating the function thereof with the binding activity of the cytokine to a receptor retained, and is herein referred to as “cytokine TM” in some cases. In using a secreted cytokine, the amino acid sequence is not limited to that of a naturally occurring cytokine, but several (for example, 10 or less, 7 or less, 5 or less, 4 or less, 3 or less, 2 or less, or 1) amino acids may be substituted, deleted, inserted, and/or added in the amino acid sequence of a naturally occurring or mutant cytokine as long as the binding activity to a receptor is retained. Such a change of the amino acid is caused, in the amino acid sequence of the cytokine, preferably in a position excluding an amino acid having been reported to be significant for the bond to a receptor. Regarding IL2, as an amino acid residue significant for the bond to an IL2 receptor a subunit, K35, T37, R38, T41, F42, K43, F44, Y45, E61, E62, K64, P65, E68, L72, and Y107 (corresponding to amino acid residues at positions 35, 37, 38, 41, 42, 43, 44, 45, 61, 62, 64, 65, 68, 72, and 107, respectively in SEQ ID NO: 17) have been reported. As an amino acid residue significant for the bond to an IL2 receptor β subunit, L19, D20, M23, R81, D84, S87, and N88 (corresponding to amino acid residues at positions 19, 20, 23, 81, 84, 87, and 88, respectively in SEQ ID NO: 17) have been reported. As an amino acid residue significant for the bond to a γc subunit, E15, L18, Q22, N119, T123, Q126, S127, 1129, S130, and T133 (corresponding to amino acid residues at positions 15, 18, 22, 119, 123, 126, 127, 129, 130, and 133, respectively in SEQ ID NO: 17) have been reported (the literature “Proc Natl Acad Sci USA. 2006 Feb. 21; 103(8): 2788-93. doi: 10.1073/pnas.0511161103.”, and the literature “Nat Immunol. 2012 December; 13(12): 1187-95. doi: 10.1038/ni.2449.”). Besides, regarding IL7, an amino acid residue significant for the bond to an IL7 receptor a subunit, K10, Q1l, S14, V15, L16, V18, S19, Q22, S71, T72, D74, L77, H78, L80, K81, E84, G85, 188, and L89 (corresponding to amino acid residues at positions 10, 11, 14, 15, 16, 18, 19, 22, 71, 72, 74, 77, 78, 80, 81, 84, 85, 88, and 89, respectively in SEQ ID NO: 20) have been reported. As an amino acid residue significant for the bond to a γc subunit, C47, R133, Q136, E137, K139, T140, C141, N143, and K144 (corresponding to amino acid residues at positions 47, 133, 136, 137, 139, 140, 141, 143, and 144, respectively in SEQ ID NO: 20) have been reported (the literature “Structure. 2009 Jan. 14; 17(1): 54-65. doi: 10.1016/j.str.2008.10.019.”). Regarding IL15, as an amino acid residue significant for the bond to an IL15 receptor a subunit, D22, A23, Y26, E46, V49, E53, E87, E89, and E90 (corresponding to amino acid residues at positions 22, 23, 26, 46, 49, 53, 87, 89, and 90, respectively in SEQ ID NO: 22) have been reported. As an amino acid residue significant for the bond to an IL15 receptor β subunit, S7, D8, K10, K11, D61, E64, and N65 (corresponding to amino acid residues at positions 7, 8, 10, 11, 61, 64, and 65, respectively in SEQ ID NO: 22) have been reported. As an amino acid residue significant for the bond to a γc subunit, K10, 529, D30, H32, H105, Q108, M109, I111, and N112 (corresponding to amino acid residues at positions 10, 29, 30, 32, 105, 108, 109, 111, and 112, respectively in SEQ ID NO: 22) have been reported (the literature “Nat Immunol. 2007 September; 8(9): 1001-7. doi: 10.1038/ni1492.”, and the literature “Nat Immunol. 2012 December; 13(12): 1187-95. doi: 10.1038/ni.2449.”). In the design of a membrane-bound cytokine of the present cytokine, an amino acid sequence in which mutation is introduced into an amino acid residue different from these amino acid residues significant for the binding to the receptors can be used. Besides, there is no need to use the full length of the amino acid sequence of a naturally occurring or mutant cytokine, and a part (for example, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, 2% or less, or 1% or less) of the N terminal and/or the C terminal may be deleted as long as the binding activity to a receptor is maintained.

Herein, “chimeric polypeptide” means a polypeptide obtained by fusing (linking) polypeptides derived from 2 or more different proteins by genetic manipulation. A chimeric polypeptide containing an amino acid sequence or a domain structure derived from a ligand that binds to an endogenous receptor (such as a cytokine, a growth factor, or a bioactive peptide) is designated as a chimeric ligand in some cases. In a present membrane-bound cytokine-TNFRSF molecule chimeric ligand, a present cytokine extracellular domain, and a transmembrane domain or a present TNFRSF molecule intracellular domain are in a chimeric relationship, and in a present first polypeptide, a present cytokine extracellular domain and a first cell membrane binding domain are in a chimeric relationship.

In the description of a chimeric polypeptide herein, it is described with “-” provided between the names of origin molecules. Regarding a membrane protein, label for an extracellular domain is provided on the left hand side, and an intracellular domain is provided on the right hand side. For example, a chimeric peptide in which membrane-bound IL15 and an intracellular domain of TNFR2 are linked is described as “IL15TM-TNFR2”. Besides, when different proteins are co-expressed in the same cell, “/” is provided between the names of molecules. For example, a present second polypeptide containing membrane-bound IL15 and a TNFR2-derived intracellular domain is described as “IL15TM/TNFR2”. When only the name of a TNFRSF molecule or a co-stimulatory molecule is shown as the molecule name, it means a chimeric peptide of a transmembrane domain or a cell membrane-binding domain, and a TNFRSF molecule or a co-stimulatory molecule that does not contain a characteristic extracellular domain, and optionally has a signal peptide, a tag peptide, a linker peptide, a spacer, or the like present outside the cell.

Species from which each domain/region contained in a present polynucleotide or a present polypeptide, or an immune cell is derived is not especially limited as long as it is a mammal, and examples include rodents such as a mouse, a rat, a hamster, and a guinea pig; lagomorphs such as a rabbit; ungulates such as a pig, a cow, a goat, a horse, and sheep; carnivores such as a dog, and a cat; and primates such as a human, a monkey, a rhesus monkey, a cynomolgus monkey, a marmoset, an orangutan, and a chimpanzee, and a human, a mouse, and a rat are preferred.

Herein, “TNFRSF molecule” refers to a receptor molecule classified into the TNF receptor superfamily, and has been reported to play a role in signaling related to maintenance/proliferation of an immune cell (Annu Rev Immunol. 2005; 23: 23-68. doi: 10.1146/annurev.immunol.23.021704.115839.). Examples of the TNFRSF molecule include TNFR1, NGFR, FAS, BCMA, CD137 (4-1BB), CD134 (OX40), HVEM, CD27, TNFR2, CD30, DR3, GITR, and LTβR. Among these, CD137 (4-1BB), CD134 (OX40), HVEM, CD27, TNFR2, CD30, DR3, GITR, and LTβR are also known as a molecule making a contribution to maintenance/proliferation of a co-stimulatory molecule or an immune cell. In the description of a chimeric polypeptide herein, when the name of a TNFRSF molecule is described, an amino acid sequence derived from an intracellular domain of the TNFRSF molecule is contained in an intracellular domain of the chimeric polypeptide unless otherwise mentioned.

Herein, “ITAM intracellular signaling region” means an intracellular domain containing a tyrosine phosphorylation motif (Immunoreceptor Tyrosine-based Activation Motif; ITAM) significant in an activation signaling cascade in an immune cell caused by a target antigen bond of the immune cell. Examples of the ITAM intracellular signaling region include intracellular domains such as CD3ζ, CD3δ, and FcRγ. In a T cell, a binding signal of an antigen presented in an MIC (MHC class I-related chain) and a TCR is transmitted into a cell via a TCR/CD3 complex. In an NK cell, a binding signal of an antibody bound to an antigen and an Fc receptor causes activation of a cell via the ITAM signaling region of the Fc receptor. Such an activation signal of an immune cell via an ITAM is designated as “ITAM-dependent activation signal”, and is significant for cytotoxic activity for a target cell, but is known to exhaust or/and damage the immune cell itself.

Herein, “extracellular domain” refers to a domain usually present mostly extracellularly when a membrane protein is expressed on the cell, and there is no need for all amino acid residues contained in the domain to present extracellularly, but a structure in which a part on the C terminal side of the amino acid sequence corresponding to the extracellular domain is embedded in the cell membrane may be employed in some cases depending on the amino acid sequence to be used and an amino acid sequence therearound.

Herein, “transmembrane domain” refers to a domain usually present mostly in the cell membrane when a membrane protein is expressed on the cell. There is no need for all amino acid residues contained in the domain to present in the cell membrane, but a structure in which a part of the N terminal of the amino acid sequence corresponding to the transmembrane domain is present extracellularly, and/or a part on the C terminal side of the amino acid sequence corresponding to the transmembrane domain is present intracellularly may be employed in some cases depending on the amino acid sequence to be used and an amino acid sequence therearound.

Herein, “intracellular domain” refers to a domain usually present mostly intracellularly when a membrane protein is expressed on the cell, and there is no need for all amino acid residues contained in the domain to present intracellularly, but a structure in which a part on the N terminal side of the amino acid sequence corresponding to the intracellular domain is embedded in the cell membrane may be employed in some cases depending on the amino acid sequence to be used and an amino acid sequence therearound.

Herein, “amino acid sequence derived from” a given protein or a partial domain thereof refers to an amino acid sequence containing an amino acid sequence that has at least 80% sequence identity with amino acids of the original natural protein, and retains at least a part of the function/activity of the natural protein or the partial domain thereof when expressed as a polypeptide.

Herein, “at least 80% sequence identity” means that the sequence identity with the entire target sequence is 80% or more, and means the identity of preferably 85% or more, more preferably 88% or more, further preferably 90% or more, still further preferably 93% or more, particular preferably 95% or more, further particularly preferably 98% or more, and most preferably 100%.

Herein, the term “sequence identity” means the degree of approximation of a polynucleotide sequence or an amino acid sequence (which is determined by matching between a query sequence and another sequence preferably of the same type (nucleic acid or protein sequence)). Examples of a preferable computer program method for calculating and determining the “sequence identity” include, but are not limited to, GCG BLAST (Basic Local Alignment Search Tool) (Altschul et al., J. Mol. Biol. 1990, 215:403-410; Altschul et al., Nucleic Acids Res. 1997, 25:3389-3402; Devereux et al., Nucleic Acid Res. 1984, 12:387), BLASTN 2.0 (Gish W., http://blast.wustl.edu, 1996-2002), FASTA (Pearson and Lipman, Proc. Natl. Acad. Sci. USA 1988, 85: 2444-2448), and GCG GelMerge for determining and aligning a pair of contigs overlapping in the largest length (Wibur and Lipman, SIAMJ. Appl. Math. 1984, 44: 557-567; Needleman and Wunsch, J. Mol. Biol. 1970, 48: 443-453).

Herein, “to have at least 80% sequence identity with an amino acid sequence shown in SEQ ID NO: X (or of SEQ ID NO: X)” means, in other words, that it has an amino acid sequence obtained by deleting, substituting, inserting, and/or adding 0, 1, or several amino acid residues in the amino acid sequence of SEQ ID NO: X, and has an equivalent function to a polypeptide consisting of the amino acid sequence of SEQ ID NO: X. Here, “amino acid sequence obtained by deleting, substituting, inserting, and/or adding 1, or several amino acid residues” means an amino acid sequence in which 1 to 30, preferably 1 to 20, more preferably 1 to 15, further preferably 1 to 10, still further preferably 1 to 5, still further preferably 1 to 3, and still further preferably 1 to 2 amino acid residues are deleted, substituted, inserted, and/or added. Besides, herein, “to have at least 80% sequence identity with a nucleotide sequence of SEQ ID NO: X” means, in other words, it has a nucleotide sequence obtained by deleting, substituting, inserting, and/or adding 0, 1, or several nucleotide residues in the nucleotide sequence of SEQ ID NO: X, and has an equivalent function to a polynucleotide consisting of the nucleotide sequence of SEQ ID NO: X. Here, “nucleotide sequence obtained by deleting, substituting, inserting, and/or adding 1, or several nucleotide residues” means a nucleotide sequence in which 1 to 30, preferably 1 to 20, more preferably 1 to 15, further preferably 1 to 10, still further preferably 1 to 5, still further preferably 1 to 3, and still further preferably 1 to 2 nucleotide residues are deleted, substituted, inserted, and/or added. These mutation treatments of amino acid residues and nucleotide residues can be conducted by an arbitrary method known to those skilled in the art such as chemical synthesis, a genetic engineering method, or mutagenesis.

Herein, “immune cell” means a cell that performs an immune function in a living body, or a cell that has been artificially induced from a stem cell to a cell having the equivalent function to such a cell. Examples of the immune cell include lymphocytic cells such as a T cell, an NK cell, and a B cell, antigen presenting cells such as a monocyte, a macrophage, and a dendritic cell, and granulocytes such as a neutrophil, an eosinophil, a basophil, and a mast cell. Examples of the T cell include an alpha beta T cell, a gamma delta T cell, a CD8⁺ T cell, CD4⁺ T cell, a tumor infiltrating T cell, a memory T cell, a naive T cell, and an NK T cell. A differentiation induction method from a stem cell to an immune cell, such as differentiation induction from an iPS cell to a T cell or to an NK cell, has been reported in the literature “Nat Biotechnol 2014, 32: 554-561” and the like.

The immune cell encompasses an immune cell in which a peptide/polypeptide for treatment or prevention is expressed by introducing, into a T cell derived from a living body, a polynucleotide encoding at least one peptide/polypeptide for treatment or prevention, or at least one peptide/polypeptide for treatment or prevention by employing genetic modification technology, and an immune cell differentiation-induced from an ES cell or an iPS cell.

Herein, “peptide/polypeptide for treatment or prevention” means a naturally derived protein (polypeptide) or an artificially designed protein having a therapeutic effect and/or a preventive effect against a disease, a peptide contained in such a protein, a variant retaining the biological function of such a protein or peptide, and the like. The naturally derived protein or peptide is not especially limited as long as it is a protein, a peptide or the like that is endogenously held in a human, and is known to accelerate onset or progression of a medical condition when reduced in expression level, or to improve the medical condition or slow the progression when increased in expression level or replenished. Examples of the artificially designed protein or peptide include a monoclonal antibody used in an antibody preparation, a chimeric antigen receptor used in CAR-T cell therapy, a chimeric autoantibody receptor used in CAAR-T cell therapy employed for an autoimmune disease, and a ligand peptide artificially designed based on binding activity to a natural receptor.

### 2. Structures of Present Polypeptide

The present invention provides a present polypeptide containing domains, partial structures, and the like described below, and a polynucleotide encoding the present polypeptide (namely, a present polynucleotide).

A present cytokine extracellular domain contained in the present polypeptide is an extracellular domain containing an amino acid sequence derived from a present cytokine, and here, “amino acid sequence derived from a present cytokine” means an amino acid sequence of the present cytokine that is a natural (wild type) secreted cytokine, or an amino acid sequence of a variant (mutant) having receptor binding ability equivalent to the present cytokine that is a natural secreted cytokine. An example of such a variant includes the mutant IL2/IL15 [SEQ ID NO: 56] described in the literature “Nature. 2019 January; 565 (7738): 186-191. doi: 10.1038/s41586-018-0830-7”) described above.

Species from which the present cytokine is derived is not especially limited as long as it is a mammal, and examples include a human, a mouse, a rat, a monkey, a dog, a rabbit, and a pig, and a human, a mouse, and a rat are preferred, and a human is more preferred. In description of the respective structural regions of the present invention including the present cytokine, when an origin species is not particularly described, it means that the region/amino acid sequence is derived from a human molecule.

Examples of the present cytokine extracellular domain includes:
a polypeptide containing an amino acid sequence having at least 80% sequence identity with the amino acid sequence of human IL15 (SEQ ID NO: 22), the amino acid sequence of mouse IL15 (SEQ ID NO: 33), or the amino acid sequence of rat IL15 (SEQ ID NO: 34), and retaining activity of binding to a human, mouse, or rat IL15 receptor;
a polypeptide containing an amino acid sequence having at least 80% sequence identity with the amino acid sequence of human IL15 (SEQ ID NO: 22), having an amino acid sequence retaining 1 or several (for example, 20, 19, 18, 17, 16, 15, 14, 13, 12, 11, 10, 9, 8, 7, 6, 5, 4, 3, or 2) amino acid residues selected from the group consisting of S7, D8, K10, K11, D22, A23, Y26, S29, D30, H32, E46, V49, E53, D61, E64, N65, E87, E89, E90, H105, Q108, M109, I111, and N112, which have been reported to be significant for the bond to the receptor, and retaining activity of binding to a human IL15 receptor, the polypeptide preferably retaining all the amino acid residues significant for the bond to the receptor;
a polypeptide containing an amino acid sequence having at least 80% sequence identity with the amino acid sequence of human IL2 (SEQ ID NO: 17), the amino acid sequence of mouse IL2 (SEQ ID NO: 35), or the amino acid sequence of rat IL2 (SEQ ID NO: 36), and retaining activity of binding to a human, mouse, or rat IL2 receptor;
a polypeptide containing an amino acid sequence having at least 80% sequence identity with the amino acid sequence of human IL2 (SEQ ID NO: 17), having an amino acid sequence retaining 1 or several (for example, 20, 19, 18, 17, 16, 15, 14, 13, 12, 11, 10, 9, 8, 7, 6, 5, 4, 3, or 2) amino acid residues selected from the group consisting of E15, L18, L19, D20, Q22, M23, K35, T37, R38, T41, F42, K43, F44, Y45, E61, E62, K64, P65, E68, L72, R81, D84, 587, N88, Y107, N119, T123, Q126, 5127, 1129, 5130, and T133, which have been reported to be significant for the bond to the receptor, and retaining activity of binding to a human IL12 receptor, the polypeptide preferably retaining all the amino acid residues significant for the bond to the receptor;
a polypeptide containing an amino acid sequence having at least 80% sequence identity with the amino acid sequence of human IL7 (SEQ ID NO: 20), the amino acid sequence of mouse IL7 (SEQ ID NO: 37), or the amino acid sequence of rat IL7 (SEQ ID NO: 38), and retaining activity of binding to a human, mouse, or rat IL7 receptor; and
a polypeptide containing an amino acid sequence having at least 80% sequence identity with the amino acid sequence of human IL7 (SEQ ID NO: 20), having an amino acid sequence retaining 1 or several (for example, 20, 19, 18, 17, 16, 15, 14, 13, 12, 11, 10, 9, 8, 7, 6, 5, 4, 3, or 2) amino acid residues selected from the group consisting of K10, Q1l, S14, V15, L16, V18, S19, Q22, C47, S71, T72, D74, L77, H78, L80, K81, E84, G85, 188, L89, R133, Q136, E137, K139, T140, C141, N143, and K144, which have been reported to be significant for the bond to the receptor, and retaining activity of binding to a human IL7 receptor, the polypeptide preferably retaining all the amino acid residues significant for the bond to the receptor.

A present TNFRSF molecule intracellular domain contained in a present polypeptide is an intracellular domain containing a region derived from an intracellular domain of a TNFRSF molecule, but not containing an ITAM intracellular signaling region. Here, “region derived from an intracellular domain of a TNFRSF molecule” means a polypeptide that is an intracellular domain or a part thereof of a receptor molecule classified into TNFRSF, and retains intracellular signaling activity of the TNFRSF molecule. Such a polypeptide may contain an amino acid sequence of an intracellular domain of a natural (wild type) TNFRSF molecule, or an amino acid sequence of a variant (mutant) having a function equivalent to that of an intracellular domain of a natural TNFRSF molecule.

Examples of the TNFRSF molecule include TNFR1, NGFR, FAS, BCMA, CD137 (4-1BB), CD134 (OX40), HVEM, CD27, TNFR2, CD30, DR3, GITR, and LTβR. Since the TNFRSF molecule is also known as a molecule contributing to maintenance/proliferation of a co-stimulatory molecule or an immune cell, CD137 (4-1BB), CD134 (OX40), HVEM, CD27, TNFR2, CD30, DR3, GITR, and LTβR are preferred. Since the effect has been verified as described in Examples below, preferable examples include TNFRSF molecules selected from TNFR2, OX40, HVEM, CD27, and CD137.

Examples of the “region derived from an intracellular domain of a TNFRSF molecule” include:
a polypeptide containing an amino acid sequence having at least 80% sequence identity with the amino acid sequence of an intracellular domain of human TNFR2 (SEQ ID NO: 5), the amino acid sequence of an intracellular domain of mouse TNFR2 (SEQ ID NO: 39), or the amino acid sequence of an intracellular domain of rat TNFR2 (SEQ ID NO: 40);
a polypeptide containing an amino acid sequence having at least 80% sequence identity with the amino acid sequence of an intracellular domain of human OX40 (SEQ ID NO: 6), the amino acid sequence of an intracellular domain of mouse OX40 (SEQ ID NO: 41), or the amino acid sequence of an intracellular domain of rat OX40 (SEQ ID NO: 42);
a polypeptide containing an amino acid sequence having at least 80% sequence identity with the amino acid sequence of an intracellular domain of human HVEM (SEQ ID NO: 7), the amino acid sequence of an intracellular domain of mouse HVEM (SEQ ID NO: 43), or the amino acid sequence of an intracellular domain of rat HVEM (SEQ ID NO: 44);
a polypeptide containing an amino acid sequence having at least 80% sequence identity with the amino acid sequence of an intracellular domain of human CD27 (SEQ ID NO: 8), the amino acid sequence of an intracellular domain of mouse CD27 (SEQ ID NO: 45), or the amino acid sequence of an intracellular domain of rat CD27 (SEQ ID NO: 46); and
a polypeptide containing an amino acid sequence having at least 80% sequence identity with the amino acid sequence of an intracellular domain of human CD137 (SEQ ID NO: 9), the amino acid sequence of an intracellular domain of mouse CD137 (SEQ ID NO: 47), or the amino acid sequence of an intracellular domain of rat CD137 (SEQ ID NO: 48).

A transmembrane domain used in a present polypeptide may be any polypeptide as long as it can penetrate a cell membrane, and may be a transmembrane domain derived from a natural receptor protein, or an artificially designed transmembrane domain not present in nature. Here, examples of the transmembrane domain derived from a natural receptor protein include transmembrane domains derived from various known receptor proteins, and a transmembrane domain derived from a receptor protein expressed on an immune cell is preferred. Specific examples include transmembrane domains derived from the TNFRSF molecule, a T cell receptor α or β chain, CD3ζ chain, CD28, CD3ε, CD45, CD4, CD5, CD8α, CD8β, CD9, CD16, CD22, CD33, CD27, CD64, CD80, CD86, CD134, CD137, ICOS, CD154, and GITR. Since the effect has been verified in Examples below, preferable examples include a CD8α-derived transmembrane domain, and a CD28-derived transmembrane domain.

A specific example of the CD8α-derived transmembrane domain includes a polypeptide containing an amino acid sequence having at least 80% sequence identity with the amino acid sequence of a human CD8α-derived transmembrane domain (SEQ ID NO: 16). A specific example of the CD28-derived transmembrane domain includes a polypeptide containing an amino acid sequence having at least 80% sequence identity with the amino acid sequence of a human CD28-derived transmembrane domain (SEQ ID NO: 30).

The respective domains contained in the present polypeptide may be linked to each other via a linker peptide and/or a spacer. A specific insertion site of the linker peptide or the spacer can be one or more sites selected from the N terminals of a present membrane-bound cytokine-TNFRSF molecule chimeric ligand, a present first polypeptide, and a present second polypeptide; the C terminals of these polypeptides; between a present cytokine extracellular domain and a transmembrane domain; between a transmembrane domain and a present TNFRSF molecule intracellular domain; between a present cytokine extracellular domain and a first cell membrane binding domain; and between a present TNFRSF molecule intracellular domain and a second cell membrane binding domain.

The linker peptide and the spacer are not especially limited as long as they do not inhibit the function of the present polypeptide. The length of the linker peptide is, for example, 3 to 300 amino acid residues (for example, 4 to 200 amino acid residues, 5 to 150 amino acid residues, 6 to 100 amino acid residues, 10 to 100 amino acid residues, or 15 to 62 amino acid residues). Examples of the linker peptide include a linker peptide derived from a protein present in nature (such as a CD8α-derived linker peptide, a CD8β-derived linker peptide, or a CD28-derived linker peptide), a peptide derived from a hinge region (for example, a CD8α-derived hinge region; a CD8β-derived hinge region; a CD28-derived hinge region; or a hinge region derived from various IgGs such as IgG2 [Longe hinge]), or IgG3 [Short hinge], and an artificially synthesized linker peptide (such as a flexible linker peptide), and a linker peptide selected from a CD8α-derived linker peptide, and a CD28-derived linker peptide is preferred. A specific example of the CD8α-derived linker peptide includes a polypeptide having at least 80% sequence identity with the amino acid sequence of any of SEQ ID NOs: 15, 31, and 32, a specific example the CD28-derived linker peptide includes a polypeptide having at least 80% sequence identity with the amino acid sequence of SEQ ID NO: 29, and a specific example of the flexible linker peptide includes a polypeptide having at least 80% sequence identity with the amino acid sequence of SEQ ID NO: 27.

The length of the spacer is, for example, 1 to 10 amino acid residues (for example, 2 to 4 amino acid residues). An example of the spacer includes consecution of glycine and serine (such as GCG, GCGC, or GCGCG).

A present polypeptide may contain a signal peptide for expressing/localizing a present polypeptide on the cell membrane of an immune cell. The signal peptide is not especially limited as long as it does not inhibit the function of the present polypeptide, and enables the present polypeptide to express/localize on the cell membrane of an immune cell. The length of the signal peptide is, for example, 10 to 70 amino acid residues, preferably 15 to 60 amino acid residues, and more preferably 15 to 30 amino acid residues. The linking site of the signal peptide is not especially limited, and may be the N terminal or the carboxyl (C) terminal of the present polypeptide, between a present cytokine extracellular domain and a transmembrane domain, between a transmembrane domain and a present TNFRSF molecule intracellular domain, between a present cytokine extracellular domain and a first cell membrane binding domain, or between a present TNFRSF molecule intracellular domain and a second cell membrane binding domain, and is preferably the N terminal of the present polypeptide (namely, a present membrane-bound cytokine-TNFRSF molecule chimeric ligand, a present first polypeptide, or a present second polypeptide). An example of the signal peptide includes a signal peptide derived from a naturally present secretory protein (such as the cytokine), or a membrane protein (such as the co-stimulatory molecule), and a CD8α-derived signal peptide and an IL2-derived signal peptide are preferred. A specific example of the CD8α-derived signal peptide includes a polypeptide having at least 80% sequence identity with the amino acid sequence of SEQ ID NO: 4, and a specific example of the IL2-derived signal peptide includes a polypeptide having at least 80% sequence identity with the amino acid sequence of SEQ ID NO: 3.

A present polypeptide is designed so that a polynucleotide encoding the present polypeptide (namely, a present polynucleotide) can be expressed in an immune cell, and hence is usually expressed in an immune cell. Here, the immune cell is not especially limited as long as it is a cell that performs an immune function in a living body, and a preferable example includes an immune cell selected from a T cell and an NK cell because the effect has been verified in Examples described below. An immune cell to which the present polynucleotide is to be genetically introduced, and an immune cell in which the present polypeptide is to be expressed may be an immune cell present in a living body (in vivo), and is usually an immune cell present outside a living body.

Regarding a present cytokine extracellular domain encoding region contained in the present polynucleotide, those skilled in the art can specifically and definitely grasp a nucleotide sequence corresponding the amino acid sequence by referring to the amino acid sequence of the present cytokine extracellular domain, and known codon tables corresponding to various species. For example, when the present cytokine extracellular domain is a polypeptide containing an amino acid sequence of an extracellular domain derived from human IL15 (amino acid sequence having at least 80% sequence identity with the amino acid sequence of SEQ ID NO: 22), an example includes a cDNA (polynucleotide) encoding an extracellular domain derived from human IL15 (namely, a polynucleotide containing a nucleotide sequence having at least 80% sequence identity with the nucleotide sequence of SEQ ID NO: 49).

Regarding a present TNFRSF molecule intracellular domain encoding region contained in the present polynucleotide, those skilled in the art can specifically and definitely obtain a nucleotide sequence corresponding to the amino acid sequence by referring to the amino acid sequence of a region derived from a TNFRSF molecule intracellular domain, and known codon tables corresponding to various species. For example, when the region derived from a TNFRSF molecule intracellular domain is a polypeptide containing an amino acid sequence derived from an intracellular domain of human TNFR2 (amino acid sequence having at least 80% sequence identity with the amino acid sequence of SEQ ID NO: 5), an example includes a cDNA (polynucleotide) encoding a region derived from an intracellular domain of human TNFR2 (namely, a polynucleotide containing a nucleotide sequence having at least 80% sequence identity with the nucleotide sequence of SEQ ID NO: 50).

The present polynucleotide may contain a region (polynucleotide) encoding a signal peptide for expressing/localizing a present polypeptide on the cell membrane of an immune cell.

The present polynucleotide may contain a polynucleotide encoding a linker peptide and/or a polynucleotide encoding a spacer in one or more sites selected from the upstream of the present polynucleotide; the downstream of the present polynucleotide; between a present cytokine extracellular domain encoding region and a transmembrane domain encoding region; between a transmembrane domain encoding region and a present TNFRSF molecule intracellular domain encoding region; between a present cytokine extracellular domain encoding region and a first cell membrane binding domain encoding region; and between a second cell membrane binding domain encoding region and a present TNFRSF molecule intracellular domain encoding region.

Herein, “upstream” means, assuming that the present polynucleotide is operably linked downstream of a promoter of the present vector, the end of the present polynucleotide on a side closer to the promoter (the upstream side in the transcription direction from the promoter), and “downstream” means the end of the present polynucleotide on a side farther from the promoter (the downstream side in the transcription direction from the promoter).

Herein, “polynucleotide designed to express in an immune cell” more specifically means a polynucleotide designed so that when a vector containing a promoter functioning in an immune cell, and a polynucleotide operably linked downstream of the promoter is introduced into an immune cell, a target polypeptide encoded by the polynucleotide (namely, a present membrane-bound cytokine-TNFRSF molecule chimeric ligand or a present polypeptide combination) can express in the immune cell.

When an immune cell to which the present polynucleotide is to be genetically introduced is not an immune cell expressing a peptide/polypeptide for treatment or prevention of a diseases, and it is necessary to prepare an immune cell for treatment or prevention for an arbitrary disease by using the present polynucleotide, the present polynucleotide preferably further contains a polynucleotide encoding at least one peptide/polypeptide for such treatment or prevention.

The present polynucleotide may be in the form of an mRNA, a cDNA or the like containing a present membrane-bound cytokine-TNFRSF molecule chimeric ligand encoding region or a present polypeptide combination encoding region, or in the form of a plasmid, a vector, a virus, or the like suitable for gene introduction for expressing the present polypeptide on the cell membrane.

A nucleotide contained in the present polynucleotide may be a DNA or an RNA having a natural structure, or may be a nucleotide having a structure obtained by chemically modifying a DNA or an RNA having a natural structure (also referred to as a “modified nucleic acid”). For example, when the present polynucleotide is provided to a cell in the form of an mRNA, a modified nucleic acid is used for imparting resistance to decomposition by RNase. The modified nucleic acid is preferably modified in a base moiety of the nucleotide, and may be, for example, a pyrimidine nucleotide substituted at the 5-position, or pseudouridine optionally substituted at the 1-position, and specific examples include 5-methylcytidine, 5-methoxyuridine, 5-methyluridine, pseudouridine, and 1-alkylpseudouridine. Besides, 1-alkylpsudouridine may be 1-(C1-C6 alkyl)pseudouridine, and is preferably 1-methylpseudouridine or 1-ethylpseudouridine.

The present polynucleotide can be easily produced by a usual method based on the amino acid sequence of the present polypeptide. A nucleotide sequence encoding the amino acid sequence can be obtained based on the amino acid sequence described in Sequence Listing, and the present polynucleotide can be produced by employing standard molecular biological and/or chemical procedures. For example, a polynucleotide can be synthesized based on the nucleotide sequence, and the present polynucleotide can be produced by combining DNA fragments obtained from a cDNA library by polymerase chain reaction (PCR).

The present polynucleotide may contain a nucleotide sequence having a codon optimized for expression in a specific host cell. Such an optimized nucleotide sequence can be obtained by applying a known algorithm or software to a target amino acid sequence.

The present polynucleotide may be a single-stranded (sense chain) polynucleotide encoding the present polynucleotide, or may be a double-stranded polynucleotide consisting of the sense chain and an antisense chain having a complementary sequence, and the form is selected to be suitable for a method for introducing the polynucleotide into a cell. For example, when an mRNA or a lentiviral vector is used, it is in a single-stranded form, and when a plasmid DNA is used, it can be in a double-stranded form.

### <2-1. Present Membrane-Bound Cytokine-TNFRSF Molecule Chimeric Ligand>

One aspect of the present polypeptide is a membrane-bound cytokine-TNFRSF molecule chimeric ligand containing a present cytokine extracellular domain and a present TNFRSF molecule intracellular domain directly connected to each other, and specifically a chimeric polypeptide containing: an extracellular domain containing an amino acid sequence derived from a ligand protein that binds to a receptor of IL15, IL2, or IL7, wherein the binding of the ligand protein to the receptor transmits a signal similar to a binding signal of the cytokine into an immune cell via the receptor; a transmembrane domain; and an intracellular domain containing a region derived from an intracellular domain of a TNFRSF molecule, but not containing an ITAM intracellular signaling region.

The present membrane-bound cytokine-TNFRSF molecule chimeric ligand may be any chimeric ligand as long as the present cytokine extracellular domain and the present TNFRSF molecule intracellular domain are linked to each other via a transmembrane domain on a single polypeptide when the present membrane-bound cytokine-TNFRSF molecule chimeric ligand is expressed in an immune cell. As long as the present cytokine extracellular domain is present outside the cell (where an endogenous cytokine is originally present), and the present TNFRSF molecule intracellular domain is present inside the cell (where an endogenous TNFRSF molecule intracellular domain is originally present), the present cytokine extracellular domain, the transmembrane domain, and the present TNFRSF molecule intracellular domain may be linked in the stated order from the amino (N) terminal, or the present TNFRSF molecule intracellular domain, the transmembrane domain, and the present cytokine extracellular domain may be linked in the stated order from the N terminal.

The membrane-bound cytokine-TNFRSF molecule chimeric ligand is preferably a chimeric ligand of a present membrane-bound cytokine-TNFRSF molecule using IL15 or IL7 as a present cytokine, more preferably a chimeric ligand of a present membrane-bound cytokine-TNFRSF molecule using IL15 or IL7 as the present cytokine, and using OX40 or CD17 as a TNFSF molecule, and further preferably a chimeric ligand, using a CD8α linker and transmembrane domain, of a present membrane-bound cytokine-TNFRSF molecule of IL15TM-OX40 (amino acids 21 to 257 of SEQ ID NO: 52), IL15TM-CD137 (amino acids 21 to 262 of SEQ ID NO: 53), IL7TM-OX40 (amino acids 21 to 295 of SEQ ID NO: 54), or IL7TM-CD137 (amino acids 21 to 300 of SEQ ID NO: 55).

In a present membrane-bound cytokine-TNFRSF molecule chimeric ligand encoding region, as long as a present cytokine extracellular domain of a present cytokine-TNFRSF molecule chimeric ligand is present outside the cell (where an endogenous cytokine is originally present), and a present TNFRSF molecule intracellular domain of the present membrane-bound cytokine-TNFRSF molecule chimeric ligand is present inside the cell (where an endogenous TNFRSF molecule intracellular domain is originally present) when the present membrane-bound cytokine-TNFRSF molecule chimeric ligand is expressed in an immune cell, a present cytokine extracellular domain encoding region, a transmembrane domain encoding region, and a present TNFRSF molecule intracellular domain encoding region may be linked in the stated order from the upstream, or a present TNFRSF molecule intracellular domain encoding region, a transmembrane domain encoding region, and a present cytokine extracellular domain encoding region may be linked in the stated order from the upstream.

Regarding a transmembrane domain encoding region contained in the present membrane-bound cytokine-TNFRSF molecule chimeric ligand encoding region, those skilled in the art can specifically and definitely obtain a nucleotide sequence corresponding to the amino acid sequence by referring to the amino acid sequence of a transmembrane domain, and known codon tables corresponding to various species. For example, when the transmembrane domain is a polypeptide containing an amino acid sequence of human CD8α-derived transmembrane domain (amino acid sequence having at least 80% sequence identity with the amino acid sequence of SEQ ID NO: 16), an example includes a cDNA (polynucleotide) encoding the human CD8α-derived transmembrane domain (namely, a polynucleotide containing a nucleotide sequence having at least 80% sequence identity with the nucleotide sequence of SEQ ID NO: 51).

### <2-2 Present Polypeptide Combination>

One aspect of the present polypeptide is a polypeptide combination of a first polypeptide containing a present cytokine extracellular domain and a second polypeptide containing a present TNFRSF molecule intracellular domain which are expressed in combination on the same immune cell, and more specifically, a polypeptide combination of: a first polypeptide containing a present cytokine extracellular domain and a first cell membrane binding domain (namely, a present first polypeptide); and a second polypeptide containing a second cell membrane binding domain and a present TNFRSF molecule intracellular domain (namely, a present second polypeptide).

The present first polypeptide used in the present polypeptide combination is a chimeric polypeptide substantially containing a present cytokine extracellular domain, and a first cell membrane binding domain. The intracellular domain thereof does not contain a signaling domain, but may contain a linker peptide, a spacer, or the like. In the present first polypeptide, as long as the present cytokine extracellular domain is present outside the cell (where an endogenous cytokine is originally present) when the present first polypeptide is expressed in an immune cell, the present cytokine extracellular domain and the first cell membrane binding domain may be linked in the stated order from the N terminal, or the first cell membrane binding domain and the present cytokine extracellular domain may be linked in the stated order from the N terminal. Besides, the present first polypeptide may contain, as the intracellular domain thereof, an amino acid sequence having substantially no biological activity. Examples of such an amino acid sequence include an amino acid sequence used as a spacer, a linker peptide, and a hinge peptide.

The present second polypeptide used in the present polypeptide combination is a chimeric polypeptide containing a second cell membrane binding domain and an endogenous TNFRSF molecule intracellular domain, and containing, as the extracellular region thereof, a linker peptide, and a structure optionally including an amino acid sequence or the like corresponding to a present membrane-bound ligand described below. Besides, the present second polypeptide is different from a CAR molecule and a CAAR molecule in that an ITAM intracellular signaling region is not contained in the intracellular domain. In the present second polypeptide used in the present polypeptide combination, as long as the present TNFRSF molecule intracellular domain is present inside the cell (where an endogenous TNFRSF molecule intracellular domain is originally present) when the present second polypeptide is expressed in an immune cell, the present TNFRSF molecule intracellular domain and the cell membrane binding domain may be linked in the stated order from the N terminal, or the cell membrane binding domain and the present TNFRSF molecule intracellular domain may be linked in the stated order from the N terminal.

“(First or second) cell membrane binding domain” used in the present polypeptide combination means a polypeptide having a property of binding to the cell membrane of an immune cell. Examples of the (first or second) cell membrane binding domain include a transmembrane domain, a lipid anchor, and a glycolipid anchor. When the first cell membrane binding domain of the present first polypeptide, and the second cell membrane binding domain of the present second polypeptide are transmembrane domains, the present first polypeptide and the present second polypeptide can be designated as endogenous membrane polypeptides. When the cell membrane binding domains of the present first polypeptide and the present second polypeptide are lipid anchors or glycolipid anchors, the present first polypeptide and the present second polypeptide can be designated as superficial membrane polypeptides. The first cell membrane binding domain and the second cell membrane binding domain are preferably transmembrane domains, and since the effect has been verified in Examples described below, it is more preferable to use a CD8α-derived transmembrane domain, or a CD28-derived transmembrane domain as the first cell membrane binding domain, and to use a CD8α-derived transmembrane domain, or a CD28-derived transmembrane domain as the second cell membrane binding domain.

The second cell membrane binding domain of the present second polypeptide may be 1) a cell membrane binding domain derived from the same TNFRSF molecule as the TNFRSF molecule of the present second polypeptide, or 2) a cell membrane binding domain derived from a TNFRSF molecule different from the TNFRSF molecule of the present second polypeptide, or a cell membrane binding domain derived from a polypeptide except for a TNFRSF molecule. In the case of 1), the TNFRSF molecule itself can be used as the present second polypeptide, but it is preferable that an extracellular domain of the TNFRSF molecule (a region in the TNFRSF molecule that binds to a ligand) is not contained. In the case of 2), the intracellular domain of the TNFRSF molecule and the second cell membrane binding domain are in a chimeric relationship.

Since the present second polypeptide is different from a CAR, it is preferable that the present second polypeptide does not contain a binding domain for a target molecule (for example, a single chain antibody for a target molecule) present in a cell targeted by an immune cell in which the present second polypeptide is to be expressed.

The present second polypeptide is preferably a polypeptide containing an extracellular domain containing an amino acid sequence derived from a ligand molecule of a receptor present in an immune cell in which the present polypeptide combination is to be expressed (namely, a molecule of type (IV) described in Examples below [present membrane-bound ligand molecule-TNFRSF molecule chimeric ligand]).

Examples of the ligand molecule of the present membrane-bound ligand molecule-TNFRSF molecule chimeric ligand include a cytokine, a chemokine, Wnt, and TGFβ. Examples of the cytokine include IL2, IL4, IL6, IL7, IL9, IL15, IL21, IL1α, IL1β, IL18, IL33, IL36, IL37, and IL38, and since the effect has been verified in Examples described below, a preferable example includes a cytokine selected from IL7 and IL21. The ligand molecule of the present membrane-bound ligand molecule-TNFRSF molecule chimeric ligand is preferably a present cytokine except for a membrane-bound cytokine used in a present first polypeptide. Specifically, when IL2TM or IL7TM is used as the membrane-bound cytokine of the present first polypeptide, the ligand molecule of the present membrane-bound ligand molecule-TNFRSF molecule chimeric ligand is preferably IL15.

Examples of the extracellular domain containing an amino acid derived from the ligand molecule of the present membrane-bound ligand molecule-TNFRSF molecule chimeric ligand include, in addition to an amino acid sequence of an extracellular domain derived from IL15, IL2, or IL7 of human, mouse, or rat described above:
a polypeptide containing an amino acid sequence having at least 80% sequence identity with the amino acid sequence of human IL4 (SEQ ID NO: 18), and retaining activity of binding to a human IL4 receptor;
a polypeptide containing an amino acid sequence having at least 80% sequence identity with the amino acid sequence of human IL6 (SEQ ID NO: 19), and retaining activity of binding to a human IL6 receptor;
a polypeptide containing an amino acid sequence having at least 80% sequence identity with the amino acid sequence of human IL9 (SEQ ID NO: 21), and retaining activity of binding to a human IL9 receptor; and
a polypeptide containing an amino acid sequence having at least 80% sequence identity with the amino acid sequence of human IL21 (SEQ ID NO: 23), and retaining activity of binding to a human IL21 receptor.

In the present first polypeptide encoding region used in the present polypeptide combination encoding region, as long as the present cytokine extracellular domain is present outside the cell (where an endogenous cytokine is originally present) when the present first polypeptide encoding region is expressed in an immune cell, the present cytokine extracellular domain encoding region and the first cell membrane binding domain encoding region may be linked in the stated order from the upstream, or the first cell membrane binding domain encoding region and the present cytokine extracellular domain encoding region may be linked in the stated order from the upstream. In the present second polypeptide encoding region used in the present polypeptide combination encoding region, as long as the present TNFRSF molecule intracellular domain is present inside the cell (where an endogenous TNFRSF molecule intracellular domain is originally present) when the present second polypeptide encoding region is expressed in an immune cell, the present TNFRSF molecule intracellular domain encoding region and the second cell membrane binding domain encoding region may be linked in the stated order from the upstream, or the second cell membrane binding domain encoding region and the present TNFRSF molecule intracellular domain encoding region may be linked in the stated order from the upstream.

Regarding a (first or second) transmembrane domain encoding region used in the present polypeptide combination encoding region, those skilled in the art can specifically and definitely obtain a nucleotide sequence corresponding to the amino acid sequence by referring to the amino acid sequence of a (first or second) cell membrane binding domain, and known codon tables corresponding to various species. For example, when the (first or second) cell membrane binding domain is a polypeptide containing an amino acid sequence of human CD8α-derived transmembrane domain (amino acid sequence having at least 80% sequence identity with the amino acid sequence of SEQ ID NO: 16), an example includes a cDNA (polynucleotide) encoding the human CD8α-derived transmembrane domain (namely, a polynucleotide containing a nucleotide sequence having at least 80% sequence identity with the nucleotide sequence of SEQ ID NO: 51).

The present first polypeptide encoding region and the present second polypeptide encoding region of the present polypeptide combination encoding region may be located in a combination of separate polynucleotides, or a single polynucleotide. When these encoding regions are located in a single polynucleotide, the present first polypeptide and the present second polypeptide can be independently expressed in the immune cell by linking the present first polypeptide encoding region and the present second polypeptide encoding region via a polynucleotide encoding a self-cleaving peptide (also designated as a self-cutting peptide). Herein, examples of the self-cleaving peptide include a 2A peptide derived from foot-and-mouth disease virus (FMDV) (self-cleaving peptide F2A), a 2A peptide derived from equine rhinitis A virus (ERAV) (self-cleaving peptide E2A), a 2A peptide derived from porcine teschovirus-1 (PTV-1) (self-cleaving peptide P2A), and a 2A peptide derived from Thoseaasigna virus (TaV) (self-cleaving peptide T2A), and the self-cleaving peptide T2A is preferred. A specific example of the self-cleaving peptide T2A includes a polypeptide having at least 80% sequence identity with the amino acid sequence of SEQ ID NO: 14.

Since the present second polypeptide encoding region of the present polypeptide combination encoding region is different from a polynucleotide encoding a CAR, the encoding region preferably does not contain a polynucleotide encoding a binding domain for a target molecule (for example, a single chain antibody for a target molecule) present in a cell targeted by the immune cell to which the present polypeptide combination encoding region is to be genetically introduced.

Since the effect has been verified in Examples described below, the present second polypeptide encoding region of the present polypeptide combination encoding region is preferably a polynucleotide encoding an extracellular domain containing an amino acid sequence derived from a ligand molecule of a receptor present in the immune cell to which the present polypeptide combination encoding region is to be genetically introduced (namely, a polynucleotide encoding a present membrane-bound ligand molecule-TNFRSF molecule chimeric ligand).

### 3. Polypeptide Expression Technology and Present Immune Cell

The present invention provides a method for genetically introducing a vector containing a present polynucleotide (namely, a present vector) to an immune cell, an immune cell to which the present vector has been introduced (namely, a present immune cell (1)), an immune cell in which a present polypeptide is expressed (namely, a present immune cell (2-1) or a present immune cell (2-2)), a method for producing (preparing) a present immune cell, and the like.

The present vector is not especially limited as long as it contains a promoter, and a present polynucleotide operably linked downstream of the promoter, and can transcribe an mRNA encoded by the present polynucleotide.

The present vector containing a present polypeptide combination encoding region can be a vector that contains a first promoter, and a present first polypeptide encoding region operably linked downstream of the first promoter, and further contains a second promoter, and a present second polypeptide encoding region operably linked downstream of the second promoter, or a combination of a vector containing a first promoter, and a present first polypeptide encoding region operably linked downstream of the first promoter, and a vector containing a second promoter, and a present second polypeptide encoding region operably linked downstream of the second promoter, or alternatively, may be a vector in which a present first polypeptide encoding region and a present second polypeptide encoding region are operably linked downstream of one promoter. In the latter vector, when the present first polypeptide encoding region and the present second polypeptide encoding region are linked via a polynucleotide encoding a self-cleaving peptide, a present first polypeptide and a present second polypeptide can be independently expressed in an immune cell.

The present vector can be appropriately selected in accordance with the purpose, and examples include a non-viral vector (such as an episomal vector, an artificial chromosome vector, or a plasmid vector), and a viral vector. Besides, the vector may be cyclic, or linear.

The promoter used in the present vector is not especially limited as long as an RNA polymerase (preferably an RNA polymerase, and a general transcription factor) binds thereto, and it is a region for starting transcription of an mRNA encoded by a present polynucleotide positioned downstream thereof. Examples include an SRU promoter, a SV40 initial promoter, an LTR (Long Terminal Repeat) of a virus, a CMV (cytomegalovirus) promoter, an RSV (Rous sarcoma virus) promoter, an HSV-TK (herpes simplex virus-thymidine kinase) promoter, an EF1α promoter, a metallothionein promoter, and a heat shock promoter. Alternatively, an enhancer of IE gene of human CMV may be used together with the promoter. For example, a CAG promoter (containing a cytomegalovirus enhancer, a chicken β-actin promoter, and a poly A signal site of β-globin gene) can be used.

The episomal vector is a vector autonomously replicable outside the chromosome. A specific method of using an episomal vector is disclosed in Yu et al., Science, 324, 797-801 (2009). In one embodiment of the present invention, an episomal vector in which loxP sequences are disposed in the same direction on the 5′ end side and 3′ end side of a vector element necessary for replication of the episomal vector can be used. Since an episomal vector can be autonomously replicated outside the chromosome, it can stably express in a host cell without being incorporated into the genome.

An example of the episomal vector includes a vector containing, as a vector element, a sequence necessary for autonomous replication, and derived from EBV, SV40, or the like. The vector element necessary for autonomous replication can be specifically a gene encoding a replication origin, and a protein for controlling replication by binding to the replication origin, and can be replication origin oriP, and EBNA-1 gene in using EBV, and replication origin ori, and SV40LT gene in using SV40.

Examples of the artificial chromosome vector include a YAC (Yeast Artificial Chromosome) vector, a BAC (Bacterial Artificial Chromosome) vector, and a PAC (P1-derived Artificial Chromosome) vector.

Examples of the plasmid vector include pA1-11, pXT1, pRc/CMV, pRc/RSV, and pcDNAI/Neo.

The viral vector means a gene vector utilizing infectivity or replicability of a virus, and more specifically means a virion containing a viral vector plasmid (that can be a DNA or an RNA) obtained by removing a gene involved in pathogenicity from a virus genome, and incorporating an exogenous gene (a present polynucleotide in the present application) thereinto (which vector is also designated as a “recombinant virus”). Examples of the viral vector include a retroviral vector, a lentiviral vector, an adenoviral vector, an adeno-associated viral (AAV) vector, a Sendai virus vector, a herpes virus vector, a vaccinia virus vector, a poxvirus vector, a polio virus vector, a silvis virus vector, a rhabdovirus vector, a paramyxovirus vector, and an orthomyxovirus vector.

The viral vector (namely, the recombinant virus) can be obtained by transfecting a plasmid vector for virus production containing a present polynucleotide, a region necessary for virus occlusion (such as an LTR), and the like; or such a plasmid vector for virus production, and a packaging plasmid expressing constitutive genes necessary for forming a virion (such as gag, pol, and env) into a packaging cell, such as PG13 cell line (ATCC [American Type Culture Collection], CRL-10686), PA317 cell line (ATCC, CRL-9078), Lenti-X293 T cell line (manufactured by Takara Bio Inc., 632180), PLAT-A cell line, PLAT-E cell line, PLAT-F cell line, or PLAT-GP cell line, by a method such as a lipofection method, an electroporation method, or a calcium phosphate method, collecting a culture supernatant containing a recombinant virus, and concentrating the recombinant virus if necessary. For the concentration of the recombinant virus, an appropriate kit in accordance with the type of virus (such as Lenti-X Concentrator [manufactured by Takara Bio Inc., 631232]) can be used. Examples of the plasmid vector for virus production include pLVSIN EF1α pur (manufactured by Takara Bio Inc., 6186) and pLVSIN-IRES-ZsGreen1 (manufactured by Takara Bio Inc., 6191) that are plasmid vectors for lentivirus production, pQCXIX (manufactured by Takara Bio Inc., Z1515N) that is a plasmid vector for retrovirus production, and pAAV-CMV (manufactured by Takara Bio Inc., 6651) that is a plasmid vector for AAV production. When the plasmid vector for virus production, or the packaging plasmid is to be transfected into a cell, an introduction aid reagent (such as Opti-MEM IReduced Serum Media [manufactured by Thermo Fisher Scientific, 31985070]) may be used. The transfection of the plasmid vector for virus production, and the packaging plasmid can be performed by using a kit appropriately selected in accordance with the type of virus, and for example, when the virus vector is a lentivirus, for example, Lentiviral High Titer Packaging Mix (manufactured by Takara Bio Inc., 6194) or the like can be used. Besides, as the packaging cell, for example, 293 cell, or 293 T cell having high transfection efficiency (specifically, Lenti-X293 T cell line described above) can be used.

A specific example of a method for producing a lentivirus includes a method in which the plasmid for virus production, Lentiviral High Titer Packaging Mix described above, and TransIT-293 Transfection Reagent (manufactured by Takara Bio Inc., MIR2704) are mixed in Opti-MEM I Reduced Serum Media described above, and incubated for 15 minutes, the resultant is then added to Lenti-X 293 T cell line described above having been precedently cultured to semi-confluent, the resultant mixture is cultured for 24 to 48 hours, and then, a lentivirus contained in the culture fluid is collected, and if necessary, the lentivirus is concentrated with Lenti-Xconcentrator (Takara, 631232) in accordance with a protocol.

The present vector may contain, in addition to the promoter, an enhancer if necessary, a poly A addition signal, a marker gene, a replication origin, a gene encoding a polypeptide bound to the replication origin to control replication, and the like. The marker gene refers to a gene with which cells can be sorted or selected through gene introduction of the marker gene into the cells. Specific examples of the marker gene include a drug resistance gene, a fluorescent protein gene, a luminescent enzyme gene, and a chromogenic enzyme gene. One of these may be singly used, or two or more of these may be used together. Specific examples of the drug resistance gene include a neomycin resistance gene, a tetracycline resistance gene, a kanamycin resistance gene, a Zeocin resistance gene, and a hygromycin resistance gene. Specific examples of the fluorescent protein gene includes a blue fluorescent protein (BFP) gene, a green fluorescent protein (GFP) gene, a yellow fluorescent protein (YFP) gene, and a red fluorescent protein (RFP) gene. A specific example of the luminescent enzyme gene includes a luciferase gene. Specific examples of the chromogenic enzyme gene include a β galactosidase gene, a β glucuronidase gene, and an alkali phosphatase gene.

The present immune cell (1) may be any immune cell in which a present vector has been introduced, and a present polypeptide (namely, a present membrane-bound cytokine-TNFRSF molecule chimeric ligand, or a present polypeptide combination) is to be expressed on the cell membrane. The present immune cell (2-1) may be any immune cell in which a present membrane-bound cytokine-TNFRSF molecule chimeric ligand is to be expressed on the cell membrane. The present immune cell (2-2) may be any immune cell in which a present polypeptide combination is to be expressed on the cell membrane. A present immune cell survives/is maintained usually in a liquid, or in a moistened (wet) state with a liquid in a container (such as a culture plate or dish, or a tube for storing or sorting cells). Such a liquid is not especially limited as long as the present immune cell can survive or be maintained therein, and examples include liquids such as a culture fluid (such as a serum-containing or -free, and/or a culture fluid containing or not containing the cytokine), saline, phosphate buffered saline, tris-buffered saline, HEPES buffered saline, Ringer's solution (such as lactated Ringer's solution, acetated Ringer's solution, or bicarbonated Ringer's solution), and 5% aqueous solution of glucose. An example of the serum includes 0.1 to 30 (v/v) % serum (such as fetal bovine serum [FBS], or calf bovine serum [CS]). An example of the culture fluid includes a culture fluid for culturing animal cells (such as DMEM, EMEM, IMDM, PRMI1640, αMEM, F-12, F-10, M-199, or AIM-V). An example of the serum-free culture fluid includes a culture fluid for culturing animal cells supplemented with an appropriate amount (for example, 1 to 30%) of commercially available B27 supplement (-insulin) (manufactured by Life Technologies), N2 supplement (manufactured by Life Technologies), B27 supplement (manufactured by Life Technologies), or a serum replacement such as Knockout Serum Replacement (manufactured by Invitrogen). Since the present immune cell can survive/be maintained under a cytokine-free condition, the culture fluid is preferably a cytokine-free culture fluid.

A present membrane-bound cytokine-TNFRSF molecule chimeric ligand expressed in a present immune cell (1) and a present immune cell (2-1) is not endogenous in the immune cell, and hence is inevitably exogenous. A present first polypeptide expressed in a present immune cell contains a present cytokine extracellular domain, and a (first) cell membrane binding domain, and is not endogenous in the immune cell, and hence is inevitably exogenous. On the other hand, although a present second polypeptide expressed in a present immune cell encompasses, in some aspects, a polypeptide the same as that endogenous in the immune cell, it is not endogenous but exogenous.

A present immune cell can be produced by genetically introducing a present vector to an immune cell. A method for genetically introducing the present vector to an immune cell may be any method suitable to the present vector and the immune cell. For example, when a non-viral vector is used as the present vector, examples of the method include a method using a microparticle containing liposome, cationic lipid, or the like as described in, for example, International Publication Nos. WO96/10038, WO97/18185, WO97/25329, WO97/30170, and WO97/31934 (all of which are incorporated herein by reference); a method in which an episomal vector expressing a scaffold/matrix attachment region element is introduced as described in the literature “Jin et al., EMBO Mol Med. 2016 July; 8(7): 702-711.”; a method using a shell having the content destroyed by radiation exposure of a virion as employed in a transposon vector introduction method such as PiggyBac method described in the literature “Mol Ther Methods Clin Dev. 2017 Dec. 22; 8: 131-140.”; and a method in which a present vector (specifically, a linear present vector) is introduced into a genome of a target cell by genome editing technology using, for example, CRISPR/Cas9, or Zn finger nuclease (for example, U.S. Pat. No. 8,956,828).

Alternatively, when a viral vector (namely, a recombinant virus) is used as the present vector, an example of a method for genetically introducing the present vector to an immune cell includes a method in which the immune cell is infected with the virus by using a culture supernatant containing the recombinant virus described above, or the recombinant virus having been concentrated.

A present immune cell (2-1) and a present immune cell (2-2) can be produced also by introducing a present polypeptide into an immune cell. A method for genetically introducing the present polypeptide to an immune cell is not especially limited, and any of known methods can be appropriately selected to be employed. Examples of such a method include a method using a protein transduction reagent, a method using a protein transduction domain (PTD) fused protein, and a micro-injection method. As the protein transduction reagent, BioPORTER® Protein Delivery Reagent (manufactured by Gene Therapy Systems), and Pro-Ject™ Protein Transfection Reagent (manufactured by PIERCE) both based on a cationic lipid, Profect-1 (manufactured by Targeting Systems) based on a lipid, Penetratin Peptide (manufactured by Qbiogene), and Chariot Kit (manufactured by Active Motif) both based on membrane-permeable peptide, and GenomONE (manufactured by Ishibara Sangyo Kaisha Ltd.) using HVJ envelope (inactivated Sendai virus) are commercially available.

A present immune cell (2-2) is preferably an immune cell in which a vector carrying a first polypeptide and a second polypeptide has been introduced; or an immune cell in which a vector carrying a first polypeptide, and a vector carrying a second polypeptide have been introduced. The “vector carrying a first polypeptide and a second polypeptide” may be any vector as long as the first polypeptide and the second polypeptide are carried on the same vector. Specific examples include, as described above, a vector containing a first promoter, and a present first polypeptide encoding region operably linked downstream of the first promoter, and further containing a second promoter, and a present second polypeptide encoding region operably linked downstream of the second promoter, and a vector in which a present first polypeptide encoding region and a present second polypeptide encoding region are operably linked downstream of one promoter, and the present first polypeptide encoding region and the present second polypeptide encoding region are linked via a polynucleotide encoding a self-cleaving peptide. Besides, the “vector carrying a first polypeptide, and the vector carrying a second polypeptide” may be any different vectors respectively carrying the first polypeptide and the second polypeptide. Here, an example of the “vector carrying a first polypeptide” specifically includes a vector containing a first promoter, and a present first polypeptide encoding region operably linked downstream of the first promoter described above. An example of the “vector carrying a second polypeptide” specifically includes a vector containing a second promoter, and a present second polypeptide encoding region operably linked downstream of the second promoter described above.

The immune cell can be obtained by isolating from and purifying a body fluid such as blood or bone marrow aspirate, a tissue of the spleen, the thymus, or the lymph node, or an immune cell infiltrating into a cancer tissue of primary tumor, metastatic tumor, or cancerous ascites. Besides, when the immune cell is a T cell, it can be obtained by isolating a peripheral blood mononuclear cell (PBMC), and subjecting the resultant to anti-CD3 antibody stimulation.

When the present immune cell is applied to autologous cell therapy, the present treatment or prevention method may further include, before the step of administering the present immune cell to a subject requiring treatment or prevention of the disease, the following steps of:
a) collecting an immune cell from the subject requiring treatment or prevention of the disease;
b) genetically introducing a vector containing the present polynucleotide (namely, the present vector) to the collected immune cell; and
c) culturing (expansion-culturing) a cell population including the present immune cell.

In one aspect, the collected immune cell (preferably, a cell population containing a T cell or an NK cell, more preferably a PBMC or CD3-positive cell fraction) may be activated, before the gene introduction, by treatment with a stimulating factor in accordance with the type and property of the immune cell. When the immune cell is a T cell, it is usually stimulated with a soluble or membrane bound anti-CD3 antibody (such as OKT3, or mOKT3), and/or an antigen presenting cell (such as artificial antigen present cell [aAPC]; or antigen presenting cell expressing a membrane anti-CD3 monoclonal antibody), and another suitable stimulating factor/condition may be appropriately selected in accordance with the type and property of the T cell. Alternatively, when the immune cell is an NK cell, an anti-CD16 antibody, IL2, IL18, or the like is used as the stimulating factor. Besides, when a mixture of a T cell and an NK cell, such as PBMC, is used, an appropriate combination of the above-described stimulating factors may be used.

In another aspect, an immune cell to which a present vector is to be genetically introduced is suspended in a prescribed cell concentration (of, for example, 0.1 to 2×10⁶ cells/mL) to be added to a virus binding plate. Such a virus binding plate can be produced by adding the recombinant virus concentrate described above to a plate coated with 5 to 10 μg/mL anti-CD3 antibody (clone name: OKT3) and 20 to 100 μg/mL RetroNectin.

As the culture fluid used in the gene introduction, for example, the serum or serum replacement described above, or a culture fluid supplemented with a cytokine such as IL-2 (for example, AIM-V [manufactured by Thermo Fisher Scientific, 12055083]) can be used. The virus binding plate to which the immune cell has been added may be centrifuged.

The step b) may be repeated a plurality of times (for example, twice to 10 times, preferably 2, 3, 4, or 5 times) so as to attain a sufficient level of expression of the present polypeptide in the immune cell.

In one aspect, the step b) may be performed continuously for 2 days or more, for example, continuously for 2 days, continuously for 3 days, or continuously for 4 days. Besides, when the present vector is genetically introduced to the immune cell using a virus binding plate, the culture may be performed continuously for 5 to 20 days with the culture fluid exchanged every 2 or 3 days.

When a present immune cell is applied to allogenic cell therapy, a vector containing a present polynucleotide (namely, a present vector) can be introduced into a stem cell (for example, a pluripotent stem cell such as an ES cell, or an iPS cell; or a cell differentiation induced from a pluripotent stem cell to a blood cell) by employing a usual gene introduction method, a gene editing method using CRISPR/CAS9 or the like. For example, in using such a pluripotent stem cell, a present vector is introduced into the genome of the pluripotent stem cell by genome editing technology, so as to produce an appropriately genetically introduced pluripotent stem cell. Since a pluripotent stem cell can be amplified, when a necessary amount of the cell is amplified at a necessary timing to cause differentiation induction to an immune cell such as a T cell, this method can be applied to allogenic therapy.

### 4. Present medicament, and Method for Treating or Preventing Disease

The present invention provides a medicament containing a present immune cell, and a method for treating or preventing a disease by administering the present immune cell.

A present pharmaceutical may consist of a present immune cell itself, or may be in the form of a composition (namely, a pharmaceutical composition) containing a present immune cell and an additive. Examples of the additive include pharmaceutically acceptable usual components such as a carrier, a binder, a stabilizer, an excipient, a diluent, a pH buffer, an isotonic agent, a coating agent, a solubilizing agent, and a dissolution assisting agent.

The present immune cell not only has an effect of improving cell proliferation of the immune cell but also has a high therapeutic effect as an immune cell for treatment as described in Examples below. Therefore, the present immune cell can be advantageously applied to a pharmaceutical to be used in a method such as adoptive immune cell therapy.

Herein, “to improve cell proliferation of an immune cell” means that cell survival rate reduction and/or cell proliferation efficiency degradation caused when an immune cell is cultured in the absence of a cytokine is improved, and/or cell survival rate reduction and/or cell proliferation efficiency degradation caused in an immune cell in vivo after administration of the immune cell to a living body is improved.

The present immune cell is produced by introducing a present vector into an immune cell population expected to show a therapeutic effect or preventive effect against a disease that a subject requiring treatment or prevention of the disease endogenously had, and the resultant present immune cell is returned to the living body of the subject, and thus, the disease can be treated or prevented. An example of the immune cell population derived from a subject usable in such treatment or prevention includes a T cell or an NK cell having activity to attack a treatment target of a cancer cell, for example, in a cancer patient (for example, a cell population expressing a TCR for a surface antigen of the cancer).

In the present immune cell, it is preferable that a peptide/polypeptide for treatment or prevention is expressed in addition to a present polypeptide. A present immune cell expressing the peptide/polypeptide for treatment or prevention contains, in addition to the present polynucleotide, a polynucleotide encoding the peptide/polypeptide for treatment or prevention. Since the present immune cell expressing the peptide/polypeptide for treatment or prevention is improved in cell proliferation owing to the effect of the present polypeptide, the therapeutic effect or the preventive effect of the peptide/polypeptide for treatment or prevention expressed therein can be retained, and/or enhanced.

The peptide/polypeptide for treatment or prevention applied in the present invention is not especially limited, and examples include a monoclonal antibody against a target molecule present in a cell of the treatment or prevention target; a chimeric antigen receptor (CAR) containing a single chain antibody (scFv) against the target molecule, a transmembrane domain, and an ITAM intracellular signaling region; a chimeric autoantibody receptor (CAAR) containing an antigen recognizing an autoantibody causing an autoimmune disease, a transmembrane domain, and an ITAM intracellular signaling region; a TCR; a bioactive peptide; a cytokine; a chemokine; an enzyme for enzyme replacement therapy, and since the effect has been verified in Examples below, a preferable example includes a CAR.

Examples of the bioactive peptide include medically applied peptides such as growth hormone (GH) peptide, parathyroid gland hormone (PTH) peptide, erythropoietin (EPO) peptide, glucagon-like peptide-1 receptor (GLP-1R) ligand peptide, natriuretic peptide (such as atrial natriuretic peptide [ANP], brain natriuretic peptide [BNP], and C-type natriuretic peptide [CNP]).

Examples of the enzyme for enzyme replacement therapy include alglucosidase alfa, agalsidase alfa, agalsidase beta, imiglucerase, and velaglucerase alfa.

The disease to be treated or prevented by a present pharmaceutical (present immune cell) can be appropriately selected in accordance with the type or property of the peptide/polypeptide for treatment or prevention to be expressed in the present immune cell. For example, when the peptide/polypeptide for treatment or prevention is a CAR, examples of the disease for applying the present pharmaceutical include cancer (such as colorectal cancer, stomach cancer, liver cancer, lung cancer, skin cancer, breast cancer, prostate cancer, bladder cancer, kidney cancer, pancreatic cancer, bile duct cancer, lymphoma, and leukemia). When the peptide/polypeptide for treatment or prevention is a CAAR, examples of the disease for applying the present pharmaceutical include autoimmune diseases (such as myasthenia gravis, rheumatism, multiple sclerosis, neuromyelitis optica spectrum disorder, IgG4-related disease, membranous nephropathy, rapidly progressive glomerulonephritis, and dilated cardiomyopathy). When the peptide/polypeptide for treatment or prevention is a GH peptide, an example of the disease for applying the present pharmaceutical includes adult growth hormone deficiency. When the peptide/polypeptide for treatment or prevention is a PTH peptide, an example of the disease for applying the present pharmaceutical includes osteoporosis. When the peptide/polypeptide for treatment or prevention is an EPO peptide, examples of the disease for applying the present pharmaceutical include anemia, connective tissue diseases (such as chronic rheumatoid arthritis, and systemic lupus erythematosus), chronic infectious diseases (such as tuberculosis, infective endocarditis, and liver abscess), allergic diseases (such as atopic dermatitis, and psoriasis), autoimmune diseases (such as rheumatism, and multiple sclerosis), tumors (such as ovarian tumor, and melanoma), chronic renal failure, hypothyroidism, and amyotrophic lateral sclerosis (ALS). When the peptide/polypeptide for treatment or prevention is a GLP-1R ligand peptide, examples of the disease for applying the present pharmaceutical include metabolic diseases (type 2 diabetes, hypertension, dyslipidemia, and fatty liver). When the peptide/polypeptide for treatment or prevention is a natriuretic peptide, examples of the disease for applying the present pharmaceutical include heart failure (acute heart failure (AHF)), kidney damage, and myocardial fibrosis (amyloidosis). When the peptide/polypeptide for treatment or prevention is alglucosidase alfa, an example of the disease for applying the present pharmaceutical includes Pompe disease. When the peptide/polypeptide for treatment or prevention is agalsidase alfa, or agalsidase beta, examples of the disease for applying the present pharmaceutical include Fabry disease, and lysosomal disease. When the peptide/polypeptide for treatment or prevention is imiglucerase, an example of the disease for applying the present pharmaceutical includes Gaucher disease. When the peptide/polypeptide for treatment or prevention is velaglucerase alfa, examples of the disease for applying the present pharmaceutical include lysosomal disease, and Gaucher disease. The present immune cell expressing the peptide/polypeptide for treatment or prevention can satisfactorily survive and proliferate even in a living body where a high concentration of cytokine cannot be expected owing to the effect of the present polypeptide, and therefore, since the peptide/polypeptide for treatment or prevention constantly expresses in the living body, a persistent therapeutic effect or preventive effect against these diseases can be sufficiently expected. Accordingly, the subject of treatment or prevention to which the present pharmaceutical (present immune cell) is to be administered is a person affected by an arbitrary disease (patient), or a person having a risk of onset of an arbitrary disease.

A present immune cell used as the active ingredient of a present pharmaceutical can favorably survive and/or proliferate in a living body even when it is not pre-cultured for more than 10 days before administration to a subject of treatment or prevention. Therefore, the therapeutic effect and the preventive effect can be expected even when the present immune cell used in the present pharmaceutical is administered to a subject of treatment or prevention within a short period of time after the gene introduction of a present vector to an immune cell from the viewpoint of time effectiveness and cost effectiveness. Here, the upper limit of the “short period of time after the gene introduction” can be, for example, 10 days, 9 days, 8 days, 7 days, 6 days, 5 days, 4 days, 3 days, 2 days, or 1 day (24 hours) after the gene introduction, and a preferable example includes 10 days (namely, within 10 days after the gene introduction to the present immune cell). Besides, the lower limit of the period can be, for example, 0 hours, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, or 16 hours after the gene introduction.

When a present immune cell is to be administered (transplanted) to a subject (patient) requiring adoptive immune cell therapy, a donor of the immune cells and the subject of administration (recipient) may be the same (autologous transplantation), or different as in adoptive immune cell therapy (namely, adoptive immune cell therapy of allotransplantation), but adoptive immune cell therapy in which the donor and the recipient are the same (namely, adoptive immune cell therapy of autologous transplantation) is preferred from the viewpoint of avoiding graft-versus-host disease (GVHD).

When a present immune cell is administered to a subject of treatment or prevention, the number of present immune cells to be administered varies depending on the type and the degree of the disease to be treated or prevented, the race, sex, age and the like of the subject of administration, and hence cannot be generally specified, and is usually 1×10⁴ to 1×10⁹, preferably 1×10⁵ to 1×10⁸, and more preferably 1×10⁶ to 1×10⁷.

Examples of a method for administering a present immune cell include methods such as insertion with a catheter, injection into a coronary artery or vein, or directly into a tissue or organ, and injection into a vein.

### 5. Present Preparation Method

In another aspect, the present invention provides a method for selectively concentrating/preparing an immune cell population having a desired gene suitably introduced therein by conducting gene introduction of a vector containing a present polynucleotide (namely, a present vector) to an immune cell (present preparation method). An immune cell to which the present vector has been appropriately genetically introduced has an improved cell proliferation effect as compared with an immune cell failed in the gene introduction, and therefore, a selectively concentrated cell population can be prepared by culturing the resultant immune cell in vitro for a prescribed period after the gene introduction.

The present preparation method is not especially limited as long as it is a method for preparing a cell population containing a present immune cell, including the steps of (A) conducting gene introduction of a present vector to an immune cell; and (B) culturing the immune cell resulting from the gene introduction for 5 days or more after the gene introduction. Here, “5 days or more” can be, for example, 8 days or more, 10 days or more, 11 days or more, 12 days or more, 13 days or more, 14 days or more, 15 days or more, 16 days or more, 17 days or more, 18 days or more, 19 days or more, or 20 days or more, and is preferably 15 days or more for preparing a cell population containing a present immune cell with a high purity. In prevention or treatment of a disease with a present immune cell, it is not always necessary to selectively concentrate an immune cell population having a desired gene suitably introduced therein (present immune cell population), and therefore, a treatment or preventive effect can be obtained by administering a present immune cell obtained in a short period of time after the gene introduction of the present vector as described above.

In order to prepare a cell population containing a present immune cell with a high purity by the present preparation method, the immune cell resulting from the gene introduction is cultured in the step (B) preferably in the absence of a ligand protein that binds to a receptor of IL15, IL2, or IL7, wherein the binding of the ligand protein to the receptor transmits a signal similar to a binding signal of the cytokine into an immune cell via the receptor (preferably, all the present cytokines). Herein, “cell population containing a present immune cell with a high purity” means that the ratio of the number of present immune cells contained in the cell population is at least 80%, preferably at least 85%, more preferably at least 88%, further preferably at least 90%, still further preferably at least 92%, particularly preferably at least 94%, and most preferably at least 96%.

In the present preparation method, when a cell in which a gene except for a present polynucleotide is simultaneously expressed therein is to be obtained as a present immune cell, an immune cell having another gene precedently introduced therein may be used, or a present vector carrying another gene together with a present polynucleotide may be used in the step (A). According to the present preparation method, the immune cell having the another gene expressed therein can be specifically concentrated, and thus, a cell population having a high content ratio of, for example, an immune cell expressing a desired CAR can be produced.

In the step (A), when the immune cell is a T cell, the gene introduction of a present vector may be conducted before or after stimulating a PBMC containing the T cell with an anti-CD3 antibody, or simultaneously with the anti-CD3 antibody stimulation. Besides, when the T cell is a cell population having a specific property, a stimulating factor in accordance with the type and property of the cell population may be used instead of the anti-CD3 antibody. As a method for genetically introducing a present vector to an immune cell, any method may be employed as long as it is suitable for the present vector and immune cell, and examples include an electroporation method, a calcium phosphate method, a lipofection method, and a virus infection method as described above. Besides, the immune cell can be obtained by isolating from and purifying a body fluid such as blood or bone marrow aspirate, a tissue of the spleen, the thymus, or the lymph node, or an immune cell infiltrating into a cancer tissue of primary tumor, metastatic tumor, or cancerous ascites.

When a viral vector (namely, a recombinant virus) is used as a present vector for gene introduction to an immune cell, a method for infecting the immune cell with the recombinant virus is not especially limited, and can be appropriately selected in accordance with the purpose. Examples include a polybrene method (specifically, a method for infecting an immune cell with a recombinant virion using polybrene), and a RetroNectin method (specifically, a method for infecting an immune cell with a recombinant virion using a container [such as a culture plate or dish] coated with RetroNectin).

Examples of the culture fluid used in the culture of the immune cell resulting from the gene introduction in the step (B) include a culture fluid for culturing animal cells containing 0.1 to 30 (v/v) % serum (FBS, CS, or the like) (such as DMEM, EMEM, IMDM, RPMI1640, αMEM, F-12, F-10, M-199, or AIM-V), and MyeloCult H5100 medium (manufactured by STEMCELL Technologies, ST-05150). Examples of a serum-free culture fluid include a culture fluid for culturing animal cells supplemented with an appropriate amount (for example, 1 to 30%) of commercially available B27 supplement (-insulin), N2 supplement, B27 supplement, or a serum replacement such as Knockout Serum Replacement described above.

A culture temperature for the immune cell resulting from the gene introduction in the step (B) is usually in the range of about 30 to 40° C., and preferably 37° C. Besides, a CO₂ concentration during the culture is usually in the range of about 1 to 10%, and preferably about 5%. A humidity during the culture is usually in the range of about 70 to 100%, and preferably in the range of about 95 to 100%. Besides, an O₂ concentration during the culture may be a normal oxygen concentration (18 to 22% O₂), or a low oxygen concentration (0 to 10% O₂).

The purity of the present immune cell can be obtained as follows: The present immune cell is stained with an antibody against the present cytokine labeled with a fluorescent substance (such as allophycocyanin (APC), phycoerythrin (PE), FITC (fluorescein isothiocyanate), Alexa Flour 488, Alexa Flour 647, Alexa Flour 700, PE-Texas Red, PE-Cy5, or PE-Cy7). Viable cells are stained with Hoechst 33342, Hoechst 33258, or the like, or dead cells are stained with DAPI (4′,6-diamidino-2-phenylindole), TO-PRO-3 Iodide Quinolinium, 4-[3-(3-methyl-2 (3H)-benzothiazolylidene)-1-propenyl]-1-[3-(trimethylammonio)propyl]-, diiodide, LIVE/DEAD Fixable Dead Cell Stain (manufactured by Life Technology), 7-aminoactinomycin D, propidium iodide (PI), or the like. The resultant is analyzed by flow cytometry to count the number of viable cells and the number of present immune cells in the cell population, and the purity can be calculated as a ratio of the present immune cells in the viable cells.

A present kit is not especially limited as long as it is a kit including a present polynucleotide, a present polypeptide, or a present vector specified in use for “improving proliferation of an immune cell”. The kit usually includes those generally included in this type of kits, such as a carrier; a pH buffer; a stabilizer; an instruction manual; a manual describing a method for genetically introducing a present polynucleotide, a present polypeptide, or a present vector to an immune cell; and the like.

Now, the present invention will be more specifically described with reference to Examples, and it is noted that the technical scope of the present invention is not limited to these Examples. It is noted that the name of a gene described without an origin species means that a human-derived gene was used. Besides, cell culture was conducted under condition of 37° C. and 5% CO₂.

### EXAMPLES

In the following Examples, the following methods were employed as respective experimental methods.

### [Plasmid Vector for Virus Production]

For producing a lentivirus to be used in target gene introduction to a human T cell, pLVSIN IRES-BFP obtained by substituting a gene sequence encoding green fluorescent protein (ZsGreen1) contained in pLVSIN IRES-ZsGreen1 (manufactured by Takara Bio Inc., 6191) with a gene sequence encoding another fluorescent protein, BFP. A target gene was introduced into a multicloning site of the pLVSIN IRES-BFP to prepare a plasmid vector for lentivirus production. A target cell was established with pLVSIN IRES-ZsGreen1 or pLVSIN EF1α pur (manufactured by Takara Bio Inc., 6186). For producing a retrovirus to be used in target gene introduction to a human NK cell, pMSGV1 was used (the literature “Hum Gene Ther. 2005 April; 16(4): 457-72.”). A target gene was linked to self-cleaving peptide T2A (SEQ ID NO: 14), and a gene encoding GFP, and the resultant was inserted downstream of a promoter of pMSGV1 to prepare a plasmid vector for retrovirus production.

### [Preparation of Lentivirus]

Lenti-X 293 T cell line (manufactured by Takara Bio Inc., 632180) was precedently cultured to confluent in 10% fetal bovine serum (FBS)-containing Dulbecco's Modified Eagle Medium (DMEM) (manufactured by Thermo Fisher Scientific, 10566-016), and was detached with trypsin (manufactured by Thermo Fisher Scientific, 12563029) on the day of transfection, and was seeded again in a T-flask to 70% to 80% confluent. After 2 to 4 hours, the plasmid vector for lentivirus production described above, Lentiviral High Titer Packaging Mix (manufactured by Takara Bio Inc., 6194), and TransIT-293 Transfection Reagent (manufactured by Tkara Bio Inc., MIR2704) were mixed with Opti-MEM I Reduced Serum Media (manufactured by Thermo Fisher Scientific, 31985070), the resultant was incubated for 15 minutes, and then added to the Lenti-X293 T cell line. On the next day, the medium was exchanged with 10% FBS-containing DMEM medium, and the resultant was cultured for 24 hours to collect a supernatant. If necessary, after collecting the supernatant, 10% FBS-containing DMEM medium was added again, and the resultant was cultured for 24 hours to collect a supernatant again. The thus collected supernatant was filtered, Lenti-X concentrator (manufactured by Takara Bio Inc., 631232) in a ⅓ amount was added thereto, the resultant was incubated at 4° C. for 1 hour or more, and the resultant was centrifuged (1000 to 4000×g, 30 to 45 minutes) to precipitate the virus. A supernatant was removed by aspiration, the thus obtained pellet was dissolved in 5% FBS-containing AIM-V medium (manufactured by Thermo Fisher Scientific, 12055083) (hereinafter, referred to as “basal medium”), and the resultant was cryopreserved at −80° C. or −150° C. as a lentivirus concentrate.

### [Preparation of Retrovirus]

PLAT-F cell (the literature “Exp Hematol. 2003 November; 31(11); 1007-14.”) was precedently cultured to confluent in 10% FBS-containing DMEM medium, and was detached with trypsin on the day of transfection, and was seeded again in a T-flask to 70% to 80% confluent. After 2 to 4 hours, the plasmid vector for retrovirus production described above, and X-treme GENE HP DNA transfection reagent (manufactured by Roche, 6366236001) were mixed with Opti-MEM I Reduced Serum Media, the resultant was incubated for 15 minutes, and then added to the PLAT-F cell line. On the next day, the medium was exchanged with 10% FBS-containing DMEM medium, and the resultant was cultured for 24 hours to collect a supernatant. The thus collected supernatant was filtered, Retro-X concentrator (manufactured by Takara Bio Inc., 631456) in a ⅓ amount was added thereto, the resultant was incubated at 4° C. for 1 hour or more, and the resultant was centrifuged (1000 to 4000×g, 30 to 45 minutes) to precipitate the retrovirus. A supernatant was removed by aspiration, the thus obtained pellet was dissolved in MyeloCult H5100 (manufactured by STEMCELL Technologies, ST-05150) to prepare a retrovirus concentrate.

### [Preparation of Target Gene Expressing T Cell]

PBS (phosphate buffered saline) was used to dilute an anti-CD3 antibody (manufactured by Biolegend, 317347) to 5 to 10 μg/mL, and RetroNectin (manufactured by Takara Bio Inc., T100B) to 20 to 100 μg/mL to be added to a Non-Treatment well dish. After allowing the dish to stand still at room temperature for 2 hours, or at 4° C. overnight, the resultant was washed with PBS to prepare a coated plate (hereinafter, referred to as a “CD3 Ab/RetroNectin coated plate”). A virus concentrate was added to the plate, and the resultant was centrifuged at 2000×g for 2 hours to prepare a virus binding plate. A basal medium supplemented with IL2 (manufactured by Nipro, 87-890, or manufactured by Kyowa Pharmaceutical Industry Co., Ltd., 58697900) at a final concentration of 100 U/mL (hereinafter, referred to as an “IL2 medium”) was used to suspend human-derived peripheral blood mononuclear cell (PBMC; manufactured by Cellular Technology Limited, CTL-UP1) therein at 1 to 2×10⁶ cells/mL, and the resultant was added to the virus binding plate, followed by centrifugation at 300×g for 3 to 5 minutes. In some tests, the lentivirus concentrate and PBMC were mixed in a CD3 Ab/RetroNectin coated plate, followed by centrifugation at 1000×g for 1 hour. From day 3 of the culture, three types of media (the basal medium, the IL2 medium, and a basal medium supplemented with IL15 [manufactured by Miltenyi Biotec, 130-095-762] in 10 ng/mL [hereinafter, referred to as the “IL15 medium” ]) were used to exchange the whole amount of the medium every 1 to 3 days. In preparation of a T cell expressing a membrane-bound cytokine, the basal medium was used for medium exchange from day 3 of the culture. If necessary, collected cells were suspended in CELLBANKER1 (manufactured by Takara Bio Inc., CB011) in 0.5 to 1×10⁷ cells/mL, followed by cryopreservation at −80° C. or −150° C. When a cryopreserved cell was to be used for an experiment, the thawed cell was cultured overnight in the IL2 medium before use.

### [Preparation of Target Gene Expressing NK Cell]

An anti-CD16 antibody (manufactured by Biolegend, 302050) was diluted with PBS to 5 μg/mL, and the resultant was added to a Non-Treatment well dish. The resultant was allowed to stand still at room temperature for 2 hours or at 4° C. overnight, and then washed with PBS, and PBMC, which was suspended in MyeloCult H5100 medium supplemented with IL2 and IL18 (manufactured by R&D Systems, 9124-IL) respectively in 100 U/mL and 100 ng/mL (hereinafter, referred to as the “NK medium”) was added thereto. After 5 days, the resultant cell was collected, and a CD56-positive cell (namely, an NK cell) was purified with CD56 MicroBeads (manufactured by Miltenyi Biotec, 130-050-401). PBS was used to dilute the anti-CD16 antibody to 5 μg/mL, and RetroNectin to 20 μg/mL to be added to a Non-Treatment well dish. The resultant dish was allowed to stand still at room temperature for 2 hours, or at 4° C. overnight, and then washed with PBS to prepare a coated plate (hereinafter, referred to as the “CD16 Ab/RetroNectin coated plate”). The CD56-positive cell was suspended in the NK medium at 1 to 2×10⁶ cells/mL, and the resultant was mixed with the retrovirus concentrate, followed by centrifugation at 1000×g for 1 hour. From day 3 of the culture, MyeloCult H5100 medium was used to exchange the whole amount of the medium every 1 to 3 days.

### [Flow Cytometry]

For detecting a T cell (namely, a CD3-positive cell), a fluorescent labeled anti-CD3 antibody (Alexa Flour 488 anti-human CD3 antibody [manufactured by Biolegend, 317310]) was used. For detecting an NK cell (namely, a CD56-positive CD3-negative cell), a fluorescent labeled anti-CD56 antibody (APC anti-human CD56 antibody [manufactured by Biolegend, 318310]), and a fluorescent labeled anti-CD3 antibody (APC/Cy7 anti-CD3 antibody [manufactured by Biolegend, 300426]) were used. For detecting 8 membrane-bound cytokines (IL15-IL15Rα, IL2TM, IL4TM, IL6TM, IL7TM, IL15TM, IL9TM, and IL21TM), a fluorescent labeled anti-CD215 antibody (PE anti-human CD215 antibody [manufactured by Biolegend, 330208]), and 7 biotinylated antibodies (a biotinylated anti-IL2 antibody [Biotinylated Anti-Human IL2 antibody [manufactured by Peprotech, 500-P22BT]], a biotinylated anti-IL4 antibody [Biotinylated Anti-Human IL4 antibody [manufactured by Peprotech, 500-P24BT]], a biotinylated anti-IL6 antibody [Biotinylated Anti-Human IL6 antibody [manufactured by Peprotech, 500-P26BT]], a biotinylated anti-IL7 antibody [Biotinylated Anti-Human IL7 antibody [manufactured by Peprotech, 500-P27BT]], a biotinylated anti-IL15 antibody [Biotinylated Anti-Human IL15 antibody [manufactured by Peprotech, 500-P15BT]], a biotinylated anti-IL9 antibody [Biotinylated Anti-Human IL9 antibody [manufactured by Peprotech, 500-P29BT]], and a biotinylated anti-IL21 antibody [Biotinylated Anti-Human IL21 antibody [manufactured by Peprotech, 500-P21BT]] were used. For detecting a DYKDDDDK (FLAG) tag, a fluorescent labeled anti-FLAG antibody (PE/Cy7anti-DYKDDDDK Tag antibody [manufactured by Biolegend, 637324]) was used. The fluorescent labeled antibody or the biotinylated antibody was suspended in FACS Buffer (PBS containing 0.5% BSA, 2 mM EDTA, and 0.09% Azide) to 1 to 10 μg/mL, and the resultant was added to the cell to cause a reaction at 4° C. for 15 minutes. Thereafter, the resultant was washed with FACS buffer. For detecting the biotinylated antibody, fluorescent labeled streptavidin (APC/Fire 750 Streptavidin [manufactured by Biolegend, 405250]) was diluted 100-fold with FACS Buffer, the resultant was added to the cell, the resultant was incubated at 4° C. for 15 minutes, followed by washing with FACS buffer. The thus obtained antibody stained cell was suspended in FACS Buffer containing 1 to 10 μg/mL of 7-aminoactinomycin D (7-AAD; manufactured by WAKO, 016-25241), and the resultant was subjected to measurement with a flow cytometer (manufactured by Miltenyi Biotec, MACSQuant Analyzer 10, 130-096-343). After extraction as an FCS file, FLOW JO software (manufactured by FLOWJO LLC, VER.10.7.1) was used to select a cell fraction in FSC/SSC plot, and a 7-aminoactinomycin D-negative/BFP-positive cell group was counted as a viable transfected T cell.

### [T Cell Proliferation Test Under Cytokine-Free Condition]

This test was performed by adding PBMC suspended in the IL2 medium to a CD3 Ab/RetroNectin coated plate, adding a lentivirus concentrate thereto to cause infection with a lentivirus expressed by each molecule, and after the infection, exchanging the medium with the basal medium every 1 to 3 days. With the day of starting the test defined as day 0, samples were obtained over time, and in accordance with the method described above in [Flow Cytometry], the number of viable transfected T cells (7-aminoactinomycin D-negative/BFP-positive cells) was counted. The total number of viable transfected T cells was calculated assuming that the amount of sampled liquid was a total liquid amount. Besides, in data of change over time, since the number of cells was reduced owing to the sampling, data of the number of counted cells was multiplied by a dilution ratio corresponding to the reduction to calculate the total number of viable transfected T cells.

### [NK Cell Proliferation Test Under Cytokine-Free Condition]

This test was performed by adding a CD56-positive cell suspended in an NK medium to a CD16 Ab/RetroNectin coated plate, adding a retrovirus concentrate thereto to cause infection with a retrovirus expressed by each molecule, and after the infection, exchanging the medium with the MyeloCult H5100 medium every 1 to 3 days. With the day of starting the test defined as day 0, samples were obtained over time, and in accordance with the method described above in [Flow Cytometry], the number of viable transfected NK cells (namely, 7-aminoactinomycin D-negative, CD3-negative, CD56-positive, and GFP-positive cells) was counted. The total number of viable transfected NK cells was calculated assuming that the amount of sampled liquid was a total liquid amount. Besides, in data of change over time, since the number of cells was reduced owing to the sampling, data of the number of counted cells was multiplied by a dilution ratio corresponding to the reduction to calculate the total number of viable transfected NK cells.

### [Evaluation of Cytotoxic Activity]

In order to establish ZsGreen1 transfected RAJI cell, first, pLVSIN-IRES-ZsGreen1 was used to produce a lentivirus in accordance with the method described above in [Preparation of Lentivirus]. The thus produced lentivirus was used to infect CD19-positive RAJI cell line (obtained from ATCC, CCL-86) by a usual method, and the resultant was cloned by a limiting dilution method to establish ZsGreen1 transfected RAJI cell line. For culturing the RAJI cell line, 10% FBS-containing RPMI (Roswell Park Memorial Institute) 1640 medium (manufactured by Thermo Fisher Scientific, 61870-036) was used. In order to establish CD19 expressing HeLa cell line (CD19+HeLa cell line), first, a gene encoding a polypeptide in which a CD8α-derived signal peptide (SEQ ID NO: 4), a CD19-derived extracellular domain and transmembrane domain (SEQ ID NO: 25), a spacer (GSG), a self-cleaving peptide T2A (SEQ ID NO: 14), and GFP were linked was introduced into pLVSIN EF1α pur by the method described above in [Preparation of Lentivirus] to produce a lentivirus. The thus produced lentivirus was used to infect HeLa S3 cell line (obtained from ATCC, CCL-2.2) by a usual method, and the resultant was cloned by a limiting dilution method to establish CD19+HeLa cell line. For culturing the HeLa cell line, 10% FBS-containing DMEM medium (manufactured by Thermo Fisher Scientific, 10566-016) was used. To a 96 well plate, 1×10⁴ T cells to be evaluated for cytotoxic activity were added, and an equivalent amount of (namely, 1×10⁴) RAJI cell line or CD19+HeLa cell line were added respectively to independent wells, followed by further culturing. For the entire culture, the basal medium was used. After 3 days, a half amount of the T cells was subjected to analysis for 3 fluorescent signals (ZsGreen1, GFP, and BFP) by the method described above in [Flow Cytometry], and 1×10⁵ RAJI cells, or 2×10⁴ CD19+HeLa cells were added to the remaining half amount of the T cells, followed by further culturing. After 7 days, 12 days, and 14 days, a half amount of the T cells was separated by the same method, and the number of viable RAJI cells (7-aminoactinomycin D-negative ZsGreen1-positive cells), the number of viable CD19+HeLa cells (7-aminoactinomycin D-negative GFP-positive cells), and the number of viable CAR-T cells (7-aminoactinomycin D-negative BFP-positive cells) were counted by the method described above in [Flow Cytometry]. The cytotoxic activity was shown as the number of remaining target cells assuming that the number of target cells in a well to which CAR-T cell was not added was 100%.

### [In Vivo Proliferation Test]

As a severely immunodeficient mouse, two mice (NOD/Shi-scid-IL2R γ null [NOG mouse], and NOD.Cg-Prkdc<scid>IL2 rd<tmlSug>B2m<emlTac>H2-Ab1<tm1Doi>/Jic [NOG-ΔMHC mouse]) were used. Three T cells (BFP expressing control T cell, “IL15TM-CD137” expressing T cell, and “IL15TM-HVEM” expressing T cell) were administered to 5 week old (male) mice of the 2 mice through the tail vein. After 9 to 14 days, the mice were euthanized, and the spleen was collected, and mechanically distributed, and the resultant was hemolyzed with RBC Lysis buffer (manufactured by Thermo Fisher Scientific, 00-4333-57). The thus obtained cell suspension was sampled, and FACS Buffer containing 10 μg/mL of 7-aminoactinomycin D was added thereto, and the number of viable transfected T cells (7-aminoactinomycin D-negative BFP-positive cells) was counted by the method described above in [Flow Cytometry].

### [Evaluation of Antitumor Activity]

pLVSIN EF1α pur into which luciferase gene had been introduced was used to produce a lentivirus in accordance with the method described above in [Preparation of Lentivirus], and then, the resultant was used to infect the CD19+HeLa cell line to establish luciferase expressing CD19+HeLa cell line. The resultant cell was suspended in PBS, and 1×10⁶ luciferase expressing CD19+HeLa cells were subcutaneously administered to the armpit of a 6 week old (female) NOG mouse to produce a tumor-bearing mouse model. After 11 days, two CAR-T cells (“CAR (CD137-CD3ζ)” expressing T cell, and “CAR (CD3ζ)/IL15TM-OX40” expressing T cell) each in an amount of 1×10⁵ cells were administered to the jugular vein. Luciferin (manufactured by Promega, P1041) was administered over time, and the level of luciferin-derived luminescence was measured with FUSION FX7. EDGE (manufactured by Vilber Lourmat).

### Example 1: T Cell Proliferation Under Cytokine-Free Condition is Insufficient in Conventional Technique

An immune cell for treatment such as a T cell used in adoptive immune cell therapy needs to proliferate or survive in a living body for exhibiting required functions. A T cell is known to require a cytokine, such as IL15, IL2, or IL7, in culture in vitro, and is generally cultured with such a cytokine added in a concentration of 1 to 10 ng/mL. In a living body, however, it has been reported that the concentration of such a cytokine is extremely low (Non Patent Literatures 3 and 4). Therefore, it is presumed that the T cell proliferation observed in culture in vitro can be realized under a condition of a high concentration of cytokine, but cannot be expected in vivo (in a living body). For solving this problem, a method in which IL15 is tethered on a cell membrane is known as a method for increasing the T cell proliferation (Non Patent Literatures 5 and 6, and Patent Literature 3). In order to examine whether such a method can impart sufficient cell proliferation also under a cytokine-free condition presumed in a living body, it was decided to evaluate in vitro cell proliferation in a cytokine-free medium.

In the present example, a method in which a T cell contained in a PBMC was genetically introduced while reacting with an anti-CD3 antibody was employed. First, a ratio of T cells in proliferated cells was checked. PBMCs respectively derived from six donors, and suspended in IL2 medium were added to CD3 Ab/RetroNectin coated plates, and infected with a lentivirus prepared using pLVSIN IRES-BFP (control vector). After 3 days and 7 days, resultant cells were collected, and subjected to antibody staining using a BFP fluorescent signal and a fluorescent labelled anti-CD3 antibody in accordance with the method described above in [Flow Cytometry], and thus, a ratio of CD3 positive cells (namely, T cells) in BFP expressing cells was evaluated.

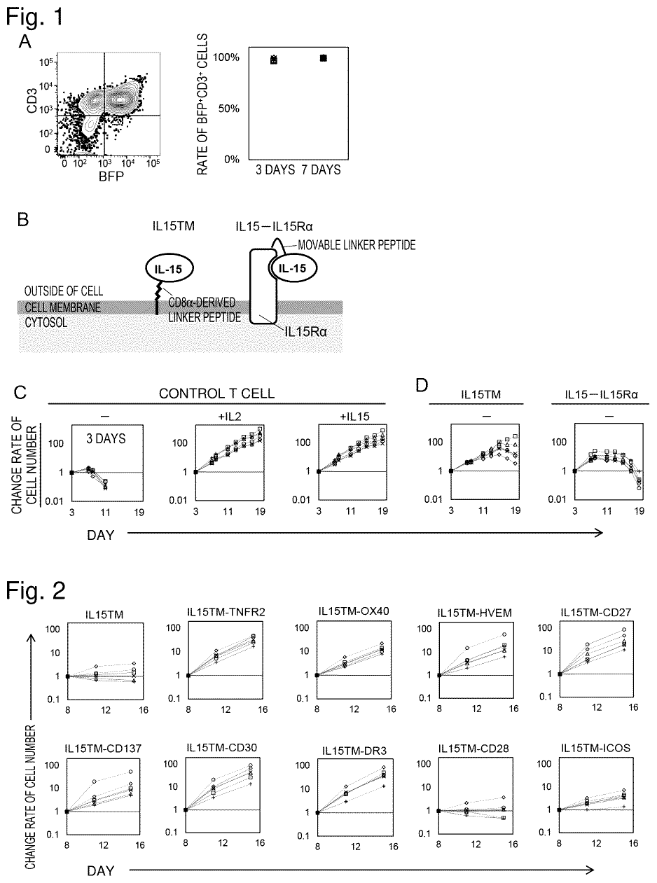

As a result, the ratios of BFP positive cells and CD positive cells were, in terms of a median (first quartile-third quartile), respectively 97.8% (97.5%-98.8%), and 99.6% (99.4%-99.8%), and thus, it was revealed that 99% of the BFP positive (namely, transfected) cells were CD3 positive (namely, T cells) (FIG. 1A). In other words, it was confirmed that gene expressing cells obtained after day 3 of the culture can be regarded as T cells.

Next, cell proliferation ability obtained by culturing, in a cytokine-free medium, a T cell expressing a cytokine tethered to a cell membrane was evaluated. Herein, a cytokine tethered to a cell membrane (namely, a membrane-bound cytokine) is referred to using TM following the gene name (gene symbol) of the cytokine in some cases (as, for example, IL15TM), and a general cytokine not tethered to a cell membrane is referred to using “s” followed by the gene name of the cytokine (as, for example, sIL15).

In order to produce a T cell expressing IL15TM (FIG. 1B) used as the membrane-bound cytokine, a gene encoding a polypeptide (SEQ ID NO: 1) having an IL2-derived signal peptide (SEQ ID NO: 3) added to the N terminal of IL15TM was introduced into a multicloning site of pLVSIN IRES-BFP to produce an expression vector. Besides, in order to produce an “IL15-IL15Rα” (FIG. 1B) expressing T cell, a gene encoding a polypeptide (SEQ ID NO: 2) having an IL2-derived signal peptide (SEQ ID NO: 3) added to the N terminal of “IL15-IL15Rα” was introduced into a multicloning site of pLVSIN IRES-BFP to produce an expression vector. The thus produced expression vector was used to produce a lentivirus in accordance with the method described above in [Preparation of Lentivirus]. PBMCs derived from six donors, and suspended in IL2 medium were respectively added to CD3Ab/RetroNectin coated plates, and the PBMCs were infected with the produced lentivirus in accordance with the method described above in [Preparation of Target Gene Expressing T Cell]. On day 3 of the culture (namely, 3 days after the infection), the resultant cells were transferred to a basal medium to be cultured therein, and thus, an IL15TM expressing T cell and an “IL15-IL15Rα” expressing T cell were prepared. Besides, as a control, a lentivirus containing an empty vector (pLVSIN IRES-BFP) was used to infect PBMCs of the six donors, and from day 3 of the culture, the resultant cells were transferred to three media (basal medium, IL2 medium, and IL15 medium) to be cultured therein to prepare BFP expressing control T cells. In accordance with the method described above in [T Cell Proliferation Test under Cytokine-free Condition], sampling was performed over time to count the number of BFP positive T cells with a flow cytometer, and a change rate of cell number was evaluated as magnification change based on the number on day 3 of the culture.

As a result, it was found that the number of the BFP expressing control T cells was reduced in the basal medium from day 7, but persistently proliferated in IL2 medium and IL5 medium (FIG. 1C). On the other hand, the IL15TM expressing T cells and “IL15-IL15Rα” expressing T cells derived from many of the donors stopped to proliferate in the basal medium on day 11 to 14 of the culture (FIG. 1D).

This result reveals that cell proliferation equivalent to that obtained by cytokine addition cannot be imparted under a cytokine-free condition presumed in a living body only by expressing IL15TM or “IL15-IL15R06” in an immune cell (T cell).

TABLE 1
Change Rate of Cell Number of Conventional Membrane-
bound IL15 Expressing T Cell in Cytokine-free

| Expressed | | Median of Change Rate of Cell Number at Culture End | |
|---|---|---|---|
| Molecule | Culture Medium | Point (First Quartile-Third Quartile) | |
| BFP | Basal Medium | <0.1 | |
| BFP | IL2 Medium | 411.2 | (221.5-536.8) |
| BFP | IL15 Medium | 181.2 | (141.8-321.4) |
| IL15TM | Basal Medium | 14.5 | (10.3-60.4) |
| IL15-IL15Rα | Basal Medium | 0.2 | (0.1-0.3) |

### Example 2: A T Cell Proliferates Under a Cytokine-Free Condition when a TNFRSF Molecule-Derived Intracellular Domain is Linked to Membrane-Bound IL15

In activation of a T cell, antigen non-specific co-stimulation (auxiliary stimulation) for CD28 or CD137, or cytokine is known to play a significant role as a signal assisting antigen-specific stimulation for a TCR/CD3 complex. The role of co-stimulation in proliferation function of a cytokine has not, however, been examined. The present inventors have considered that CD28 or CD137 may assist the function of a cytokine, and have examined a combination thereof with membrane-bound IL15.

A gene encoding a polypeptide, in which a CD8α-derived linker peptide (SEQ ID NO: 15), a CD8α-derived transmembrane domain (SEQ ID NO: 16), and an intracellular domain derived from any one of nine co-stimulatory molecules (seven TNFRSF molecules [TNFR2 [SEQ ID NO: 5], OX40 [SEQ ID NO: 6], HVEM [SEQ ID NO: 7], CD27 [SEQ ID NO: 8], CD137 [SEQ ID NO: 9], CD30 [SEQ ID NO: 10], and DR3 [SEQ ID NO: 11]], or any one of two CD28 family molecules [CD28 [SEQ ID NO: 12] and ICOS [SEQ ID NO: 13]] were linked to the C terminal side of IL15 (SEQ ID NO: 22) having an IL2-derived signal peptide (SEQ ID NO: 3) added to the N terminal side, was introduced into a multicloning site of pLVSIN IRES-BFP to produce an expression vector, and a lentivirus was produced in accordance with the method described above in [Preparation of Lentivirus]. PBMCs derived from six donors, and suspended in IL2 medium were respectively added to CD3 Ab/RetroNectin coated plates, and the PBMCs were infected with the produced lentivirus in accordance with the method described above in [Preparation of Target Gene Expressing T Cell], and the resultant was cultured in a basal medium from day 3 of the culture. Besides, a BFP expressing control T cell, and an IL15TM expressing T cell, used as a control group, were also produced by the same method as that described in Example 1, and similarly cultured. In accordance with the method described above in [T Cell Proliferation Test under Cytokine-free Condition], the sampling was performed over time, and the number of BFP positive cells was counted with a flow cytometer. As the test result, a change rate of cell number was evaluated as magnification change based on the number on day 8 of the culture when the proliferation of the BFP expressing control T cell stopped.

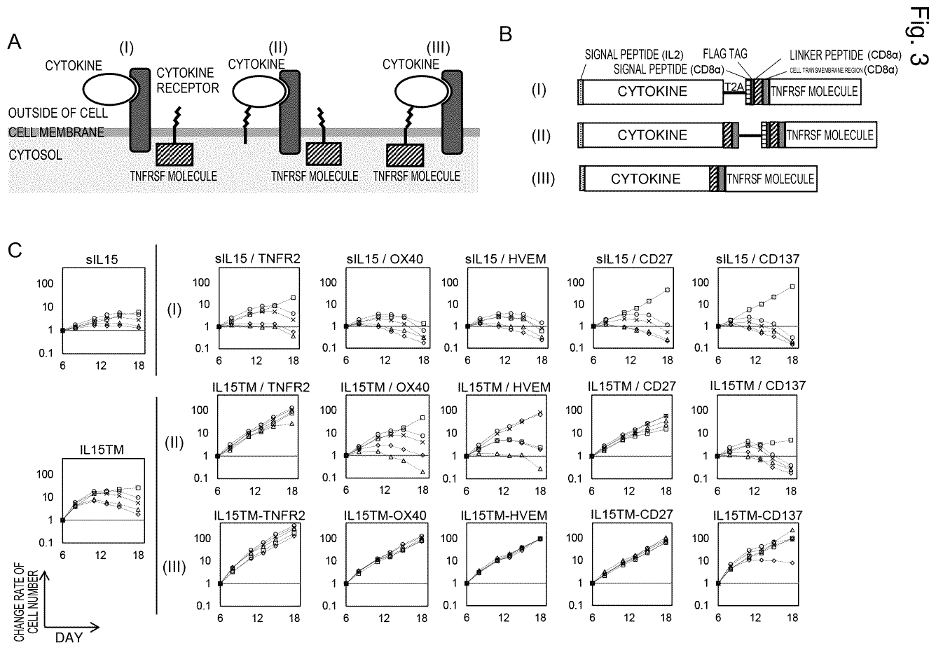

As a result, when the membrane-bound IL15 (IL15TM-TNFR2, IL15TM-OX40, IL15TM-HVEM, IL15TM-CD27, IL15TM-CD137, IL15TM-CD30, and IL15TM-DR3) linked to the intracellular domains derived from the seven TNFRSF molecules (TNFR2, OX40, HVEM, CD27, CD137, CD30, and DR3) were expressed in T cells, the cell proliferation efficiency was improved in all the donors as compared with the case in which the conventional membrane-bound IL15 was expressed, and was higher by 5 to 24 times in terms of the median (FIG. 2 and Table 2). On the other hand, when the membrane-bound IL15 (IL15TM-CD28 and IL15TM-ICOS) linked to the intracellular domains derived from the two CD28 family molecules (CD28 and ICOS) were expressed in T cells, the cell proliferation efficiency was improved only by about twice at most as compared with the case in which the conventional membrane-bound IL15 was expressed. In other words, all the molecules that sufficiently improved the cell proliferation efficiency were molecules belonging to the TNFRSF, and all the molecules that were weak in the improvement level were molecules belonging to the CD28 family.

This result reveals the following: When membrane-bound IL15 in a state linked to a TNFRSF molecule-derived intracellular domain is expressed in an immune cell, the cell proliferation under a cytokine-free culture condition is improved as compared with that in an immune cell in which only membrane-bound IL15 is expressed, or membrane-bound IL15 in a state linked to an intracellular domain derived from a CD28 family molecule, that is, a co-stimulatory molecule except for the TNFRSF molecule. A method in which an anti-CD28 antibody is added for the purpose of improving the proliferation of a T cell in culture in vitro is widely known, and CD28 or CD137 is generally used as a CAR in many cases, and therefore, it was surprising that a signal of not a CD28 family molecule but a TNFRSF molecule assists the proliferation of a T cell using a cytokine.

TABLE 2
Change Rate and Improvement Rate of Cell Number of Membrane-bound IL15 Expressing
T Cell Linked to Co-stimulatory Molecule-derived Intracellular Domain

| Expressed | Median of Change Rate of Cell Number at Culture End | | Improvement Rate | |
|---|---|---|---|---|
| Molecule | Point (First Quartile-Third Quartile) | | (Control Group) | |
| IL15TM | 1.8 | (0.3-1.9) | | |
| IL15TM-TNFR2 | 31.5 | (25.9-43.4) | 100% | (IL15TM) |
| IL15TM-OX40 | 12.5 | (10.4-13.9) | 100% | (IL15TM) |
| IL15TM-HVEM | 15.2 | (12.0-20.0) | 100% | (IL15TM) |
| IL15TM-CD27 | 23.3 | (18.7-42.0) | 100% | (IL15TM) |
| IL15TM-CD137 | 9.4 | (6.6-14.8) | 100% | (IL15TM) |
| IL15TM-CD30 | 43.5 | (30.2-61.7) | 100% | (IL15TM) |
| IL15TM-DR3 | 39.2 | (35.9-48.8) | 100% | (IL15TM) |
| IL15TM-CD28 | 1.2 | (0.7-1.3) | 50% | (IL15TM) |
| IL15TM-ICOS | 3.9 | (3.4-4.5) | 100% | (IL15TM) |

“Improvement Rate” shown in the table means a rate of the number of donors having, at the end point, a higher change rate of cell number than that of a control group, and having a change rate of cell number of 1 or more.

### Example 3: A TNFRSF Molecule Functions Through Co-Expression with Membrane IL15

In order to evaluate whether, for improving cell proliferation under a cytokine-free culture condition, a TNFRSF molecule-derived intracellular domain needs to be physically linked to membrane-bound IL15, or it is sufficient, without needing such physical linkage, to simply express a TNFRSF molecule-derived intracellular domain and membrane-bound IL15 simultaneously in a cell, three expression pattern types of a cytokine and a TNFRSF molecule (FIG. 3A) were analyzed for the change rate of cell number by expressing the respective molecules and fusion protein. These three types were specifically a type (I) in which secreted IL15 and a TNFRSF molecule-derived intracellular domain are independently expressed; a type (II) in which membrane-bound IL15 and a TNFRSF molecule-derived intracellular domain are independently expressed; and a type (III) in which membrane-bound IL15 and a TNFRSF molecule-derived intracellular domain are linked to be expressed as a fusion protein.

In order to produce a T cell expressing the molecule of the type (I), a gene encoding a polypeptide, in which an IL2-derived signal peptide (SEQ ID NO: 3) was added to the N terminal side of IL15 (SEQ ID NO: 22), and a spacer (GSG), self-cleaving peptide T2A (SEQ ID NO: 14), a CD8α-derived signal peptide (SEQ ID NO: 4), FLAG tag sequence (SEQ ID NO: 28), a CD8α-derived linker peptide (SEQ ID NO: 15), a CD8α-derived transmembrane domain (SEQ ID NO: 16), and an intracellular domains derived from any one of five TNFRSF molecules (TNFR2, OX40, HVEM, CD27, and CD137) (SEQ ID NOs: 5 to 9) were successively linked to the C terminal side, was introduced into a multicloning site of pLVSIN IRES-BFP to produce an expression vector (FIG. 3B). These polypeptides to be expressed are respectively referred to as sIL15/TNFR2, sIL15/OX40, sIL15/HVEM, sIL15/CD27, and sIL15/CD137 in some cases.

In order to produce a T cell expressing the molecule of the type (II), a gene encoding a polypeptide, in which an IL2-derived signal peptide (SEQ ID NO: 3) was added to the N terminal side of IL15 (SEQ ID NO: 22), and a CD8α-derived linker peptide (SEQ ID NO: 15), a CD8α-derived transmembrane domain (SEQ ID NO: 16), a spacer (GSG), self-cleaving peptide T2A (SEQ ID NO: 14), a CD8α-derived signal peptide (SEQ ID NO: 4), FLAG tag sequence (SEQ ID NO: 28), a CD8α-derived linker peptide (SEQ ID NO: 15), a CD8α-derived transmembrane domain (SEQ ID NO: 16), and an intracellular domain of any one of the five TNFRSF molecules (SEQ ID NOs: 5 to 9) were successively linked to the C terminal side, was introduced into a multicloning site of pLVSIN IRES-BFP to produce an expression vector (FIG. 3B). These polypeptides to be expressed are respectively referred to as IL15TM/TNFR2, IL15TM/OX40, IL15TM/HVEM, IL15TM/CD27, and IL15TM/CD137 in some cases.

In order to produce a T cell expressing the fusion protein of the type (III), a gene encoding a polypeptide, in which an IL2-derived signal peptide (SEQ ID NO: 3) was added to the N terminal side of IL15 (SEQ ID NO: 22), and a CD8α-derived linker peptide (SEQ ID NO: 15), CD8α-derived transmembrane domain (SEQ ID NO: 16), and an intracellular domain derived from any one of the five TNFRSF molecules (SEQ ID NOs: 5 to 9) were successively linked to the C terminal side of IL15 (SEQ ID NO: 22), was introduced into a multicloning site of pLVSIN IRES-BFP to produce an expression vector (FIG. 3B). These polypeptides to be expressed are respectively referred to as IL15TM-TNFR2, IL15TM-OX40, IL15TM-HVEM, IL15-TMCD27, and IL15-TMCD137 in some cases.

The thus produced expression vectors were used to produce various lentiviruses in accordance with the method described above in [Preparation of Lentivirus]. PBMCs derived from five donors, and suspended in IL2 medium were respectively added to CD3 Ab/RetroNectin coated plates, and the PBMCs were infected with the produced lentiviruses in accordance with the method described above in [Preparation of Target Gene Expressing T cell], and the resultant was cultured in a basal medium from after 3 days. Besides, a BFP expressing control T cell, and an sIL15 expressing T cell and an IL15TM expressing T cell, used as control groups, were also produced by the same method as that described in Example 1, and similarly cultured. In accordance with the method described above in [T Cell Proliferation Test under Cytokine-free Condition], the sampling was performed over time, and the number of BFP positive cells was counted with a flow cytometer. As the test result, a change rate of cell number was evaluated as magnification change based on the number on day 6 of the culture when the proliferation of the BFP expressing control T cell stopped.

As a result, the proliferation efficiency of the T cell expressing the molecule of the type (I) (namely, in which secreted IL15 and the TNFRSF molecule-derived intracellular domain were independently expressed) was 1 time or less in terms of the median in most of the donors as compared with the proliferation efficiency of the T cell expressing secreted IL15. On the other hand, the proliferation efficiency of the T cell expressing the molecule of the type (II) (namely, in which IL15TM and the TNFRSF molecule-derived intracellular domain were independently expressed) was varied from 1 time or less to 16 times depending on the donor and the type of the TNFRSF molecule derived intracellular domains to be combined as compared with the proliferation efficiency of the T cell expressing IL15TM, but the cell proliferation was improved in about a half of the donors. Besides, the proliferation efficiency of the T cell expressing the fusion protein of the type (III) (namely, the fusion protein in which IL15TM and the TNFRSF molecule-derived intracellular domain were linked) was improved in all the donors as compared with the proliferation efficiency of the T cell expressing IL15TM, and was 14 times to 50 times higher (FIG. 3C, and Table 3).

These results reveal that in order for a TNFRSF molecule to function as an auxiliary signal for proliferation of an immune cell (T cell) using a cytokine, IL15 needs to be tethered not in a secreted form but in a membrane-bound form. In addition, it is revealed that the TNFRSF molecule-derived intracellular domain exhibits a prescribed effect when expressed in an immune cell independently from membrane-bound IL15, but more effectively functions when linked to membrane-bound IL15 to be expressed as a fusion protein.

TABLE 3
Change Rate and Improvement Rate of Cell Number of
T Cell Expressing Molecules of Types (I) to (III)

| Expressed | Median of Change Rate of Cell Number at Culture End | | Improvement Rate | |
|---|---|---|---|---|
| Molecule | Point (First Quartile-Third Quartile) | | (Control Group) | |
| sIL15 | 2.8 | (1.6-4.7) | | |
| sIL15/TNFR2 | 2.1 | (0.6-4.0) | 20% | (sIL15) |
| sIL15/OX40 | 0.3 | (0.3-0.6) | 0% | (sIL15) |
| sIL15/HVEM | 0.6 | (0.3-0.8) | 0% | (sIL15) |
| sIL15/CD27 | 0.5 | (0.2-1.2) | 20% | (sIL15) |
| sIL15/CD137 | 0.2 | (0.2-0.3) | 20% | (sIL15) |
| IL15TM | 5.7 | (2.9-9.8) | | |
| IL15TM/TNFR2 | 92.0 | (79.1-116.5) | 100% | (IL15TM) |
| IL15TM/OX40 | 4.0 | (1.0-7.6) | 20% | (IL15TM) |
| IL15TM/HVEM | 2.4 | (2.0-70.2) | 40% | (IL15TM) |
| IL15TM/CD27 | 36.0 | (22.0-58.8) | 80% | (IL15TM) |
| IL15TM/CD137 | 0.3 | (0.3-0.4) | 0% | (IL15TM) |
| IL15TM-TNFR2 | 282.6 | (173.3-317.8) | 100% | (IL15TM) |
| IL15TM-OX40 | 97.5 | (82.3-119.7) | 100% | (IL15TM) |
| IL15TM-HVEM | 97.7 | (97.1-100.1) | 100% | (IL15TM) |
| IL15TM-CD27 | 78.3 | (67.9-88.5) | 100% | (IL15TM) |
| IL15TM-CD137 | 93.7 | (91.3-105.8) | 100% | (IL15TM) |

“Improvement Rate” shown in the table means a rate of the number of donors having, at the end point, a higher change rate of cell number than that of a control group, and having a change rate of cell number of 1 or more.

### Example 4: Cell Proliferation of an Immune Cell is Improved by Expressing, in the Immune Cell, IL2 and IL7 as a Chimeric Ligand with a TNFRSF Molecule

As a cytokine for activating a T cell, members of the γc cytokine family except for IL15 are known in addition to IL15. Therefore, it was examined whether a T cell can be proliferated under a cytokine-free culture condition also when such a cytokine was expressed as a membrane-bound cytokine-TNFRSF molecule chimeric ligand.

In order to produce a T cell expressing a conventional membrane-bound cytokine, a gene encoding a polypeptide, in which an IL2-derived signal peptide (SEQ ID NO: 3) was added to the N terminal side of any of six cytokines (IL2 [SEQ ID NO: 17], IL4 [SEQ ID NO: 18], IL6 [SEQ ID NO: 19], IL7 [SEQ ID NO: 20], IL9 [SEQ ID NO: 21], and IL21 [SEQ ID NO: 23]), and a CD8α-derived linker peptide (SEQ ID NO: 15) and a CD8α-derived transmembrane domain (SEQ ID NO: 16) were successively linked to the C terminal side thereof, was introduced into a multicloning site of pLVSIN IRES-BFP to produce an expression vector (these polypeptides to be expressed being respectively referred to as IL2TM, IL4TM, IL6TM, IL7TM, IL9TM, and IL21TM in some cases). Besides, in order to produce a T cell expressing a membrane-bound cytokine-TNFRSF molecule chimeric ligand, a gene encoding a polypeptide, in which intracellular domains derived from two TNFRSF molecules (TNFR2 and OX40) (SEQ ID NOs: 5 and 6) were successively linked to the C terminal side of a CD8α-derived transmembrane domain of a conventional membrane-bound cytokine expression vector, was introduced into a multicloning site of pLVSIN IRES-BFP to produce an expression vector (these polypeptides to be expressed being respectively referred to as IL2TM-TNFR2, IL4TM-TNFR2, IL6TM-TNFR2, IL7TM-TNFR2, IL9TM-TNFR2, IL21TM-TNFR2, IL2TM-OX40, IL4TM-OX40, IL6TM-OX40, IL7TM-OX40, IL9TM-OX40, and IL21TM-OX40 in some cases). The thus produced expression vectors were used to produce various lentiviruses in accordance with the method described above in [Preparation of Lentivirus]. PBMCs derived from six donors, and suspended in IL2 medium were respectively added to CD3 Ab/RetroNectin coated plates, and the resultant PBMCs were infected with the produced lentiviruses in accordance with the method described above in [Preparation of Target Gene Expressing T cell], and the resultant was cultured in a basal medium from after 3 days. Besides, a BFP expressing control T cell, and six membrane-bound cytokine expressing T cells used as a control group (IL2TM expressing T cell, IL4TM expressing T cell, IL6TM expressing T cell, IL7TM expressing T cell, IL9TM expressing T cell, and IL21TM expressing T cell) were also produced by the same method as that described in Example 1, and similarly cultured. In accordance with the method described above in [T Cell Proliferation Test under Cytokine-free Condition], the sampling was performed over time, and the number of BFP positive cells was counted with a flow cytometer. As the test result, a change rate of cell number was evaluated as magnification change based on the number on day 8 of the culture when the proliferation of the BFP expressing control T cell stopped.

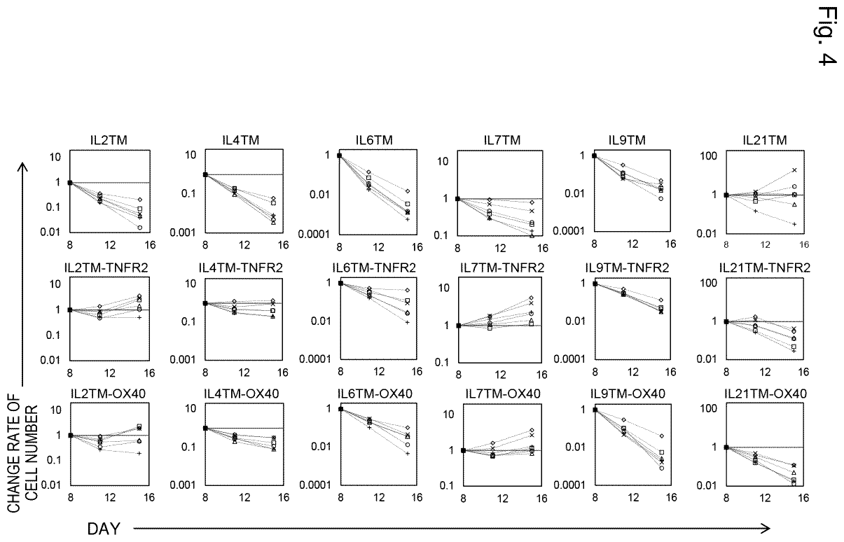

As a result, when the five membrane-bound cytokines (IL2TM, IL4TM, IL6TM, IL7TM, and IL9TM) were expressed in a T cell, the change rate of cell number at the culture end point was 1 or less in any of the cytokines, and the cell number could not be retained (FIG. 4, and Table 4). On the other hand, when the three membrane-bound cytokines (IL4TM, IL6TM, and IL9TM) among these five membrane-bound cytokines were expressed in a T cell as chimeric ligands with intracellular domains derived from two TNFRSF molecules (TNFR2 or OX40), the change rate of cell number at the end point of the culture was not improved beyond 1, but when two membrane-bound cytokines (IL2TM and IL7TM) were expressed in a T cell as chimeric ligands with the intracellular domains derived from the two TNFRSF molecules, the change rate of cell number at the end point of the culture was improved beyond 1, and the cell number was retained (FIG. 4 and Table 4). Also chimeric ligands obtained by combining IL7 with the intracellular domain of HVEM, CD27, CD137, or CD30 was also found to have a similar effect of retaining the cell number.

This result reveals that when IL2 and IL7 are employed as the cytokine used in a membrane-bound cytokine-TNFRSF molecule chimeric ligand, the cell proliferation ability of an immune cell under a cytokine-free culture condition can be improved as in using IL15. It is noted that when IL21TM was expressed in a T cell as a chimeric ligand with the two TNFRSF molecule-derived intracellular domains, the change rate of cell number at the end point of the culture tended to be rather reduced to 1 or less as compared with the case in which only IL21TM was expressed in a T cell.

TABLE 4
Change Rate and Improvement Rate of Cell Number of T Cell Expressing
Molecule of Type (III) Including Various Membrane Cytokines

| | Median of Change Rate of Cell Number at Culture End | | Improvement Rate | |
|---|---|---|---|---|
| Expressed Molecule | Point (First Quartile-Third Quartile) | | (Control Group) | |
| IL2TM | <0.1 | | | |
| IL2TM-TNFR2 | 2.0 | (1.1-2.8) | 100% | (IL2TM) |
| IL2TM-OX40 | 1.3 | (0.6-1.9) | 50% | (IL2TM) |
| IL4TM | <0.1 | | | |
| IL4TM-TNFR2 | 0.4 | (0.3-0.8) | 0% | (IL4TM) |
| IL4TM-OX40 | 0.1 | (0.1-0.3) | 0% | (IL4TM) |
| IL6TM | <0.1 | | | |
| IL6TM-TNFR2 | 0.1 | (0.0-0.1) | 0% | (IL6TM) |
| IL6TM-OX40 | <0.1 | | 0% | (IL6TM) |
| IL7TM | 0.2 | (0.2-0.4) | | |
| IL7TM-TNFR2 | 2.2 | (1.6-3.6) | 100% | (IL7TM) |
| IL7TM-OX40 | 1.2 | (1.0-2.3) | 100% | (IL7TM) |
| IL9TM | <0.1 | | | |
| IL9TM-TNFR2 | <0.1 | | 0% | (IL9TM) |
| IL9TM-OX40 | <0.1 | | 0% | (IL9TM) |
| IL21TM | 1.1 | (0.5-2.3) | | |
| IL21TM-TNFR2 | 0.1 | (0.1-0.3) | 0% | (IL21TM) |

| | | | |
|---|---|---|---|
| IL21TM-OX40 | <0.1 | 0% | (IL21TM) |

“Improvement Rate” shown in the table means a rate of the number of donors having, at the end point, a higher change rate of cell number than that of a control group, and having a change rate of cell number of 1 or more.

### Example 5: A Present Membrane-Bound Cytokine-TNFRSF Molecule Chimeric Ligand Functions without Depending on a Specific Linker Peptide or a Specific Transmembrane Domain

The influence, on the proliferation ability of a T cell under a cytokine-free condition, of the type or length of a linker peptide, or the type of a transmembrane domain contained in a present membrane-bound cytokine-TNFRSF molecule chimeric ligand was evaluated. Specifically, in order to produce a T cell expressing IL15TM-TNFR2 containing a CD8α-derived linker peptide and a transmembrane domain used as the fusion protein of the type (III) described in Examples 2 to 4, and a T cell expressing IL15TM-TNFR2 containing a CD28-derived linker peptide and transmembrane domain, a gene encoding a polypeptide, in which an IL2-derived signal peptide (SEQ ID NO: 3) was added to the N terminal side of IL15 (SEQ ID NO: 22), and a CD28-derived linker peptide (SEQ ID NO: 29), a CD28-derived transmembrane domain (SEQ ID NO: 30), and a TNFR2-derived intracellular domain (SEQ ID NO: 5) were successively linked to the C terminal side of IL15 (SEQ ID NO: 22), was introduced into a multicloning site of pLVSIN IRES-BFP to produce an expression vector. Besides, as a control group, in order to produce a T cell expressing IL15TM containing a CD8α-derived linker peptide and a transmembrane domain, and a T cell expressing IL15TM containing a CD28-derived linker peptide and a transmembrane domain, a gene encoding a polypeptide, in which an IL2-derived signal peptide (SEQ ID NO: 3) was added to the N terminal side of IL15 (SEQ ID NO: 22), and a CD28-derived linker peptide (SEQ ID NO: 29), and a CD28-derived transmembrane domain (SEQ ID NO: 30) were successively linked to the C terminal side of IL15 (SEQ ID NO: 22), was introduced into a multicloning site of pLVSIN IRES-BFP to produce an expression vector. Besides, in order to produce a T cell expressing IL15TM-TNFR2 containing a CD8α-derived linker peptide consisting of 30 amino acid residues (SEQ ID NO: 31) obtained by deleting an N terminal side portion of a CD8α-derived linker peptide (SEQ ID NO: 15) consisting of 62 amino acid residues, or a CD8α-derived linker peptide (SEQ ID NO: 32) consisting of 15 amino acid residues, a gene encoding a polypeptide, in which an IL2-derived signal peptide (SEQ ID NO: 3) was added to the N terminal side of IL15 (SEQ ID NO: 22), and any of two CD8α-derived linker peptides (SEQ ID NOs: 31 and 32), a CD8α-derived transmembrane domain (SEQ ID NO: 16), and a TNFR2-derived intracellular domain (SEQ ID NO: 5) were successively linked to the C terminal side of IL15 (SEQ ID NO: 22), was introduced into a multicloning site of pLVSIN IRES-BFP to produce an expression vector. The thus produced expression vectors were used to produce various lentiviruses in accordance with the method described above in [Preparation of Lentivirus]. PBMCs derived from five donors, and suspended in IL2 medium were respectively added to CD3 Ab/RetroNectin coated plates, and the resultant PBMCs were infected with the produced lentiviruses in accordance with the method described above in [Preparation of Target Gene Expressing T cell], and the resultant was cultured in a basal medium from after 3 days. In accordance with the method described above in [T Cell Proliferation Test under Cytokine-free Condition], the sampling was performed over time, and the number of BFP positive cells was counted with a flow cytometer. Besides, a BFP expressing control T cell was similarly cultured. As the test result, a change rate of cell number was evaluated as magnification change based on the number on day 5 of the culture when the proliferation of the BFP expressing control T cell stopped.

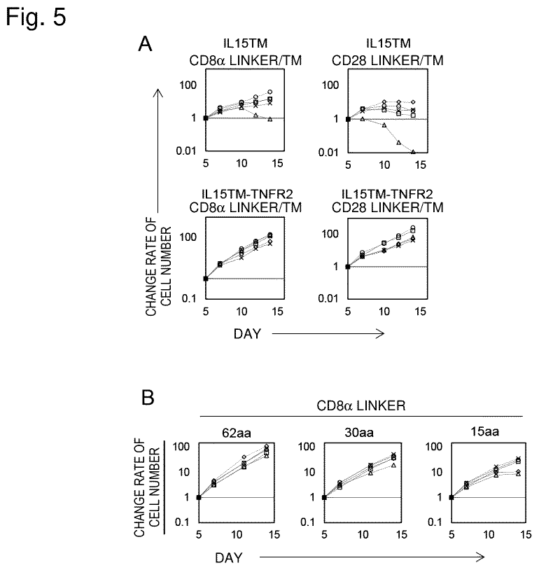

As a result, the T cell expressing IL15TM-TNFR2 containing the CD28-derived linker peptide and the transmembrane domain exhibited favorable cell proliferation in the same manner as the T cell expressing the IL15TM-TNFR2 containing the CD8α-derived linker peptide and the transmembrane domain in all the donors as compared with the BFP expressing control T cell (FIG. 5A, and Table 5).

This result reveals that a present membrane-bound cytokine-TNFRSF molecule chimeric ligand can make a contribution to improvement of proliferation ability of an immune cell under a cytokine-free culture condition regardless of the type of a linker peptide or a transmembrane domain contained in the chimeric ligand.

TABLE 5

| Expressed | | Median of Change Rate of Cell Number at Culture | Improvement |
|---|---|---|---|
| Molecule | Linker/TM | End Point (First Quartile-Third Quartile) | Rate |
| IL15TM | CD8α | 14.5(8.1-15.5) | |
| IL15TM-TNFR2 | CD8α | 120.6(65.2-135.0) | 100% |
| IL15TM | CD28 | 3.2(1.7-3.6) | |
| IL15TM-TNFR2 | CD28 | 68.5(52.5-170.8) | 100% |

“Improvement Rate” shown in the table means a rate of the number of donors having, at the end point, a higher change rate of cell number than T cells expressing membrane-bound IL15 with the same linker peptide and transmembrane domain, and having a change rate of cell number of 1 or more.

Besides, the T cells expressing IL15TM-TNFR2 respectively containing the three CD8α-derived linker peptides having different amino acid residue lengths (SEQ ID NO: 15 [62aa], SEQ ID NO: 31 [30aa], and SEQ ID NO: 32 [15aa]) all exhibited, in all the donors, more favorable cell proliferation than the BFP expressing control T cell (FIG. 5B, and Table 6).

This result reveals that a present membrane-bound cytokine-TNFRSF molecule chimeric ligand can make a contribution to improvement of proliferation ability of an immune cell under a cytokine-free culture condition without depending on the length a linker peptide.

TABLE 6

| | Median of Change Rate of Cell Number at Culture |
|---|---|
| Length of Linker | End Point (First Quartile-Third Quartile) |
| 62aa | 46.0(41.3-59.1) |
| 30aa | 37.4(35.2-44.1) |
| 15aa | 23.2(12.8-27.0) |

### Example 6: a Ligand Molecule Linked to a TNFRSF Molecule Activates the TNFRSF Molecule Through a Bond to a Receptor Thereof

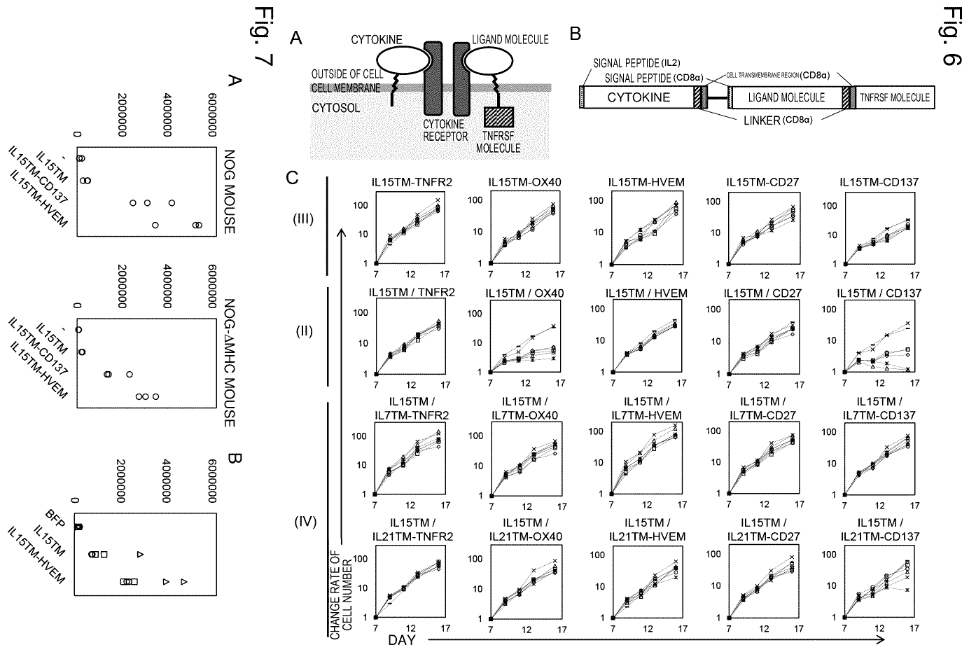

Regarding the expression pattern of the type (II) in which membrane-bound IL15 and a TNFRSF molecule-derived intracellular domain are independently expressed, a method for further improving the cell proliferation was examined. Specifically, the expression pattern of a type (IV) in which membrane-bound IL15, and a membrane-bound ligand molecule linked to a TNFRSF molecule (namely, a “present membrane-bound ligand molecule-TNFRSF molecule chimeric ligand”) are independently expressed was examined (FIG. 6A). In order to produce a T cell expressing a molecule of the type (IV), a gene encoding a polypeptide, in which an IL2-derived signal peptide (SEQ ID NO: 3), IL15 (SEQ ID NO: 22), a CD8α-derived linker peptide (SEQ ID NO: 15), a CD8α-derived transmembrane domain (SEQ ID NO: 16), a spacer (GSG), self-cleaving peptide T2A (SEQ ID NO: 14), an IL2-derived signal peptide (SEQ ID NO: 3), any of two ligand molecules (IL7 [SEQ ID NO: 20] and IL21 [SEQ ID NO: 23]), a CD8α-derived linker peptide (SEQ ID NO: 15), a CD8α-derived transmembrane domain (SEQ ID NO: 16), and an intracellular domain derived from any one of five TNFRSF molecules (TNFR2, OX40, HVEM, CD27, and CD137) (SEQ ID NOs: 5 to 9) (IL15TM/IL7TM-TNFR2, IL15TM/IL7TM-OX40, IL15TM/IL7TM-HVEM, IL15TM/IL7TM-CD27, or IL15TM/IL7TM-CD137; IL15TM/IL21TM-TNFR2, IL15TM/IL21TM-OX40, IL15TM/IL21TM-HVEM, IL15TM/IL21TM-CD27, or IL15TM/IL21TM-CD137), was introduced into a multicloning site of pLVSIN IRES-BFP to produce an expression vector (FIG. 6B). Expression vectors for molecules of the types (II) and (III) used as a comparison control were produced in accordance with the method described above in Example 3. The thus produced expression vectors were used to produce various lentiviruses in accordance with the method described above in [Preparation of Lentivirus]. PBMCs derived from seven donors, and suspended in IL2 medium were respectively added to CD3 Ab/RetroNectin coated plates, and the resultant PBMCs were infected with the produced lentiviruses in accordance with the method described above in [Preparation of Target Gene Expressing T cell], and the resultant was cultured in a basal medium from day 3 of the culture. Besides, a BFP expressing control T cell used as a comparison control was also cultured similarly. In accordance with the method described above in [T Cell Proliferation Test under Cytokine-free Condition], the sampling was performed over time, and the number of BFP positive cells was counted with a flow cytometer. As the test result, a change rate of cell number was evaluated as magnification change based on the number on day 7 of the culture when the proliferation of the BFP expressing control T cell stopped.

As a result, the proliferation efficiency of T cells expressing the molecules of the type (IV) (specifically, combinations of membrane-bound IL15 with intracellular domains derived from the five TNFRSF molecules [TNFR2, OX40, HVEM, CD27, and CD137] linked to membrane-bound IL7 or membrane-bound IL21) was all improved as compared with the molecules of the type (II) corresponding to the type (IV) (specifically, combinations of membrane-bound IL15 with the intracellular domains derived from the five TNFRSF molecule-derived intracellular domains not linked to membrane-bound IL7 or membrane-bound IL21) (FIG. 6C, and Table 7). Membrane-bound IL7 and membrane-bound IL21 did not singly improve the proliferation of a T cell (FIG. 4, and Table 4), and therefore, it is presumed that signaling of the TNFRSF molecule was highly possibly improved through the bond between such a cytokine (ligand molecule) and a receptor thereof. In other words, it is presumed that an extracellular domain linked to a TNFRSF molecule-derived intracellular domain can be replaced with any ligand molecule under a condition in which conventional membrane-bound IL15 is expressed as long as it is a ligand molecule of a receptor present in an immune cell, and a mechanism for activating a downstream TNFRSF molecule through the bond between such a ligand and the receptor can be assumed.

TABLE 7
Change Rate and Improvement Rate of Cell Number
of T Cell Expressing Molecule of Type (IV)

| Expressed | Median of Change Rate of Cell Number at Culture | | Improvement Rate | |
|---|---|---|---|---|
| Molecule | End Point (First Quartile-Third Quartile) | | (Control Group) | |
| IL15TM-TNFR2 | 83.1 | (69.8-93.2) | | |
| IL15TM/TNFR2 | 41.0 | (39.1-44.8) | | |
| IL15TM/IL7TM- | 72.4 | (67.9-104.2) | 100% | (IL15TM/TNFR2) |
| TNFR2 | | | | |
| IL15TM/IL21TM- | 59.3 | (58.0-76.6) | 100% | (IL15TM/TNFR2) |
| TNFR2 | | | | |
| IL15TM-OX40 | 51.4 | (44.9-61.8) | | |
| IL15TM/OX40 | 6.5 | (5.1-20.7) | | |
| IL15TM/IL7TM- | 49.6 | (42.2-58.0) | 100% | (IL15TM/OX40) |
| OX40 | | | | |
| IL15TM/IL21TM- | 45.8 | (38.6-53.0) | 100% | (IL15TM/OX40) |
| OX40 | | | | |
| IL15TM-HVEM | 71.1 | (50.5-74.3) | | |
| IL15TM/HVEM | 29.4 | (28.8-34.7) | | |
| IL15TM/IL7TM- | 79.6 | (68.3-103.0) | 100% | (IL15TM/HVEM) |
| HVEM | | | | |
| IL15TM/IL21TM- | 39.5 | (34.0-42.2) | 57% | (IL15TM/HVEM) |
| HVEM | | | | |
| IL15TM-CD27 | 48.8 | (34.6-56.1) | | |
| IL15TM/CD27 | 25.6 | (24.8-29.2) | | |
| IL15TM/IL7TM- | 52.3 | (47.7-62.7) | 100% | (IL15TM/CD27) |
| CD27 | | | | |
| IL15TM/IL21TM- | 38.3 | (34.2-54.6) | 100% | (IL15TM/CD27) |
| CD27 | | | | |
| IL15TM-CD137 | 19.6 | (18.1-28.4) | | |
| IL15TM/CD137 | 5.2 | (2.4-15.0) | | |
| IL15TM/IL7TM- | 53.4 | (41.1-67.7) | 100% | (IL15TM/CD137) |
| CD137 | | | | |
| IL15TM/IL21TM- | 35.1 | (23.7-52.4) | 86% | (IL15TM/CD137) |

CD137

“Improvement Rate” shown in the table means a rate of the number of donors having, at the end point, a higher change rate of cell number than that of a control group, and having a change rate of cell number of 1 or more.

### Example 7: A T Cell Expressing a Present Membrane-Bound Cytokine-TNFRSF Molecule Chimeric Ligand Remarkably Proliferates Also In Vivo

Since a T cell expressing a present membrane-bound cytokine-TNFRSF molecule chimeric ligand exhibited favorable proliferation in vitro as described in Examples 2 to 6, it was examined whether this proliferation was reproduced also in vivo. When a human T cell is transplanted to a normal animal, it is eliminated by the immunity of the host, and therefore, an NOG mouse, that is, a severely immunodeficient mouse, was used. Besides, in transplantation of a human T cell to an NOG mouse, it is known that graft-versus-host disease (GvHD) is caused because the transplanted cell is activated through a reaction with the host of the mouse, which seems to be advantageous for T cell proliferation. Therefore, an NOG-ΔMHC mouse that was designed to reduce GvHD was also used in examination.

Each of the four T cells used in Example 2 (the BFP expressing control T cell, the IL15TM expressing T cell, the “IL15TM-CD137” expressing T cell, and the “IL15TM-HVEM” expressing T cell) was transplanted in an amount of 1×10⁶ cells to either of two mice (an NOG mouse or an NOG-ΔMHC mouse) in accordance with the method described above in [In Vivo Proliferation Test], and the number of BFP positive cells contained in the spleen after 10 days was counted.

As a result, it was found that when the “IL15TM-CD137” expressing T cell or the “IL15TM-HVEM” expressing T cell was administered to the two mice, the number of BFP positive cells contained in the spleen 10 days after the transplantation was largely increased as compared with the case in which the BFP expressing control T cell or the IL15TM expressing T cell was administered (FIG. 7A).

This result reveals that when an immune cell expressing a present membrane-bound cytokine-TNFRSF molecule chimeric ligand is transplanted to a living body, the immune cell can favorably survive and proliferate in the living body.

Next, in order to confirm that the efficient proliferation, in a living body of an NOG mouse, of a T cell expressing a present membrane-bound cytokine-TNFRSF molecule chimeric ligand does not depend on a donor corresponding to the origin of the T cell, three T cells (a BFP expressing control T cell, an IL15TM expressing T cell, and an “IL15TM-HVEM” expressing T cell) were produced from PBMCs derived from three donors, each of the cells was transplanted in an amount of 2×10⁶ cells to an NOG mouse in accordance with the method described above in [In Vivo Proliferation Test], and the number of BFP positive cells contained in the spleen after 9 days was counted.

As a result, when the “IL15TM-HVEM” expressing T cell was administered to an NOG mouse, the number of BFP positive cells contained in the spleen 9 days after the transplantation, and the cell proliferation efficiency in the living body of the mouse were higher, no matter which donor the cell was derived, as compared with the case in which the BFP expressing control T cell and the IL15TM expressing T cell were administered (FIG. 7B).

This result reveals that an immune cell expressing a present membrane-bound cytokine-TNFRSF molecule chimeric ligand can favorably survive and proliferate in a living body without depending on a donor from which the immune cell is derived.

### Example 8: A CAR-T Cell Expressing a Present Membrane-Bound Cytokine-TNFRSF Molecule Chimeric Ligand Exhibits Favorable Cytotoxic Activity

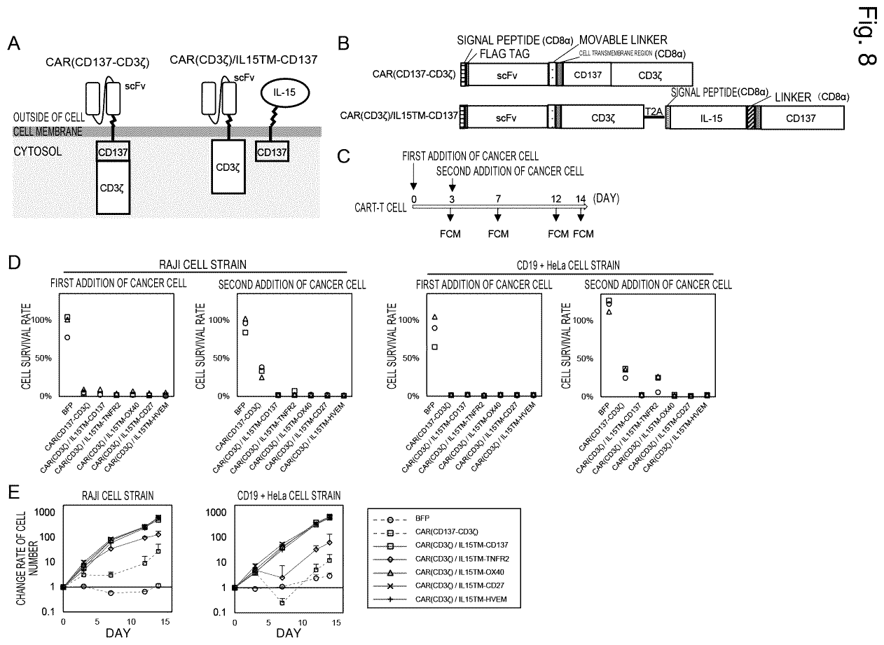

An example of a T cell for treatment expressing a present membrane-bound cytokine-TNFRSF molecule chimeric ligand can be a CAR-T cell. Although those currently used for treatment are mainly what is called second generation CARs, a co-stimulatory molecule (TNFRSF molecule) is already used for transmitting a signal in a present membrane-bound cytokine-TNFRSF molecule chimeric ligand that has the expression pattern of the type (III). Therefore, as a T cell expressing a present membrane-bound cytokine-TNFRSF molecule chimeric ligand, a T cell expressing a CAR not containing a co-stimulatory molecule (CD3ζ), designated as the first generation CAR, was used. First, in order to produce a CAR (CD3ζ-CD137) expressing T cell that is a second generation CAR, a gene encoding a polypeptide (“CD137-CD3ζ” of FIG. 8B), in which a CD8α-derived signal peptide (SEQ ID NO: 4), FLAG tag (SEQ ID NO: 28), a spacer sequence (GSG), an scFv recognizing CD19 (SEQ ID NO: 26), a flexible linker peptide (SEQ ID NO: 27), a CD8α-derived transmembrane domain (SEQ ID NO: 16), a CD137-derived intracellular domain (SEQ ID NO: 9), and a CD3ζ intracellular domain (SEQ ID NO: 24) were successively linked, was introduced into a multicloning site of pLVSIN IRES-BFP to produce an expression vector. Besides, in order to produce a T cell co-expressing a present membrane-bound cytokine-TNFRSF molecule chimeric ligand having the expression pattern of the type (III) and a CAR (CD3ζ), a gene encoding a polypeptide, in which a CD8α-derived signal peptide (SEQ ID NO: 4), FLAG tag sequence (SEQ ID NO: 28), an scFv recognizing CD19 (SEQ ID NO: 26), a flexible linker peptide (SEQ ID NO: 27), a CD8α-derived transmembrane domain (SEQ ID NO: 16), a CD3ζ intracellular domain (SEQ ID NO: 24), a spacer (GSG), self-cleaving peptide T2A (SEQ ID NO: 14), an IL2-derived signal peptide (SEQ ID NO: 3), IL15 (SEQ ID NO: 22), a CD8α-derived linker peptide (SEQ ID NO: 15), a CD8α-derived transmembrane domain (SEQ ID NO: 16), and an intracellular domain derived from any one of five TNFRSF molecules (TNFR2, OX40, HVEM, CD27, and CD137) (SEQ ID NOs: 5 to 9) were successively linked (“CAR (CD3ζ)/IL15TM-TNFR2”, “CAR (CD3ζ)/IL15TM-OX40”, “CAR (CD3ζ)/IL15TM-HVEM”, “CAR (CD3ζ)/IL15TM-CD27”, and “CAR (CD3ζ)IL15TM-CD137”), was introduced into a multicloning site of pLVSIN IRES-BFP to produce an expression vector. The thus produced expression vectors were used to produce lentiviruses in accordance with the method described above in [Preparation of Lentivirus]. PBMCs derived from three donors, and suspended in IL2 medium were respectively added to CD3 Ab/RetroNectin coated plates, and the resultant PBMCs were infected with the produced lentiviruses in accordance with the method described above in [Preparation of Target Gene Expressing T cell]. The resultant was cultured, from day 3 of the culture, in IL2 medium for the “CAR (CD137-CD3ζ)” expressing T cell, and in the basal medium for the T cells co-expressing CAR (CD3ζ), and the five present membrane-bound cytokine-TNFRSF molecule chimeric ligands (“IL15TM-TNFR2”, “IL15TM-OX40”, “IL15TM-HVEM”, “IL15TM-CD27”, and “IL15TM-CD137”). Besides, a BFP expressing control T cell used as a comparison control was prepared with IL2 medium. On day 7 of the culture, the cells were collected, and cytotoxic activity against two cancer cell lines (RAJI cell line, and CD19+HeLa cell line) were evaluated in accordance with the method described above in “Evaluation of Cytotoxic Activity” (FIG. 8C).

As a result, it was found that the five present membrane-bound cytokine-TNFRSF molecule chimeric ligand expressing CAR-T cells exhibited strong cytotoxic activity even in using any of the TNFRSF molecules (FIG. 8D), and efficiently proliferated (FIG. 8E). In particular, the five present membrane-bound cytokine-TNFRSF molecule chimeric ligand expressing CAR-T cells exhibited stronger cytotoxic activity against the cancer cells than the existing second generation CAR-T cell (namely, the “CAR (CD137-CD3ζ)” expressing T cell) in the second addition of the cancer cells, which suggests that these cells have persistent cytotoxic activity against cancer.

### Example 9: A Present Membrane-Bound Cytokine-TNFRSF Molecule Chimeric Ligand Expressing CAR-T Cell Exhibits Favorable Antitumor Activity

Since it was confirmed, in an in vitro system in Example 8, that a present membrane-bound cytokine-TNFRSF molecule chimeric ligand expressing CAR-T cell has stronger cytotoxic activity against cancer than the conventional CAR-T cell, it was evaluated whether this effect was exhibited in vivo. Specifically, two CAR-T cells (the “CAR (CD137-CD3ζ)” expressing T cell and the “CAR (CD3ζ)/IL15TM-OX40” expressing T cell) were used to analyze the antitumor effect in accordance with the method described above in “Evaluation of Antitumor Activity”.

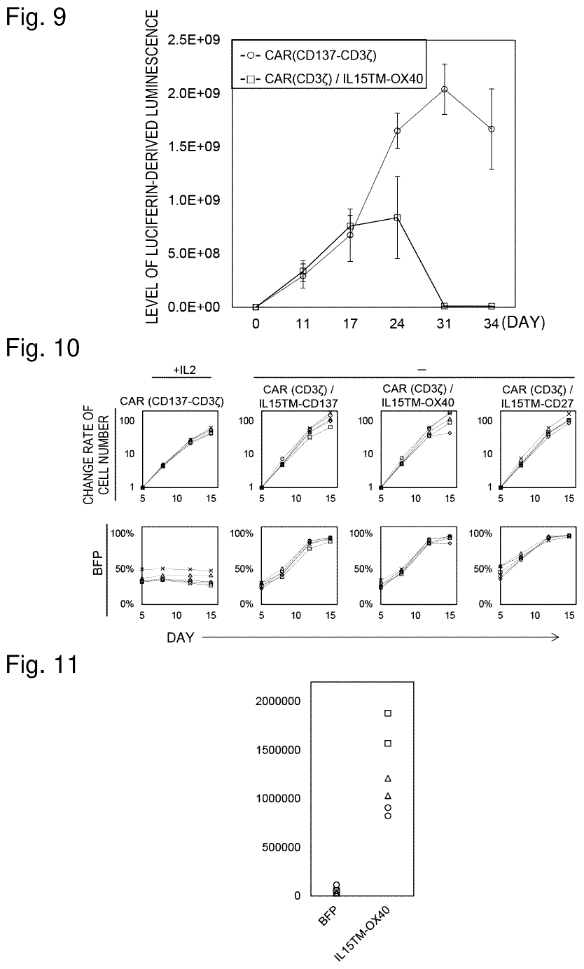

As a result, when the “CAR (CD3ζ)/IL15TM-OX40” expressing T cell that is a present membrane-bound cytokine-TNFRSF molecule chimeric ligand expressing CAR-T cell was transplanted to a tumor-bearing mouse model in which luciferase expressing CD19+HeLa cell had been transplanted, the level of luciferin-derived luminescence was remarkably reduced as compared with the case in which “CAR (CD137-CD3ζ)” expressing T cell that is a conventional CAR-T cell was transplanted (FIG. 9).

This result reveals that a present membrane-bound cytokine-TNFRSF molecule chimeric ligand expressing CAR-T cell has stronger antitumor activity than a conventional CAR-T cell.

### Example 10: A Chimeric Ligand Expressing Cell Exhibits a Concentration Effect

At the time of production of a CAR-T cell for treatment, a ratio of gene expression needs to be at a prescribed level or higher as part of quality control, and if the ratio is lower than a reference value, a production failure that the products cannot be shipped as a therapeutic agent is caused, and treatment cannot be performed in such a case. A present membrane-bound cytokine-TNFRSF molecule chimeric ligand expressing CAR-T cell remarkably proliferates in in vitro culture using a basal medium, but a T cell into which the gene has not been introduced does not proliferate, and therefore, this difference in the proliferation was used to evaluate whether a ratio of gene expressing cells in the whole cell population could be increased.

First, in order to produce a CAR (CD3ζ-CD137) expressing T cell that is the second generation CAR, a gene encoding a polypeptide, in which a CD8α-derived signal peptide (SEQ ID NO: 4), FLAG tag (SEQ ID NO: 28), a spacer (GSG), an scFv recognizing CD19 (SEQ ID NO: 26), a CD28-derived linker peptide (SEQ ID NO: 29), a CD8α-derived transmembrane domain (SEQ ID NO: 16), a CD137-derived intracellular domain (SEQ ID NO: 9), and a CD3ζ intracellular domain (SEQ ID NO: 24) were successively linked, was introduced into a multicloning site of pLVSIN IRES-BFP to produce an expression vector. Besides, in order to produce a present membrane-bound cytokine-TNFRSF molecule chimeric ligand expressing CAR-T cell, a gene encoding a polypeptide, in which a CD8α-derived signal peptide (SEQ ID NO: 4), FLAG tag (SEQ ID NO: 28), a spacer (GSG), an scFv recognizing CD19 (SEQ ID NO: 26), a CD28-derived linker peptide (SEQ ID NO: 29), a CD8α-derived transmembrane domain (SEQ ID NO: 16), a CD3ζ intracellular domain (SEQ ID NO: 24), a spacer (GSG), self-cleaving peptide T2A (SEQ ID NO: 14), an IL2-derived signal peptide (SEQ ID NO: 3), IL15 (SEQ ID NO: 22), a CD8α-derived linker peptide (SEQ ID NO: 15), a CD8α-derived transmembrane domain (SEQ ID NO: 16), and an intracellular domain derived from any one of three TNFRSF molecules (OX40, CD27, and CD137) (SEQ ID NOs: 6, 8, and 9) were successively linked, was introduced into a multicloning site of pLVSIN IRES-BFP to produce an expression vector. PBMCs derived from five donors, and suspended in IL2 medium were respectively added to CD3 Ab/RetroNectin coated plates, and the PBMCs were infected with the produced lentivirus in accordance with the method described above in [Preparation of Target Gene Expressing T Cell]. The present membrane-bound cytokine-TNFRSF molecule chimeric ligand expressing CAR-T cell was cultured in a basal medium from after 3 days, and the CAR (CD3ζ-CD137) expressing T cell was cultured in IL2 medium. In accordance with the method described above in [T Cell Proliferation Test under Cytokine-free Condition], the sampling was performed over time, and the number of BFP positive cells was counted with a flow cytometer. As the test result, a change rate of cell number was evaluated as magnification change based on the number on day 5 of the culture when the proliferation of the BFP expressing control T cell stopped.

As a result, it was revealed that the ratio in the whole cells of the “CAR (CD137-CD3ζ)” expressing T cell detected as the BFP positive cells was about 32% and was not substantially changed even through the culture for 15 days, but the ratios in the whole cells of the CAR-T cells expressing the three present-bound membrane-bound cytokine-TNFRSF molecule chimeric ligands (“IL15TM-OX40”, “IL15TM-CD27”, and “IL15TM-CD137”) detected as the BFP positive cells were increased up to about 96% through the culture for 15 days or more (FIG. 10, and Table 8).

This result reveals that when a cell population containing a present membrane-bound cytokine-TNFRSF molecule chimeric ligand expressing CAR-T cell is cultured in the absence of cytokines (specifically, IL15, IL2, and IL7), the ratio in the cell population of the present membrane-bound cytokine-TNFRSF molecule chimeric ligand expressing CAR-T cell can be increased.

TABLE 8
Effect of Concentrating Present Membrane-bound Cytokine-TNFRSF Molecule Chimeric Ligand
Expressing CAR-T Cell by Present Membrane-bound Cytokine-TNFRSF Molecule Chimeric Ligand

| | Median of Change Rate of Cell Number at Culture | Rate of Gene | |
|---|---|---|---|
| Expressed Molecule | End Point (First Quartile-Third Quartile) | Expressing Cells | |
| CAR (CD137-CD3ζ) | 80.3(66.1-114.4) | 32.0% | (29.7%-41.5%) |
| CAR (CD3ζ)/IL15TM- | 209.5(61.6-249.4) | 94.3% | (92.6%-94.4%) |
| OX40 | | | |
| CAR (CD3ζ)/IL15TM- | 246.5(69.7-280.2) | 96.4% | (94.3%-96.4%) |
| CD27 | | | |
| CAR (CD3ζ)/IL15TM- | 198.0(172.5-199.0) | 97.7% | (97.3%-97.8%) |

CD137

### Example 11: A Present Membrane-Bound Cytokine-TNFRSF Molecule Chimeric Ligand Expressing T Cell can Reduce a Culture Period In Vitro

At the time of production of a CAR-T cell for treatment, a culture period of about 14 days is generally required for obtaining a necessary number of cells. In order not to improve a patient access but to rapidly start the treatment, the culture period is desired to be reduced. A present membrane-bound cytokine-TNFRSF molecule chimeric ligand expressing T cell expresses a cytokine necessary for proliferation by itself, and therefore, it was evaluated whether the T cell could proliferate to reach a necessary number of cells in a living body without culture for several days before administration to the living body.

The expression vector used for producing the “IL15TM-OX40” expressing T cell in Example 2 was used to produce a lentivirus in accordance with the method described above in [Preparation of Lentivirus]. 1×10⁷ PBMCs derived from three donors, and suspended in IL2 medium were respectively added to CD3 Ab/RetroNectin coated plates to infect the PBMCs with the produced lentivirus in accordance with the method described above in [Preparation of Target Gene Expressing T Cell]. Besides, a BFP expressing control T cell used as a comparison control was similarly infected with the PBMC. On the next day (after 16 hours), the resultant cells were collected, and suspended in 200 μL of PBS, and the resultant was transplanted in an NOG mouse in accordance with the method described above in [In Vivo Proliferation Test] to count the number of BFP positive cells contained in the spleen after 9 days. Besides, a part (1 μL) of the administered liquid was separated, seeded in a 96 well plate, and cultured in IL2 medium, and the number of viable transfected T cells (BFP positive cells) was counted in accordance with the method described above in [Flow Cytometry] after 5 days.

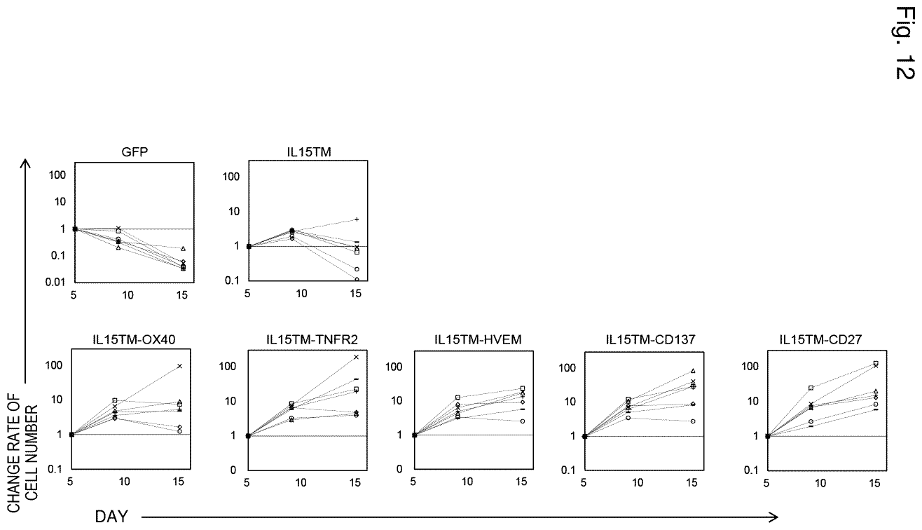

First, it was confirmed that the gene introduction efficiency of the BFP expressing control T cell and that of the “IL15TM-OX40” expressing T cell were equivalent (Table 9). On the other hand, when the “IL15TM-OX40” expressing T cell not precedently cultured was transplanted to an NOG mouse, the number of BFP positive cells contained in the spleen 9 days after the transplantation, and the cell proliferation efficiency in the living body were higher as compared with a case in which the BFP expressing control T cell was transplanted to an NOG mouse (FIG. 11).

This result reveals that there is no need to precedently culture, for more than 10 days, an immune cell expressing a present membrane-bound cytokine-TNFRSF molecule chimeric ligand like a CAR-T cell, and the immune cell can favorably survive and proliferate in a living body by culture for at least 16 hours.

TABLE 9
Gene Transfer Efficiency of Gene Transfected
Cell Used in Animal Experiment

| Donor | BFP | IL15TM-OX40 |
|---|---|---|
| #1 | 50.2% | 47.0% |
| #2 | 32.4% | 31.9% |
| #3 | 43.6% | 35.1% |

### Example 12: A Chimeric Ligand Proliferates an NK Cell

In order to confirm that a present membrane-bound cytokine-TNFRSF molecule chimeric ligand can improve cell proliferation of an immune cell except for a T cell, the effect of improving cell proliferation of an NK cell was evaluated.

In order to produce NK cells respectively expressing five present membrane-bound cytokine-TNFRSF molecule chimeric ligands (“IL15TM-OX40”, “IL15TM-TNFR2”, “IL15TM-HVEM”, “IL15TM-CD137”, and “IL15TM-CD27”), a gene encoding a polypeptide, in which an IL2-derived signal peptide (SEQ ID NO: 3) was added to the N terminal side of IL15 (SEQ ID NO: 22), and a CD8α-derived linker peptide (SEQ ID NO: 15), a CD8α-derived transmembrane domain (SEQ ID NO: 16), and an intracellular domain derived from any one of five TNFRSF molecules (TNFR2, OX40, HVEM, CD27, and CD137) (SEQ ID NOs: 5 to 9) were successively linked to the C terminal side of IL15 (SEQ ID NO: 22), was linked to a gene encoding a spacer (GSG), a self-cleaving peptide T2A (SEQ ID NO: 14), and GFP, and the resultant was inserted downstream of a promoter of pMSGV1 to produce an expression vector. In accordance with the method described above in [Preparation of Retrovirus], a retrovirus was produced. Besides, in order to produce an IL15TM expressing NK cell, a gene encoding a polypeptide in which an IL2-derived signal peptide (SEQ ID NO: 3) was added to the N terminal of IL15TM (SEQ ID NO: 1) was linked to a gene encoding a spacer (GSG), self-cleaving peptide T2A (SEQ ID NO: 14), and GFP, and the resultant was inserted downstream of a promoter of pMSGV1 to produce an expression vector. In accordance with the method described above in [Preparation of Retrovirus], a retrovirus was produced. Besides, in order to produce a GFP expressing control NK cell, a gene encoding a spacer (GSG), self-cleaving peptide T2A (SEQ ID NO: 14), and GFP was inserted downstream of a promoter of pMSGV1 to produce an expression vector. In accordance with the method described above in [Preparation of Retrovirus], a retrovirus was produced. In accordance with the method described above in [Preparation of Target Gene Expressing NK Cell], CD56 positive cells were prepared respectively from seven donors, and were infected with retrovirus produced through addition to a CD16 Ab/RetroNectin coated plate, and the resultant was cultured in NK medium from after 3 days. In accordance with the method described above in [NK Cell Proliferation Test under Cytokine-free Condition], sampling was performed over time to count the number of GFP expressing cells in NK cells (CD3-negative CD56 positive) with a flow cytometer. As the test result, a change rate of cell number was evaluated as magnification change based on the number on day 5 of the culture when the proliferation of GFP expressing control NK cell stopped.

As a result, in all the NK cells expressing the five present membrane-bound cytokine-TNFRSF molecule chimeric ligands (“IL15TM-OX40”, “IL15TM-TNFR2”, “IL15TM-HVEM”, “IL15TM-CD137”, and “IL15TM-CD27”), the change rate of cell number was improved as compared with the IL15TM expressing NK cell in most of the donors, and was improved by 8 to 50 times in terms of the median (FIG. 12, and Table 10).

This result reveals that a present membrane-bound cytokine-TNFRSF molecule chimeric ligand can improve the cell proliferation of the whole immune cells.

TABLE 10
Change Rate and Improvement Rate of Cell Number of Present Membrane-
bound Cytokine-TNFRSF Molecule Chimeric Ligand Expressing NK Cell

| Expressed | Median of Change Rate of Cell Number at Culture | Improvement Rate | |
|---|---|---|---|
| Molecule | End Point (First Quartile-Third Quartile) | (Control Group) | |
| IL15TM | 0.2(0.1-0.4) | | |
| IL15TM-OX40 | 1.7(1.0-5.4) | 86% | (IL15TM) |
| IL15TM-TNFR2 | 4.6(1.3-8.6) | 100% | (IL15TM) |
| IL15TM-HVEM | 3.2(2.4-7.6) | 100% | (IL15TM) |
| IL15TM-CD137 | 10.5(3.5-16.5) | 100% | (IL15TM) |
| IL15TM-CD27 | 4.3(3.1-81.2) | 100% | (IL15TM) |

“Improvement Rate” shown in the table means a rate of the number of donors having, at the end point, a higher change rate of cell number than that of a control group, and having a change rate of cell number of 1 or more.

### INDUSTRIAL APPLICABILITY

The present invention contributes to treatment or prevention of a disease using an immune cell for treatment or prevention.

[Sequence Listing Free Text]

SEQ ID NO: 1: IL15TM (artificial)

| | |
|---|---|
| NWVNVISDLK KIEDLIQSMH IDATLYTESD VHPSCKVTAM KCFLLELQVI SLESGDASIH | 60 |
| DIVENLIILA NNSLSSNGNV TESGCKECEE LEEKNIKEFL QSFVHIVQMF INTSSIMYFS | 120 |
| HFVPVFLPAK PTTTPAPRPP TPAPTIASQP LSLRPEACRP AAGGAVHTRG LDFACDIYIW | 180 |
| APLAGTCGVL LLSLVITLYC | 200 |

SEQ ID NO: 2: IL15-IL15Ra (Artificial)

| | |
|---|---|
| NWVNVISDLK KIEDLIQSMH IDATLYTESD VHPSCKVTAM KCFLLELQVI SLESGDASIH | 60 |
| DTVENLIILA NNSLSSNGNV TESGCKECEE LEEKNIKEFL QSFVHIVQMF INTSSGGGSG | 120 |
| GGGSGGGGSG GGGSGGGSLQ ITCPPPMSVE HADIWVKSYS LYSRERYICN SGFKRKAGTS | 180 |
| SLTECVLNKA INVAHWTTPS LKCIRDPALV HQRPAPPSTV TTAGVTPQPE SLSPSGKEPA | 240 |
| ASSPSSNNTA ATTAAIVPGS QLMPSKSPST GTTEISSHES SHGTPSQTTA KNWELTASAS | 300 |
| HQPPGVYPQG HSDTTVAIST STVLLCGLSA VSLLACYLKS RQTPPLASVE MEAMEALPVT | 360 |
| WGISSRDEDL ENCSHHL | 377 |

SEQ ID NO: 3: IL-2 derived signal peptide (Homo sapiens)

| | |
|---|---|
| MYRMQLLSCI ALSLALVINS | 20 |

SEQ ID NO: 4: CD80 derived signal peptide (Homo sapiens)

| | |
|---|---|
| MALPVTALLL PLALLLHAAR P | 21 |

SEQ ID NO: 5: Intracellular domain of TNFR2 (Homo sapiens)

| | |
|---|---|
| KKKPLCLQRE AKVPHLPADK ARGTQGPEQQ HLLITAPSSS SSSLESSASA LDRRAPTRNQ | 60 |
| PQAPGVEASG AGEARASTGS SDSSPGGHGT QVNVTCIVNV CSSSDHSSQC SSQASSIMGD | 120 |
| TDSSPSESPK DEQVPFSKEE CAFRSQLETP ETLLGSTEEK PLPLGVPDAG MKPS | 174 |

SEQ ID NO: 6: Intracellular domain of Ox40 (Homo sapiens)

| | |
|---|---|
| RRDQRLPPDA HKPPGGGSFR TPIQEEQADA HSTLAKI | 37 |

SEQ ID NO: 7: Intracellular domain of HVEM (Homo sapiens)

| | |
|---|---|
| CVKRRKPRGD VVKVIVSVQR KRQEAEGEAT VIEALQAPPD VTTVAVEETI PSFTGRSPNH | 60 |

SEQ ID NO: 8: Intracellular domain of CD27 (Homo sapiens)

| | |
|---|---|
| QRRKYRSNKG ESPVEPAEPC RYSCPREEEG STIPIQEDYR KPEPACSP | 48 |

SEQ ID NO: 9: Intracellular domain of CD137 (Homo sapiens)

| | |
|---|---|
| KRGRKKLLYI FKQPFMRPVQ TTQEEDGCSC RFPEEEEGGC EL | 42 |

SEQ ID NO: 10: Intracellular domain of CD30 (Homo sapiens)

| | |
|---|---|
| CHRRACRKRI RQKLHLCYPV QTSQPKLELV DSRPRRSSTQ LRSGASVTEP VAEERGLMSQ | 60 |
| PLMETCHSVG AAYLESLPLQ DASPAGGPSS PRDLPEPRVS TEHTNNKIEK IYIMKADTVI | 120 |
| VGTVKAELPE GRGLAGPAEP ELEEELEADH TPHYPEQETE PPLGSCSDVM LSVEEEGKED | 180 |
| PLPTAASGK | 189 |

SEQ ID NO: 11: Intracellular domain of DR3 (Homo sapiens)

| | |
|---|---|
| RHCWPHKPLV TADEAGMEAL TPPPATHLSP LDSAHTLLAP PDSSEKICTV QLVGNSWTPG | 60 |
| YPETQEALCP QVTWSWDQLP SRALGPAAAP TLSPESPAGS PAMMLQPGPQ LYDVMDAVPA | 120 |
| RRWKEFVRTL GLREAEIEAV EVEIGRFRDQ QYEMLKRWRQ QQPAGLGAVY AALERMGLDG | 180 |
| CVEDLRSRLQ RGP | 193 |

SEQ ID NO: 12: Intracellular domain of CD28 (Homo sapiens)

| | |
|---|---|
| RSKRSRLLHS DYMNMTPRRP GPTRKHYQPY APPRDFAAYR S | 41 |

SEQ ID NO: 13: Intracellular domain of ICOS (Homo sapiens)

| | |
|---|---|
| TKKKYSSSVH DPNGEYMFMR AVNTAKKSRL TDVTL | 35 |

SEQ ID NO: 14: self-cleaving peptide T2A (Thosea asigna virus)

| | |
|---|---|
| EGRGSLLTCG DVEENPGP | 18 |

SEQ ID NO: 15: CD8α derived linker peptide (Homo sapiens)

| | |
|---|---|
| SIMYFSHFVP VFLPAKPTTT PAPRPPTPAP TIASQPLSLR PEACRPAAGG AVHTRGLDFA | 60 |
| CD | 62 |

SEQ ID NO: 16: CD8α derived transmembrane domain (Homo sapiens)

| | |
|---|---|
| IYIWAPLAGT CGVLLLSLVI TLYC | 24 |

SEQ ID NO: 17: IL2 (Homo sapiens)

| | |
|---|---|
| APTSSSTKKT QLQLEHLLLD LQMILNGINN YKNPKLTRML TFKFYMPKKA TELKHLQCLE | 60 |
| EELKPLEEVL NLAQSKNFHL RPRDLISNIN VIVLELKGSE TTFMCEYADE TATIVEFLNR | 120 |
| WITFCQSIIS TLT | 133 |

SEQ ID NO: 18: IL4 (Homo sapiens)

| | |
|---|---|
| HKCDITLQEI IKTLNSLTEQ KTLCTELTVT DIFAASKNTT EKETFCRAAT VLRQFYSHHE | 60 |
| KDTRCLGATA QQFHRHKQLI RFLKRLDRNL WGLAGLNSCP VKEANQSTLE NFLERLKTIM | 120 |
| REKYSKCSS | 129 |

SEQ ID NO: 19: IL6 (Homo sapiens)

| | |
|---|---|
| PVPPGEDSKD VAAPHRQPLT SSERIDKQIR YILDGISALR KETCNKSNMC ESSKEALAEN | 60 |
| NLNLPKMAEK DGCFQSGFNE ETCLVKIITG LLEFEVYLEY LQNRFESSEE QARAVQMSTK | 120 |
| VLIQFLQKKA KNLDAITTPD PTTNASLLTK LQAQNQWLQD MTTHLILRSF KEFLQSSLRA | 180 |
| LRQM | 184 |

SEQ ID NO: 20: IL7 (Homo sapiens)

| | |
|---|---|
| DCDIEGKDGK QYESVLMVSI DQLLDSMKEI GSNCLNNEFN FFKRHICDAN KEGMFLFRAA | 60 |
| RKLRQFLKMN STGDFDLHLL KVSEGTTILL NCTGQVKGRK PAALGEAQPT KSLEENKSLK | 120 |
| EQKKLNDLCF LKRLLQEIKT CWNKILMGTK EH | 152 |

SEQ ID NO: 21: IL9 (Homo sapiens)

| | |
|---|---|
| QGCPTLAGIL DINFLINKMQ EDPASKCHCS ANVTSCLCLG IPSDNCTRPC FSERLSQMTN | 60 |
| TTMQTRYPLI FSRVKKSVEV LKNNKCPYFS CEQPCNQTTA GNALTFLKSL LEIFQKEKMR | 120 |
| GMRGKI | 126 |

SEQ ID NO: 22: IL15 (Homo sapiens)

| | |
|---|---|
| NWVNVISDLK KIEDLIQSMH IDATLYTESD VHPSCKVTAM KCFLLELQVI SLESGDASIH | 60 |
| DTVENLIILA NNSLSSNGNV TESGCKECEE LEEKNIKEFL QSFVHIVQMF INTS | 114 |

SEQ ID NO: 23: IL21 (Homo sapiens)

| | |
|---|---|
| HKSSSQGQDR HMIRMRQLID IVDQLKNYVN DLVPEFLPAP EDVETNCEWS AFSCFQKAQL | 60 |
| KSANTGNNER IINVSIKKLK RKPPSTNAGR RQKHRLTCPS CDSYEKKPPK EFLERFKSLL | 120 |
| QKMIHQHLSS RTHGSEDS | 138 |

SEQ ID NO: 24: Intracellular domain of CD3ζ (Homo sapiens)

| | |
|---|---|
| RVKFSRSADA PAYQQGQNQL YNELNLGRRE EYDVLDKRRG RDPEMGGKPR RKNPQEGLYN | 60 |
| ELQKDKMAEA YSEIGMKGER RRGKGHDGLY QGLSTATKDT YDALHMQALP PR | 112 |

SEQ ID NO: 25: Extracellular domain and transmembrane domain of CD19
(Homo sapiens)

| | |
|---|---|
| PEEPLVVKVE EGDNAVLQCL KGTSDGPTQQ LTWSRESPLK PFLKLSLGLP GLGIHMRPLA | 60 |
| IWLFIFNVSQ QMGGFYLCQP GPPSEKAWQP GWTVNVEGSG ELFRWNVSDL GGLGCGLKNR | 120 |
| SSEGPSSPSG KLMSPKLYVW AKDRPEIWEG EPPCLPPRDS LNQSLSQDLT MAPGSTLWLS | 180 |
| CGVPPDSVSR GPLSWTHVHP KGPKSLLSLE LKDDRPARDM WVMETGLLLP RATAQDAGKY | 240 |
| YCHRGNLIMS FHLEITARPV LWHWLLRTGG WKVSAVTLAY LIFCLCSLVG ILHL | 294 |

SEQ ID NO: 26: Anti-CD19 scFv (Artificial)

| | |
|---|---|
| DIQMTQTTSS LSASLGDRVT ISCRASQDIS KYLNWYQQKP DGTVKLLIYH TSRLHSGVPS | 60 |
| RFSGSGSGTD YSLTISNLEQ EDIATYFCQQ GNTLPYTFGG GTKLEITGGG GSGGGGSGGG | 120 |
| GSEVKLQESG PGLVAPSQSL SVTCTVSGVS LPDYGVSWIR QPPRKGLEWL GVIWGSETTY | 180 |
| YNSALKSRLT IIKDNSKSQV FLKMNSLQTD DTAIYYCAKH YYYGGSYAMD YWGQGTSVTV | 240 |
| SS | 242 |

SEQ ID NO: 27: Flexible linker peptide (Artificial)

| | |
|---|---|
| GGGGSGGGGS GGGGS | 15 |

SEQ ID NO: 28: FLAG tag (Artificial)

| | |
|---|---|
| DYKDDDDK | 8 |

SEQ ID NO: 29: CD28 derived linker peptide (Homo sapiens)

| | |
|---|---|
| IEVMYPPPYL DNEKSNGTII HVKGKHLCPS PLFPGPSKP | 39 |

SEQ ID NO: 30: CD28 derived transmembrane domain (Homo sapiens)

| | |
|---|---|
| FWVLVVVGGV LACYSLLVTV AFIIFWVRSK | 30 |

SEQ ID NO: 31: 30 aa of CD8α derived linker peptide (Homo sapiens)

| | |
|---|---|
| ASQPLSLRPE ACRPAAGGAV HTRGLDFACD | 30 |

SEQ ID NO: 32: 15 aa of CD8α derived linker peptide (Homo sapiens)

| | |
|---|---|
| AGGAVHTRGL DFACD | 15 |

SEQ ID NO: 33: IL15 (Mus musculus)

| | |
|---|---|
| NWIDVRYDLE KIESLIQSIH IDTTLYTDSD FHPSCKVTAM NCFLLELQVI LHEYSNMTLN | 60 |
| ETVRNVLYLA NSTLSSNKNV AESGCKECEE LEEKTFTEFL QSFIRIVQMF INTS | 114 |

SEQ ID NO: 34: IL15 (Rattus norvegicus)

| | |
|---|---|
| MNWIDVRYDL EKIESLIQFI HIDTTLYTDS DFHPSCKVTA MNCFLLELQV ILHEYSNMTL | 60 |
| NETVRNVLYL ANSTLSSNKN VIESGCKECE ELEERNFTEF LQSFIHIVQM FINTS | 115 |

SEQ ID NO: 35: IL2 (Mus musculus)

| | |
|---|---|
| APTSSSTSSS TAEAQQQQQQ QQQQQQHLEQ LLMDLQELLS RMENYRNLKL PRMLTFKFYL | 60 |
| PKQATELKDL QCLEDELGPL RHVLDLTQSK SFQLEDAENF ISNIRVTVVK LKGSDNTFEC | 120 |
| QFDDESATVV DFLRRWIAFC QSIISTSPQ | 149 |

SEQ ID NO: 36: IL2 (Rattus norvegicus)

| | |
|---|---|
| APTSSPAKET QQHLEQLLLD LQVLLRGIDN YKNLKLPMML TFKFYLPKQA TELKHLQCLE | 60 |
| NELGALQRVL DLTQSKSFHL EDAGNFISNI RVTVVKLKGS ENKFECQFDD EPATVVEFLR | 120 |
| RWIAICQSII STMTQ | 135 |

SEQ ID NO: 37: IL7 (Mus musculus)

| | |
|---|---|
| ECHIKDKEGK AYESVLMISI DELDKMTGTD SNCPNNEPNF FRKHVCDDTK EAAFLNRAAR | 60 |
| KLKQFLKMNI SEEFNVHLLT VSQGTQTLVN CTSKEEKNVK EQKKNDACFL KRLLREIKTC | 120 |
| WNKILKGSI | 129 |

SEQ ID NO: 38: IL7 (Rattus norvegicus)

| | |
|---|---|
| DCHIKDKDGK AFGSVLMISI NQLDKMTGTD SDCPNNEPNF FKKHLCDDTK EAAFLNRAAR | 60 |
| KLRQFLKMNI SEEFNDHLLR VSDGTQTLVN CTSKEEKTIK EQKKNDPCFL KRLLREIKTC | 120 |
| WNKILKGSI | 129 |

SEQ ID NO: 39: Intracellular domain of TNFR2 (Mus musculus)

| | |
|---|---|
| KKKPSCLQRD AKVPHVPDEK SQDAVGLEQQ HLLTTAPSSS SSSLESSASA GDRRAPPGGH | 60 |
| PQARVMAEAQ GFQEARASSR ISDSSHGSHG THVNVTCIVN VCSSSDHSSQ CSSQASATVG | 120 |
| DPDAKPSASP KDEQVPFSQE ECPSQSPCET TETLQSHEKP LPLGVPDMGM KPSQAGWFDQ | 180 |
| IAVKVA | 186 |

SEQ ID NO: 40: Intracellular domain of INFR2 (Rattus norvegicus)

| | |
|---|---|
| KKKPSCLQRE TMVPHLPDDK SQDAIGLEQQ HLLTTAPSSS SSSLESSASA GDRRAPPGGH | 60 |
| PQARVTAEAQ GSQEACAGSR SSDSSHGSHG THVNVTCIVN VCSSSDHSSQ CSSQASTTVG | 120 |
| DPDANPSGSP KDEQVPFSQE ECPSQSQWET TETLQNHDKP FPLGVPDVGM KPNQPGWYDQ | 180 |
| IAVKVP | 186 |

SEQ ID NO: 41: Intracellular domain of Ox40 (Mus musculus)

| | |
|---|---|
| RKAWRLPNTP KPCWGNSFRT PIQEEHTDAH FTLAKI | 36 |

SEQ ID NO: 42: Intracellular domain of Ox40 (Rattus norvegicus)

| | |
|---|---|
| RKAWRSPNTP KPCWGNSFRT PIQEEQTDTH FTLAKI | 36 |

SEQ ID NO: 43: Intracellular domain of HVEM (Mus musculus)

| | |
|---|---|
| TRRHLHTSSV AKELEPFQEQ QENTIRFPVT EVGFAETEEE TASN | 44 |

SEQ ID NO: 44: Intracellular domain of HVEM (Rattus norvegicus)

| | |
|---|---|
| RKQRQRHTSI VASELEAFQQ EQQEDAIRFP VIEVGPSVTE EEAAFNCMNS G | 51 |

SEQ ID NO: 45: Intracellular domain of CD27 (Mus musculus)

| | |
|---|---|
| QRRNHGPNED RQAVPEEPCP YSCPREEEGS AIPIQEDYRK PEPAFYP | 47 |

SEQ ID NO: 46: Intracellular domain of CD27 (Rattus norvegicus)

| | |
|---|---|
| HQRRNHGPNE DSQAVPEELC PYSCPREEEG SVIPIQEDYR KPEPASYP | 48 |

SEQ ID NO: 47: Intracellular domain of CD137 (Mus musculus)

| | |
|---|---|
| SVLKWIRKKF PHIFKQPFKK TTGAAQEEDA CSCRCPQEEE GGGGGYEL | 48 |

SEQ ID NO: 48: Intracellular domain of CD137 (Rattus norvegicus)

| | |
|---|---|
| SVPKWLRKKF PHIFKQPFKK AVRTAQEEDA CSCRFPEEEE GGGGSYEL | 48 |

SEQ ID NO: 49: IL15 coding region (Homo sapiens)

| | |
|---|---|
| atgagaattt cgaaaccaca tttgagaagt atttccatcc agtgctactt gtgtttactt | 60 |
| ctaaacagtc attttctaac tgaagctggc attcatgtct tcattttggg ctgtttcagt | 120 |
| gcagggcttc ctaaaacaga agccaactgg gtgaatgtaa taagtgattt gaaaaaaatt | 180 |
| gaagatctta ttcaatctat gcatattgat gctactttat atacggaaag tgatgttcac | 240 |
| cccagttgca aagtaacagc aatgaagtgc tttctcttgg agttacaagt tatttcactt | 300 |
| gagtccggag atgcaagtat tcatgataca gtagaaaatc tgatcatcct agcaaacaac | 360 |
| agtttgtctt ctaatgggaa tgtaacagaa tctggatgca aagaatgtga ggaactggag | 420 |
| gaaaaaaata ttaaagaatt tttgcagagt tttgtacata ttgtccaaat gttcatcaac | 480 |
| acttcttga | 489 |

SEQ ID NO: 50: TNFR2 coding region (Homo sapiens)

| | |
|---|---|
| atggcgcccg tcgccgtctg ggccgcgctg gccgtcggac tggagctctg ggctgcggcg | 60 |
| cacgccttgc ccgcccaggt ggcatttaca ccctacgccc cggagcccgg gagcacatgc | 120 |
| cggctcagag aatactatga ccagacagct cagatgtgct gcagcaaatg ctcgccgggc | 180 |
| caacatgcaa aagtcttctg taccaagacc tcggacaccg tgtgtgactc ctgtgaggac | 240 |
| agcacataca cccagctctg gaactgggtt cccgagtgct tgagctgtgg ctcccgctgt | 300 |
| agctctgacc aggtggaaac tcaagcctgc actcgggaac agaaccgcat ctgcacctgc | 360 |
| aggcccggct ggtactgcgc gctgagcaag caggaggggt gccggctgtg cgcgccgctg | 420 |
| cgcaagtgcc gcccgggctt cggcgtggcc agaccaggaa ctgaaacatc agacgtggtg | 480 |
| tgcaagccct gtgccccggg gacgttctcc aacacgactt catccacgga tatttgcagg | 540 |
| ccccaccaga tctgtaacgt ggtggccatc cctgggaatg caagcatgga tgcagtctgc | 600 |
| acgtccacgt cccccacccg gagtatggcc ccaggggcag tacacttacc ccagccagtg | 660 |
| tccacacgat cccaacacac gcagccaact ccagaaccca gcactgctcc aagcacctcc | 720 |
| ttcctgctcc caatgggccc cagcccccca gctgaaggga gcactggcga cttcgctctt | 780 |
| ccagttggac tgattgtggg tgtgacagcc ttgggtctac taataatagg agtggtgaac | 840 |
| tgtgtcatca tgacccaggt gaaaaagaag cccttgtgcc tgcagagaga agccaaggtg | 900 |
| cctcacttgc ctgccgataa ggcccggggt acacagggcc ccgagcagca gcacctgctg | 960 |
| atcacagcgc cgagctccag cagcagctcc ctggagagct cggccagtgc gttggacaga | 1020 |
| agggcgccca ctcggaacca gccacaggca ccaggcgtgg aggccagtgg ggccggggag | 1080 |
| gcccgggcca gcaccgggag ctcagattct tcccctggtg gccatgggac ccaggtcaat | 1140 |
| gtcacctgca tcgtgaacgt ctgtagcagc tctgaccaca gctcacagtg ctcctcccaa | 1200 |
| gccagctcca caatgggaga cacagattcc agcccctcgg agtccccgaa ggacgagcag | 1260 |
| gtccccttct ccaaggagga atgtgccttt cggtcacagc tggagacgcc agagaccctg | 1320 |
| ctggggagca ccgaagagaa gcccctgccc cttggagtgc ctgatgctgg gatgaagccc | 1380 |
| agttaa | 1386 |

SEQ ID NO: 51: CD8α coding region (Homo sapiens)

| | |
|---|---|
| atggccttac cagtgaccgc cttgctcctg ccgctggcct tgctgctcca cgccgccagg | 60 |
| ccgagccagt tccgggtgtc gccgctggat cggacctgga acctgggcga gacagtggag | 120 |
| ctgaagtgcc aggtgctgct gtccaacccg acgtcgggct gctcgtggct cttccagccg | 180 |
| cgcggcgccg ccgccagtcc caccttcctc ctatacctct cccaaaacaa gcccaaggcg | 240 |
| gccgaggggc tggacaccca gcggttctcg ggcaagaggt tgggggacac cttcgtcctc | 300 |
| accctgagcg acttccgccg agagaacgag ggctactatt tctgctcggc cctgagcaac | 360 |
| tccatcatgt acttcagcca cttcgtgccg gtcttcctgc cagcgaagcc caccacgacg | 420 |
| ccagcgccgc gaccaccaac accggcgccc accatcgcgt cgcagcccct gtccctgcgc | 480 |
| ccagaggcgt gccggccagc ggcggggggc gcagtgcaca cgagggggct ggacttcgcc | 540 |
| tgtgatatct acatctgggc gcccttggcc gggacttgtg gggtccttct cctgtcactg | 600 |
| gttatcaccc tttactgcaa ccacaggaac cgaagacgtg tttgcaaatg tccccggcct | 660 |
| gtggtcaaat cgggagacaa gcccagcctt tcggcgagat acgtctaa | 708 |

SEQ ID NO: 52: IL15TM-Ox40 [linker and TM is derived from CD8α] (Artificial)

| | |
|---|---|
| MYRMQLLSCI ALSLALVINS NWVNVISDLK KIEDLIQSMH IDATLYTESD VHPSCKVTAM | 60 |
| KCFLLELQVI SLESGDASIH DTVENLIILA NNSLSSNGNV TESGCKECEE LEEKNIKEFL | 120 |
| QSFVHIVQMF INTSSIMYFS HFVPVFLPAK PTTTPAPRPP TPAPTIASQP LSLRPEACRP | 180 |
| AAGGAVHTRG LDFACDIYIW APLAGTCGVL LLSLVITLYC RRDQRLPPDA HKPPGGGSFR | 240 |
| TPIQEEQADA HSTLAKI | 257 |

(IL2-derived signal peptide: amino acids 1 to 20, IL15 region: amino acids 21 to 134, CD8α-derived linker peptide: amino acids 135 to 196, CD8α-derived transmembrane domain: amino acids 197 to 220, intracellular domain of OX40: amino acids 221 to 257)

| SEQ ID NO: 53: IL15TM-CD137 linker and TM is derived from CD8α] (Artificial) | |
|---|---|
| MYRMQLLSCI ALSLALVINS NWVNVISDLK KIEDLIQSMH IDATLYTESD VHPSCKVTAM | 60 |
| KCFLLELQVI SLESGDASIH DTVENLIILA NNSLSSNGNV TESGCKECEE LEEKNIKEFL | 120 |
| QSFVHIVQMF INTSSIMYFS HFVPVFLPAK PTTTPAPRPP TPAPTIASQP LSLRPEACRP | 180 |
| AAGGAVHTRG LDFACDIYIW APLAGTCGVL LLSLVITLYC KRGRKKLLYI FKQPFMRPVQ | 240 |
| TTQEEDGCSC RFPEEEEGGC EL | 262 |

(IL2 derived signal peptide: 1 to 20, IL15 region: 21 to 134, CD8α derived linker peptide: 135 to 196, CD8α derived transmembrane domain: 197 to 220, Intracellular domain of CD137: 221 to 262)

| SEQ ID NO: 54: IL7TM-Ox40 [linker and TM is derived from CD8] (Artificial) | |
|---|---|
| MYRMOLLSCI ALSLALVINS DCDIEGKDGK QYESVLMVSI DOLLDSMKEI GSNCLNNEFN | 60 |
| FFKRHICDAN KEGMFLFRAA RKLROFLKMN STGDFDLHLL KVSEGTTILL NCTGQVKGRK | 120 |
| PAALGEAQPT KSLEENKSLK EQKKLNDLCF LKRLLQEIKT CWNKILMGTK EHSIMYFSHF | 180 |
| VPVFLPAKPT TTPAPRPPTP APTIASQPLS LRPEACRPAA GGAVHTRGLD FACDIYIWAP | 240 |
| LAGTCGVLLL SLVITLYCRR DORLPPDAHK PPGGGSFRTP IQEEQADAHS TLAKI | 295 |

(IL2 derived signal peptide: 1 to 20, IL7 region: 21 to 172, CD8α derived linker peptide: 173 to 234, CD8α derived transmembrane domain: 235 to 258, Intracellular domain of OX40: 259 to 295)

| SEQ ID NO: 55: IL7TM-CD137 [linker and TM is derived from CD8α] (Artificial) | |
|---|---|
| MYRMQLLSCI ALSLALVINS DCDIEGKDGK QYESVLMVSI DOLLDSMKEI GSNCLNNEFN | 60 |
| FFKRHICDAN KEGMFLFRAA RKLROFLKMN STGDFDLHLL KVSEGTTILL NCTGQVKGRK | 120 |
| PAALGEAQPT KSLEENKSLK EQKKLNDLCF LKRLLQEIKT CWNKILMGTK EHSIMYFSHF | 180 |
| VPVFLPAKPT TTPAPRPPTP APTIASQPLS LRPEACRPAA GGAVHTRGLD FACDIYIWAP | 240 |
| LAGTCGVLLL SLVITLYCKR GRKKLLYIFK QPFMRPVQTT QEEDGCSCRE PEEEEGGCEL | 300 |

(IL2 derived signal peptide: 1 to 20, IL7 region: 21 to 172, CD8α derived linker peptide: 173 to 234, CD806 derived transmembrane domain: 235 to 258, Intracellular domain of CD137: 259 to 300)

| SEQ ID NO: 56: Neo-2/15 (Artificial) | |
|---|---|
| GSHMPKKKIQ LHAEHALYDA LMILNIVKTN SPPAEEKLED YAFNFELILE EIARLFESGD | 60 |
| QKDEAEKAKR MKEWMKRIKT TASEDEQEEM ANAIITILOS WIFS | 104 |

1. A polynucleotide comprising:
an extracellular domain encoding region encoding an extracellular domain containing an amino acid sequence derived from a ligand protein that binds to a receptor of IL15, IL2, or IL7, wherein the binding of the ligand protein to the receptor on an immune cell transmits a signal similar to a binding signal of the cytokine into the immune cell via the receptor; and
an intracellular domain encoding region encoding an intracellular domain containing a region derived from an intracellular domain of a TNF receptor superfamily (TNFRSF) molecule, but not containing an ITAM (Immunoreceptor Tyrosine-based Activation Motif) intracellular signaling region,
wherein (a) the polynucleotide is designed as an extracellular domain-intracellular domain-containing molecule gene that enables to express, on an immune cell, a polypeptide containing both of the extracellular domain and the intracellular domain wherein the extracellular domain and the intracellular domain are linked to each other via a transmembrane domain, or
(b) the polynucleotide is designed as an extracellular domain-containing molecule gene and an intracellular domain-containing molecule gene that enable to express, on the immune cell, a combination of a first polypeptide containing the extracellular domain and a second polypeptide containing the intracellular domain wherein the extracellular domain and the intracellular domain are linked to a first cell membrane binding domain and a second cell membrane binding domain, respectively.

2. The polynucleotide according to claim 1, wherein the IL15, IL2, or IL7, and the TNFRSF molecule are derived from a human, a mouse, or a rat.

3. The polynucleotide according to claim 1, wherein the extracellular domain containing an amino acid sequence derived from the ligand protein that binds to an IL15 receptor contains an amino acid sequence having at least 80% sequence identity with an amino acid sequence shown in SEQ ID NO: 22, and retains activity of binding to the IL15 receptor; the extracellular domain containing an amino acid sequence derived from the ligand protein that binds to an IL2 receptor contains an amino acid sequence having at least 80% sequence identity with an amino acid sequence shown in SEQ ID NO: 17, and retains activity of binding to the IL2 receptor; and the extracellular domain containing an amino acid sequence derived from the ligand protein that binds to an IL7 receptor contains an amino acid sequence having at least 80% sequence identity with an amino acid sequence shown in SEQ ID NO: 20, and retains activity of binding to the IL7 receptor.

4. The polynucleotide according to claim 1, wherein the TNFRSF molecule is TNFR2, OX40, HVEM, CD27, or CD137.

5. The polynucleotide according to claim 1, wherein the immune cell is a T cell or a natural killer cell.

6. The polypeptide according to claim 1, wherein the transmembrane domain is a CD8α-derived transmembrane domain, or a CD28-derived transmembrane domain.

7. The polynucleotide according to claim 1 wherein the extracellular domain and the transmembrane domain are linked to each other via a linker peptide.

8. The polynucleotide according to claim 7, wherein the linker peptide is a CD8α-derived linker peptide, or a CD28-derived linker peptide.

9-10. (canceled)

11. The polynucleotide according to claim 1, wherein the first polypeptide and the second polypeptide are linked to each other via a self-cleaving peptide.

12. (canceled)

13. The polynucleotide according to claim 1, wherein the second polypeptide has an extracellular domain containing an amino acid sequence derived from a ligand molecule of a receptor endogenously present in an immune cell into which the polynucleotide is to be genetically introduced.

14. The polynucleotide according to claim 13, wherein the ligand molecule is IL7 or IL21.

15. The polynucleotide according to claim 1, further encoding a chimeric antigen receptor (CAR) containing a single chain antibody, a transmembrane domain, and an ITAM intracellular signaling region.

16. A vector comprising a promoter, and the polynucleotide according to claim 1, operably linked downstream of the promoter.

17. An immune cell into which the vector according to claim 16 has been introduced.

18. A pharmaceutical composition comprising the immune cell according to claim 17.

19. The pharmaceutical composition according to claim 18, wherein the pharmaceutical composition is arranged to be administered to a subject within 10 days after genetically introducing the vector to the immune cell.

20. A polypeptide comprising:
an intracellular domain containing an amino acid sequence derived from a ligand protein that binds to a receptor of IL15, IL2, or IL7, wherein the binding of the ligand protein to the receptor on an immune cell transmits a signal similar to a binding signal of the cytokine into the immune cell via the receptor;
a transmembrane domain; and
an intracellular domain containing a region derived from an intracellular domain of a TNF receptor superfamily (TNFRSF) molecule, but not containing an ITAM (Immunoreceptor Tyrosine-based Activation Motif) intracellular signaling region.

21-33. (canceled)

34. An immune cell which the polypeptide according to claim 20 is expressed on its cell-membrane.

35-36. (canceled)

37. A method for preparing a cell population comprising an immune cell, the method comprising the following steps (A) and (B) of:
(A) introducing the vector according to claim 16 to an immune cell; and
(B) culturing the immune cell for 5 days or more after the gene introduction.

38. The method according to claim 37, wherein the immune cell resulting from the gene introduction is cultured in the step (B) in the absence of a ligand protein that binds to a receptor of IL15, IL2, or IL7, wherein the binding of the ligand protein to the receptor on an immune cell transmits a signal similar to a binding signal of the cytokine into the immune cell via the receptor.


SEQUENCE LISTING (56 sequences)

SEQ ID NO: 1 - protein, 200 aa, synthetic construct
  REGION 1..200: note = IL15TM
  REGION 1..200: note = MISC_FEATURE - Inventor; YOSHIKIYO, Kazunori; SUDA, Koji; KARASAWA, Hiroshi; YO SHIDA, Sayaka; TAGAYA, Hiroaki
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSIMYFSHFVPVFLPAKPTTTPAPRPPTPAPTIASQPLSLRPEACRPAAGGAVHTRGLDFACDIYIWAPLAGTCGVLLLSLVITLYC

SEQ ID NO: 2 - protein, 377 aa, synthetic construct
  REGION 1..377: note = IL15-IL15Ra
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 3 - protein, 20 aa, Homo sapiens
  REGION 1..20: note = MISC_FEATURE - IL-2 derived signal peptide
MYRMQLLSCIALSLALVTNS

SEQ ID NO: 4 - protein, 21 aa, Homo sapiens
  REGION 1..21: note = MISC_FEATURE - CD8 derived signal peptide
MALPVTALLLPLALLLHAARP

SEQ ID NO: 5 - protein, 174 aa, Homo sapiens
  REGION 1..174: note = MISC_FEATURE - Intracellular domain of TNFR2
KKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 6 - protein, 37 aa, Homo sapiens
  REGION 1..37: note = MISC_FEATURE - Intracellular domain of OX40
RRDQRLPPDAHKPPGGGSFRTPIQEEQADAHSTLAKI

SEQ ID NO: 7 - protein, 60 aa, Homo sapiens
  REGION 1..60: note = MISC_FEATURE - Intracellular domain of HVEM
CVKRRKPRGDVVKVIVSVQRKRQEAEGEATVIEALQAPPDVTTVAVEETIPSFTGRSPNH

SEQ ID NO: 8 - protein, 48 aa, Homo sapiens
  REGION 1..48: note = MISC_FEATURE - Intracellular domain of CD27
QRRKYRSNKGESPVEPAEPCRYSCPREEEGSTIPIQEDYRKPEPACSP

SEQ ID NO: 9 - protein, 42 aa, Homo sapiens
  REGION 1..42: note = MISC_FEATURE - Intracellular domain of CD137
KRGRKKLLYIFKQPFMRPVQTTQEEDGCSCRFPEEEEGGCEL

SEQ ID NO: 10 - protein, 189 aa, Homo sapiens
  REGION 1..189: note = MISC_FEATURE - Intracellular domain of CD30
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 11 - protein, 193 aa, Homo sapiens
  REGION 1..193: note = MISC_FEATURE - Intracellular domain of DR3
RHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 12 - protein, 41 aa, Homo sapiens
  REGION 1..41: note = MISC_FEATURE - Intracellular domain of CD28
RSKRSRLLHSDYMNMTPRRPGPTRKHYQPYAPPRDFAAYRS

SEQ ID NO: 13 - protein, 35 aa, Homo sapiens
  REGION 1..35: note = MISC_FEATURE - Intracellular domain of ICOS
TKKKYSSSVHDPNGEYMFMRAVNTAKKSRLTDVTL

SEQ ID NO: 14 - protein, 18 aa, Thosea asigna virus
  REGION 1..18: note = self-cleaving peptide T2A of Thosea asigna virus
EGRGSLLTCGDVEENPGP

SEQ ID NO: 15 - protein, 62 aa, Homo sapiens
  REGION 1..62: note = MISC_FEATURE - CD8 derived linker peptide
SIMYFSHFVPVFLPAKPTTTPAPRPPTPAPTIASQPLSLRPEACRPAAGGAVHTRGLDFACD

SEQ ID NO: 16 - protein, 24 aa, Homo sapiens
  REGION 1..24: note = MISC_FEATURE - CD8 derived transmembrane domain
IYIWAPLAGTCGVLLLSLVITLYC

SEQ ID NO: 17 - protein, 133 aa, Homo sapiens [sequence omitted]

SEQ ID NO: 18 - protein, 129 aa, Homo sapiens [sequence omitted]

SEQ ID NO: 19 - protein, 184 aa, Homo sapiens
  REGION 1..184: note = MISC_FEATURE - IL6
PVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 20 - protein, 152 aa, Homo sapiens
  REGION 1..152: note = MISC_FEATURE - IL7
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 21 - protein, 126 aa, Homo sapiens
  REGION 1..126: note = MISC_FEATURE - IL9
QGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 22 - protein, 114 aa, Homo sapiens
  REGION 1..114: note = MISC_FEATURE - IL15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 23 - protein, 138 aa, Homo sapiens
  REGION 1..138: note = MISC_FEATURE - IL21
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 24 - protein, 112 aa, Homo sapiens
  REGION 1..112: note = MISC_FEATURE - Intracellular domain of CD3
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 294 aa, Homo sapiens
  REGION 1..294: note = MISC_FEATURE - Extracellular domain and transmembrane domain of CD19
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 26 - protein, 242 aa, synthetic construct
  REGION 1..242: note = Anti-CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 27 - protein, 15 aa, synthetic construct
  REGION 1..15: note = Flexible linker peptide
GGGGSGGGGSGGGGS

SEQ ID NO: 28 - protein, 8 aa, synthetic construct
  REGION 1..8: note = FLAG tag
DYKDDDDK

SEQ ID NO: 29 - protein, 39 aa, Homo sapiens
  REGION 1..39: note = MISC_FEATURE - CD28 derived linker peptide
IEVMYPPPYLDNEKSNGTIIHVKGKHLCPSPLFPGPSKP

SEQ ID NO: 30 - protein, 30 aa, Homo sapiens
  REGION 1..30: note = MISC_FEATURE - CD28 derived transmembrane domain
FWVLVVVGGVLACYSLLVTVAFIIFWVRSK

SEQ ID NO: 31 - protein, 30 aa, Homo sapiens
  REGION 1..30: note = MISC_FEATURE - 30 aa of CD8 derived linker peptide
ASQPLSLRPEACRPAAGGAVHTRGLDFACD

SEQ ID NO: 32 - protein, 15 aa, Homo sapiens
  REGION 1..15: note = MISC_FEATURE - 15 aa of CD8 derived linker peptide
AGGAVHTRGLDFACD

SEQ ID NO: 33 - protein, 114 aa, Mus musculus
  REGION 1..114: note = MISC_FEATURE - IL15
NWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 34 - protein, 115 aa, Rattus norvegicus
  REGION 1..115: note = MISC_FEATURE - IL15
MNWIDVRYDLEKIESLIQFIHIDTTLYTDSDFHPSCKVTAMNCFLLELQVILHEYSNMTLNETVRNVLYLANSTLSSNKNVIESGCKECEELEERNFTEFLQSFIHIVQMFINTS

SEQ ID NO: 35 - protein, 149 aa, Mus musculus
  REGION 1..149: note = MISC_FEATURE - IL2
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 36 - protein, 135 aa, Rattus norvegicus
  REGION 1..135: note = MISC_FEATURE - IL2
APTSSPAKETQQHLEQLLLDLQVLLRGIDNYKNLKLPMMLTFKFYLPKQATELKHLQCLENELGALQRVLDLTQSKSFHLEDAGNFISNIRVTVVKLKGSENKFECQFDDEPATVVEFLRRWIAICQSIISTMTQ

SEQ ID NO: 37 - protein, 129 aa, Mus musculus
  REGION 1..129: note = MISC_FEATURE - IL7
ECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 38 - protein, 129 aa, Rattus norvegicus
  REGION 1..129: note = MISC_FEATURE - IL7
DCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 39 - protein, 186 aa, Mus musculus
  REGION 1..186: note = MISC_FEATURE - Intracellular domain of TNFR2
KKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 40 - protein, 186 aa, Rattus norvegicus
  REGION 1..186: note = MISC_FEATURE - Intracellular domain of TNFR2
KKKPSCLQRETMVPHLPDDKSQDAIGLEQQHLLTTAPSSSSSSLESSASAGDRRAPPGGHPQARVTAEAQGSQEACAGSRSSDSSHGSHGTHVNVTCIVNVCSSSDHSSQCSSQASTTVGDPDANPSGSPKDEQVPFSQEECPSQSQWETTETLQNHDKPFPLGVPDVGMKPNQPGWYDQIAVKVP

SEQ ID NO: 41 - protein, 36 aa, Mus musculus
  REGION 1..36: note = MISC_FEATURE - Intracellular domain of OX40
RKAWRLPNTPKPCWGNSFRTPIQEEHTDAHFTLAKI

SEQ ID NO: 42 - protein, 36 aa, Rattus norvegicus
  REGION 1..36: note = MISC_FEATURE - Intracellular domain of OX40
RKAWRSPNTPKPCWGNSFRTPIQEEQTDTHFTLAKI

SEQ ID NO: 43 - protein, 44 aa, Mus musculus
  REGION 1..44: note = MISC_FEATURE - Intracellular domain of HVEM
TRRHLHTSSVAKELEPFQEQQENTIRFPVTEVGFAETEEETASN

SEQ ID NO: 44 - protein, 51 aa, Rattus norvegicus
  REGION 1..51: note = MISC_FEATURE - Intracellular domain of HVEM
RKQRQRHTSIVASELEAFQQEQQEDAIRFPVIEVGPSVTEEEAAFNCMNSG

SEQ ID NO: 45 - protein, 47 aa, Mus musculus
  REGION 1..47: note = MISC_FEATURE - Intracellular domain of CD27
QRRNHGPNEDRQAVPEEPCPYSCPREEEGSAIPIQEDYRKPEPAFYP

SEQ ID NO: 46 - protein, 48 aa, Rattus norvegicus
  REGION 1..48: note = MISC_FEATURE - Intracellular domain of CD27
HQRRNHGPNEDSQAVPEELCPYSCPREEEGSVIPIQEDYRKPEPASYP

SEQ ID NO: 47 - protein, 48 aa, Mus musculus
  REGION 1..48: note = MISC_FEATURE - Intracellular domain of CD137
SVLKWIRKKFPHIFKQPFKKTTGAAQEEDACSCRCPQEEEGGGGGYEL

SEQ ID NO: 48 - protein, 48 aa, Rattus norvegicus
  REGION 1..48: note = MISC_FEATURE - Intracellular domain of CD137
SVPKWLRKKFPHIFKQPFKKAVRTAQEEDACSCRFPEEEEGGGGSYEL

SEQ ID NO: 49 - DNA, 489 bp, Homo sapiens
  misc_feature 1..489: note = IL15 coding region
  source 1..489: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 50 - DNA, 1386 bp, Homo sapiens
  misc_feature 1..1386: note = TNFR2 coding region
  source 1..1386: mol_type = other DNA
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaa

SEQ ID NO: 51 - DNA, 708 bp, Homo sapiens
  misc_feature 1..708: note = CD8 coding region
  source 1..708: mol_type = other DNA
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaa

SEQ ID NO: 52 - protein, 257 aa, synthetic construct
  REGION 1..257: note = IL15TM-OX40[linker and TM is derived from CD8 ]
MYRMQLLSCIALSLALVTNSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSIMYFSHFVPVFLPAKPTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKI

SEQ ID NO: 53 - protein, 262 aa, synthetic construct
  REGION 1..262: note = IL15TM-CD137 linker and TM is derived from CD8 ]
MYRMQLLSCIALSLALVTNSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSIMYFSHFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCEL

SEQ ID NO: 54 - protein, 295 aa, synthetic construct
  REGION 1..295: note = IL7TM-OX40[linker and TM is derived from CD8 ]
MYRMQLLSCIALSLALVTNSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSIMYFSHFVPVFLPAKPTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKI

SEQ ID NO: 55 - protein, 300 aa, synthetic construct
  REGION 1..300: note = IL7TM-CD137[linker and TM is derived from CD8 ]
MYRMQLLSCIALSLALVTNSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSIMYFSHFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCEL

SEQ ID NO: 56 - protein, 104 aa, synthetic construct
  REGION 1..104: note = Neo-2/15
GSHMPKKKIQLHAEHALYDALMILNIVKTNSPPAEEKLEDYAFNFELILEEIARLFESGDQKDEAEKAKRMKEWMKRIKTTASEDEQEEMANAIITILQSWIFS